(19) United States
(12) Patent Application Publication
(10) Pub. No.: US 2024/0052297 A1
(43) Pub. Date: Feb. 15, 2024

(54) MESOPHILIC, METHYLOTROPHIC BACTERIA FOR THE PH-INDEPENDENT PRODUCTION OF BIOCHEMICALS

(71) Applicant: Acies Bio d.o.o., Ljubljana (SI)

(72) Inventors: Aleksander Johannes Kruis, Bled (SI); David Virant, Dob (SI); Mirjan Švagelj, Murska Sobota (SI); Gregor Kosec, Ljubljana (SI); Štefan Fujs, Ljubljana (SI)

(21) Appl. No.: 18/029,607

(22) PCT Filed: Sep. 30, 2021
(86) PCT No.: PCT/EP2021/076962

§ 371 Date: Apr. 2, 2023

(30) Foreign Application Priority Data
Oct. 2, 2020 (EP) 20199855.6

(51) Int. Cl. C12N 1/20; C12N 9/88; C12P 13/00; C12N 9/10

(52) U.S. Cl. CPC C12N 1/205; C12N 9/88; C12P 13/005; C12N 9/1096; C12Y 401/01015; C12Y 401/01071; C12Y 206/01019; C12R 2001/01

(57) ABSTRACT

The present invention generally relates to the biotechnology engineering, and specifically to genetically modified mesophilic, methylotrophic bacteria, which allow for the production of biochemicals in a pH-independent manner. More specifically, the present invention provides a mesophilic, methylotrophic bacterium which expresses a polypeptide which has glutamate decarboxylase (GAD) activity and is catalytically active at a pH above 5.5. The present invention further provides a mesophilic, methylotrophic bacterium which expresses a) a polypeptide having α-ketoglutarate decarboxylase (KDC) activity and b) a polypeptide having GABA transaminase (GABA-TA) activity. The present invention also provides methods for the pH-independent production of biochemicals, such as γ-amino butyric acid (GABA) or related compounds, using a bacterium of the invention.

### TECHNICAL FIELD OF THE INVENTION

The present invention generally relates to the biotechnology engineering, and specifically to mesophilic, methylotrophic bacteria, which allow for the production of biochemicals in a pH-independent manner. More specifically, the present invention provides a mesophilic, methylotrophic bacterium which expresses a polypeptide which has glutamate decarboxylase (GAD) activity and is catalytically active at a pH above 5.5. The present invention further provides a mesophilic, methylotrophic bacterium which expresses a) a polypeptide having α-ketoglutarate decarboxylase (KDC) activity and b) a polypeptide having GABA transaminase (GABA-TA) activity. The present invention also provides methods for the pH-independent production of biochemicals, such as γ-amino butyric acid (GABA) or related compounds, using a bacterium of the invention.

### BACKGROUND OF THE INVENTION

γ-amino butyric acid (GABA) is a ubiquitous non-proteinogenic amino acid, that plays various metabolic and regulatory roles in microbes, plants, animals and humans. Commercially, GABA is sold as a food supplement that promotes relaxation. GABA is also a bulk chemical that can be converted chemically or biologically to 2-pyrrolidone, which can be further converted to other bulk chemicals, such as N-methyl-2-pyrrolidone and Nylon-4. GABA and 2-pyrrolidone are currently produced from oil, which ultimately results in the release of greenhouse gasses, particularly C02, into the atmosphere and contributes to global warming. To transition to a C02-neutral society, alternative production processes for GABA and 2-pyrrolidone are required.

Sustainable GABA and 2-pyrrolidone production can be achieved by microbial conversion of carbohydrates or methanol. Carbohydrates have been the dominant substrate used for microbial chemical production due to their widespread availability. While carbohydrates may be a sustainable substrate in terms of atmospheric C02 release, they compete with food and feed chains. Because of the growing global population, carbohydrate-derived substrates will become less and less sustainable in the long term. On the other hand, biobased methanol production from wood biomass or CO2-sequestration does not interfere with food and feed production.

Bacteria that can utilize methanol for growth and product formation (methylotrophs) have been identified. Examples include Bacillus methanolicus, Methylobacterium extorquens, Methylobacillus glycogenes and Methylobacillus flagellatus. Methylotrophic bacteria can be divided into two broad groups based on the underlying metabolism that they use to assimilate methanol. In both groups, methanol is first converted to formaldehyde using a methanol dehydrogenase. The first group of methylotrophic bacteria use the Ribulose-monophosphate (RuMP) cycle to react formaldehyde with RuMP to form C6-compounds that are then metabolized further. B. methanolicus and Methylobacilli belong to the RuMP cycle group of methylotrophs. The second group, which includes M. extorquens, assimilate formaldehyde by reacting it with glycine, forming serine in the pathway known as the serine cycle. Both groups of methylotrophic bacteria have been used for the production of various biochemicals from methanol. However, chemical production with methylotrophs is not as well-developed than carbohydrate-based fermentations, including GABA or 2-pyrrolidone.

To produce GABA, most GABA-producing bacteria use the glutamate decarboxylase (GAD) enzyme, which converts glutamate to GABA through the removal of CO2. In bacteria, the GAD enzyme participates in the acid stress response. When some bacteria are exposed to low pH, they increase expression of the gad gene. This leads to rapid decarboxylation of glutamate, which consumes intracellular protons and increases the pH. GABA is produced in the process. Since GAD activity is required in acidic environments, it shows optimal activity at pH 4.0-5.0, but is inactive at neutral pH 7.0. The lack of GAD activity under normal physiological conditions of the cytosol, in particular neutral pH, prevents continuous GABA production at neutral pH. To stimulate GABA production, several studies have utilized a pH drop (FIG. 1A), where external pH is reduced to stimulate GAD activity and extracellular glutamate import.

Alternatively, mutant GAD enzymes have been developed with improved activity at neutral pH. Two approaches have been used to improve the pH range of GAD enzymes. One method removed part of the C-terminus of GAD, which physically blocks the catalytic site of GAD when the environmental pH is neutral. By truncating the C-terminus of the Lactobacillus brevis and Lactobacillus plantarum GAD enzyme, the pH range of the enzyme was expanded to 4.0-8.0 (Yu et al., 2012). In the second approach, specific sites on the GAD enzymes were mutated via random mutagenesis to obtain mutant enzyme active at neutral pH. By mutating the penultimate His residue of the E. coli GAD (H465), the C-terminus was again unable to block GAD activity at neutral pH (Pennacchieti, et al., 2009). An E89Q mutation in E. coli GAD also prevents the loss of activity at neutral pH (Thu Ho et al., 2013). A similar effect was observed in Lactobacillus brevis GAD where four amino acid changes were introduced (T171, D294G, E312S, Q346H).

Some lactic acid bacteria (LAB) are natural producers of GABA. Examples include Lactobacillus brevis and Lactobacillus buchneri, which formed 345 mM (35.5 g/L) and 251 mM (28.5 g/L) GABA at pH 5.0. However, the high titres of GABA were only observed when L-glutamate was added externally, which is economically unfeasible. Some studies have engineered organisms that produce GABA directly from glucose by expressing pH-insensitive GAD enzymes (FIG. 1B). This enabled production of 38.6 g/L GABA from glucose in a fed-batch process by Corynebacterium glutamicum, a natural glutamate producer.

Other genes related to GABA metabolism are involved in the GABA shunt, which is mostly used for controlling intracellular GABA levels and GABA degradation. In the first step, GABA is deaminated to succinate semialdehyde by GABA transaminase. In the second step, succinate semialdehyde is oxidized to succinate by succinate semialdehyde dehydrogenase, which enters the tricarboxylic acid (TCA) cycle.

GABA production directly from methanol was previously described in the thermophilic methylotroph Bacillus methanolicus MGA3. This bacterium is a natural glutamate overproducer and can form as much as 59 g/L glutamate from methanol. By expressing a heterologous thermostable GAD enzyme from Sulfobacillus thermosulfidooxidans that converts glutamate to GABA, B. methanolicus MGA3 could convert methanol to GABA (Irla et al., 2017). However, high GABA titers could only be produced in a two-phase process, where methanol was first converted to glutamate at pH 6.5. pH then had to be lowered to 4.6 to stimulate GAD activity and GABA formation (FIG. 1B). The pH-drop resulted in the production of 9 g/L GABA from methanol, which was 90-fold higher compared to a process without a pH drop (Irla et al., 2017). However, such a two-stage bioprocess is not commercially viable on large scale. The thermophilic nature of B. methanolicus MGA3 additionally complicates direct GABA formation from methanol, since most known GAD enzymes are not active at high temperatures. To enable GABA production in bioreactors, thermophilic GAD enzymes had to be used (Irla et al., 2017). However, thermophilic GAD enzymes are sensitive to pH and thus require a pH-drop and two-phase bioprocess to produce GABA from methanol with B. methanolicus MGA3. As a consequence, GABA has not been produced continuously from methanol, which is economically unfeasible.

GABA can be further converted to 2-pyrrolidone via a single enzymatic reaction. To date, only conversion of glutamate to GABA and further to 2-pyrrolidone (FIG. 1C) has been demonstrated (Zhang et al., 2016). Direct conversion of methanol to 2-pyrrolidone has not been shown. This pathway can only function when GABA is continuously produced in the absence of a pH drop. 2-pyrrolidone production from methanol therefore faces the same problems as pH-independent GABA production.

### SUMMARY OF THE INVENTION

The object of the present invention is to provide means allowing a more efficient production of biochemicals, such as GABA or related compounds. More particularly, it is an object of the present invention to provide means allowing the production of biochemicals, such as GABA or related compounds, at higher nominal yield directly from methanol.

This is achieved by the present inventors who have engineered different mesophilic, methylotrophic bacteria that are able to produce biochemicals such as GABA and 2-pyrrolidone, from methanol independently of a pH drop. This has not been reported before and will make GABA production from methanol economically feasible.

The present invention thus provides in a first aspect a mesophilic, methylotrophic bacterium which expresses a polypeptide which has glutamate decarboxylase (GAD) activity and is pH-insensitive. In other words, a bacterium of this aspect of the present invention expresses a GAD enzyme, such as a GAD mutant, which shows catalytic activity at a pH above 5.5, preferably in the range of pH 6 to pH 8, more preferably at pH 7.0.

The present invention further provides in a second aspect a mesophilic, methylotrophic bacterium which expresses a) a polypeptide having α-ketoglutarate decarboxylase (KDC) activity and b) a polypeptide having GABA transaminase (GABA-TA) activity.

The present invention further provides a further aspect a method for producing a biochemical comprising cultivating a bacterium of the present invention under suitable culture conditions in a culture medium comprising a suitable substrate, such as methanol.

The present invention may be summarized by the following items:
1. A mesophilic, methylotrophic bacterium which expresses a polypeptide which has glutamate decarboxylase (GAD) activity and is catalytically active at a pH above 5.5.
2. The bacterium according to item 1, wherein the polypeptide is catalytically active at a pH in the range of pH 6 to pH 8.
3. The bacterium according to item 1 or 2, wherein the polypeptide is catalytically active at a pH in the range of pH 6.5 to pH 7.5.
4. The bacterium according to any one of items 1 to 3, wherein the polypeptide is catalytically active at pH 7.0.
5. The bacterium according to any one of items 1 to 4, where the polypeptide is a GAD mutant comprising in its amino acid sequence at least one amino acid substitution compared to the wild type GAD enzyme from which it is derived rending the mutant active at a pH above 5.5, preferably active in the range of pH 6 to pH 8.
6. The bacterium according to item 5, wherein the amino acid substitution is in a position corresponding to position 89 and/or position 465 of SEQ ID NO: 1.
7. The bacterium according to any one of items 1 to 6, wherein the polypeptide is a GAD mutant having 2 to 20 amino acids, such as 2 to 14 amino acids, deleted from the C-terminus compared to the wild type GAD enzyme from which it is derived.
8. The bacterium according to any one of items 1 to 4, wherein the polypeptide is selected from any one of the following:
   a) a polypeptide having glutamate decarboxylase (GAD) activity which comprises an amino acid sequence having at least 70%, such as at least 80%, sequence identity with SEQ ID NO: 1 and comprising an amino acid substitution at a position corresponding to position 89 and/or position 465 in SEQ ID NO: 1;
   b) a polypeptide having glutamate decarboxylase (GAD) activity which comprises an amino acid sequence having at least 70%, such as at least 80%, sequence identity with SEQ ID NO: 2 and comprising an amino acid substitution at a position corresponding to position 84 and/or position 457 in SEQ ID NO: 2;
   c) a polypeptide having glutamate decarboxylase (GAD) activity which comprises an amino acid sequence having at least 70%, such as at least 80%, sequence identity with SEQ ID NO: 3 and comprising an amino acid substitution at a position corresponding to position 93 and/or position 467 in SEQ ID NO: 3;
   d) a polypeptide having glutamate decarboxylase (GAD) activity which comprises an amino acid sequence having at least 70%, such as at least 80%, sequence identity with SEQ ID NO: 4 and comprising an amino acid substitution at a position corresponding to position 90 and/or position 467 in SEQ ID NO: 4;
   e) a polypeptide having glutamate decarboxylase (GAD) activity which comprises an amino acid sequence having at least 70%, such as at least 80%, sequence identity with SEQ ID NO: 5 and comprising an amino acid substitution at a position corresponding to position 85 and/or position 462 in SEQ ID NO: 5;
   f) a polypeptide having glutamate decarboxylase (GAD) activity which comprises an amino acid sequence having at least 70%, such as at least 80%, sequence identity with SEQ ID NO: 6 and comprising an amino acid substitution at a position corresponding to position 89 and/or position 465 in SEQ ID NO: 6;
   g) a polypeptide having glutamate decarboxylase (GAD) activity which comprises an amino acid sequence having at least 70%, such as at least 80%, sequence identity with SEQ ID NO: 7 and comprising an amino acid substitution at a position corresponding to position 89 and/or position 466 in SEQ ID NO: 7;
   h) a polypeptide having glutamate decarboxylase (GAD) activity which comprises an amino acid sequence having at least 70%, such as at least 80%, sequence identity with SEQ ID NO: 8 and comprising an amino acid substitution at a position corresponding to position 89 and/or position 466 in SEQ ID NO: 8;
   i) a polypeptide having glutamate decarboxylase (GAD) activity which comprises an amino acid sequence having at least 70%, such as at least 80%, sequence identity with SEQ ID NO: 9 and comprising an amino acid substitution at a position corresponding to position 89 and/or position 463 in SEQ ID NO: 9;
   j) a polypeptide having glutamate decarboxylase (GAD) activity which comprises an amino acid sequence having at least 70%, such as at least 80%, sequence identity with SEQ ID NO: 10 and comprising an amino acid substitution at a position corresponding to position 17, position 91, position 94, position 213, position 346 and/or position 468 in SEQ ID NO: 10; and
   k) a polypeptide having glutamate decarboxylase (GAD) activity which comprises an amino acid sequence having at least 70%, such as at least 80%, sequence identity with SEQ ID NO: 1, 2, 3, 4, 5, 6, 7, 8, 9 or 10 wherein 2 to 20 amino acids, such as 2 to 14 amino acids, are deleted from the C-terminus.
9. The bacterium according to any one of items 1 to 4, wherein the polypeptide comprises an amino acid sequence having at least 70%, such as at least 80%, sequence identity with SEQ ID NO: 1 and comprising an amino acid substitution at a position corresponding to position 89 and/or position 465 in SEQ ID NO: 1.
10. The bacterium according to any one of items 1 to 4, wherein the polypeptide is selected from any one of the following:
   aa) a polypeptide having glutamate decarboxylase (GAD) activity which comprises the amino acid sequence of SEQ ID NO: 11, 12 or 13 or an amino acid sequence corresponding to SEQ ID NO: 11, 12 or 13, wherein 1 to 40, such as 1 to 20, amino acids are substituted with the proviso that the amino acid at position 89 is not E and/or the amino acid at position 465 is not H, preferably with the proviso that the amino acid at position 89 is Q and/or the amino acid at position 465 is A;
   bb) a polypeptide having glutamate decarboxylase (GAD) activity which comprises the amino acid sequence of SEQ ID NO: 14, 15 or 16 or an amino acid sequence corresponding to SEQ ID NO: 14, 15 or 16 wherein 1 to 40, such as 1 to 20, amino acids are substituted with the proviso that the amino acid at position 84 is not E and/or the amino acid at position 457 is not H, preferably with the proviso that the amino acid at position 84 is Q and/or the amino acid at position 457 is A;
   cc) a polypeptide having glutamate decarboxylase (GAD) activity which comprises the amino acid sequence of SEQ ID NO: 17, 18 or 19 or an amino acid sequence corresponding to SEQ ID NO: 17, 18 or 19 wherein 1 to 40, such as 1 to 20, amino acids are substituted with the proviso that the amino acid at position 93 is not E and/or the amino acid at position 467 is not H, preferably with the proviso that the amino acid at position 93 is Q and/or the amino acid at position 467 is A;
   dd) a polypeptide having glutamate decarboxylase (GAD) activity which comprises the amino acid sequence of SEQ ID NO: 20, 21 or 22 or an amino acid sequence corresponding to SEQ ID NO: 20, 21 or 22 wherein 1 to 40, such as 1 to 20, amino acids are substituted with the proviso that the amino acid at position 90 is not E and/or the amino acid at position 467 is not H, preferably with the proviso that the amino acid at position 90 is Q and/or the amino acid at position 467 is A;
   ee) a polypeptide having glutamate decarboxylase (GAD) activity which comprises the amino acid sequence of SEQ ID NO: 23, 24 or 25 or an amino acid sequence corresponding to SEQ ID NO: 23, 24 or 25 wherein 1 to 40, such as 1 to 20, amino acids are substituted with the proviso that the amino acid at position 85 is not E and/or the amino acid at position 462 is not H, preferably with the proviso that the amino acid at position 85 is Q and/or the amino acid at position 462 is A;
   ff) a polypeptide having glutamate decarboxylase (GAD) activity which comprises the amino acid sequence of SEQ ID NO: 26, 27 or 28 or an amino acid sequence corresponding to SEQ ID NO: 26, 27 or 28 wherein 1 to 40, such as 1 to 20, amino acids are substituted with the proviso that the amino acid at position 89 is not E and/or the amino acid at position 465 is not H, preferably with the proviso that the amino acid at position 89 is Q and/or the amino acid at position 465 is A;
   gg) a polypeptide having glutamate decarboxylase (GAD) activity which comprises the amino acid sequence of SEQ ID NO: 29, 30 or 31 or an amino acid sequence corresponding to SEQ ID NO: 29, 30 or 31 wherein 1 to 40, such as 1 to 20, amino acids are substituted with the proviso that the amino acid at position 89 is not E and/or the amino acid at position 466 is not H, preferably with the proviso that the amino acid at position 89 is Q and/or the amino acid at position 466 is A;
   hh) a polypeptide having glutamate decarboxylase (GAD) activity which comprises the amino acid sequence of SEQ ID NO: 32, 33 or 34 or an amino acid sequence corresponding to SEQ ID NO: 32, 33 or 34 wherein 1 to 40, such as 1 to 20, amino acids are substituted with the proviso that the amino acid at position 89 is not E and/or the amino acid at position 466 is not H, preferably with the proviso that the amino acid at position 89 is Q and/or the amino acid at position 466 is A;
   ii) a polypeptide having glutamate decarboxylase (GAD) activity which comprises the amino acid sequence of SEQ ID NO: 35, 36 or 37 or an amino acid sequence corresponding to SEQ ID NO: 35, 36 or 37 wherein 1 to 40, such as 1 to 20, amino acids are substituted with the proviso that the amino acid at position 89 is not E and/or the amino acid at position 463 is not H, preferably with the proviso that the amino acid at position 89 is Q and/or the amino acid at position 463 is A;
   jj) a polypeptide having glutamate decarboxylase (GAD) activity which comprises the amino acid sequence of SEQ ID NO: 38 or an amino acid sequence corresponding to SEQ ID NO: 38 wherein 1 to 40, such as 1 to 20, amino acids are substituted with the proviso that the amino acid at position 17 is not T, the amino acid at position 94 is not D, the amino acid at position 213 is not E and/or the amino acid at position 346 is not Q, preferably with the proviso that the amino acid at position 17 is I, the amino acid at position 94 is G, the amino acid at position 213 is S and/or the amino acid at position 346 is H; and
   kk) a polypeptide having glutamate decarboxylase (GAD) activity which comprises an amino acid sequence having at least 70%, such as at least 80%, sequence identity with any one of SEQ ID NOs: 11 to 38, wherein 2 to 20 amino acids, such as 2 to 14 amino acids, are deleted from the C-terminus.
11. The bacterium according to any one of items 1 to 4, wherein the polypeptide comprises the amino acid sequence of SEQ ID NO: 11, 12 or 13 or an amino acid sequence corresponding to SEQ ID NO: 11, 12, or 13, wherein 1 to 40, such as 1 to 20, amino acids are substituted with the proviso that the amino acid at position 89 is not E and/or the amino acid at position 465 is not H, preferably with the proviso that the amino acid at position 89 is Q and/or the amino acid at position 465 is A.
12. The bacterium according to any one of items 1 to 4, wherein the polypeptide comprises the amino acid sequence of SEQ ID NO: 90 or an amino acid sequence corresponding to SEQ ID NO: 90, wherein 1 to 40, such as 1 to 20, amino acids are substituted with the proviso that the amino acid at position 89 is not E, preferably with the proviso that the amino acid at position 89 is Q.
13. A mesophilic, methylotrophic bacterium which expresses a) a polypeptide having α-ketoglutarate decarboxylase (KDC) activity and b) a polypeptide having GABA transaminase (GABA-TA) activity.
14. The bacterium according to item 13, wherein the polypeptide having KDC activity comprises the amino acid sequence of SEQ ID NO: 39, 40, 41 or 42 or an amino acid sequence having at least 70%, such as at least 80%, sequence identity with SEQ ID NO: 39, 40, 41 or 42.
15. The bacterium according to item 13 or 14, wherein the polypeptide having KDC activity comprises the amino acid sequence of SEQ ID NO: 40 or an amino acid sequence having at least 70%, such as at least 80%, sequence identity with SEQ ID NO: 40.
16. The bacterium according to any one of items 13 to 15, wherein the polypeptide having GABA-TA activity comprises the amino acid sequence of any one of SEQ ID NOs: 43 to 74 or an amino acid sequence having at least 70%, such as at least 80%, sequence identity with any one of SEQ ID NO: 43 to 74.
17. The bacterium according to any one of items 13 to 15, wherein the polypeptide having GABA-TA activity comprises the amino acid sequence of SEQ ID NO: 43 or an amino acid sequence having at least 70%, such as at least 80%, sequence identity with SEQ ID NO: 43.
18. The bacterium according to any one of items 1 to 17, wherein said bacterium belongs to the genus Methylobacillus or Methylobacterium.
19. The bacterium according to any one of items 1 to 18, wherein said bacterium is selected from Methylobacillus flagellatus, Methylobacillus glycogenes, Methylobacillus pratensis, Methylobacillus rhizosphaerae, Methylobacterium extorquens and Methylobacterium organophilum.
20. The bacterium according to any one of items 1 to 19, wherein said bacterium is Methylobacillus flagellatus.
21. The bacterium according to any one of items 1 to 20, wherein the bacterium further expresses a polypeptide having pyrrolidone synthase (PYS) activity.
22. The bacterium according to item 21, wherein the polypeptide having pyrrolidone synthase (PYS) activity comprises the amino acid sequence of SEQ ID NO: 75 or an amino acid sequence having at least 70%, such as at least 80%, sequence identity with SEQ ID NO: 75.
23. Method for the production of a biochemical comprising cultivating a bacterium according to any one of items 1 to 22 under suitable culture conditions in a culture medium comprising a reduced one-carbon compound, such as methanol, or a multi-carbon compound that contains no carbon-carbon bonds, such as dimethyl ether and dimethylamine.
24. Method according to item 23, wherein the culture medium comprises methanol.
25. The method according to item 23 or 24, wherein the method is for producing GABA or a derivative thereof.
26. The method according to item 25, wherein the GABA derivative is selected from the group consisting of 2-pyrrolidone, N-methyl-2-pyrrolidone and polybutyrolactam.
27. The method according to item 23 or 24, wherein the method is for producing an 2-pyrrolidone or a derivative thereof, such as N-methyl-2-pyrrolidone.

### BRIEF DESCRIPTION OF THE FIGURES

FIG. 1: Existing processes for the production of GABA and 2-pyrrolidone from methanol or glucose. A—Two stage production of GABA from glucose or methanol. The substrate is first converted to glutamate and exported. After the external pH is lowered, the glutamate is reimported and converted to GABA as a part of the acid stress response. B—One stage GABA production from glucose, where a pH-independent GAD enzyme directly converts glutamate into GABA without an external pH drop. C—2-pyrrolidone formation by a pyrrolidone synthase enzyme, which converts GABA to 2-pyrrolidone.

FIG. 2: Novel and inventive approach for direct, pH-independent GABA and 2-pyrrolidone synthesis from methanol. A—comparison of the two approaches that can both be used to form GABA in a pH-independent manner. In both cases, GABA can be converted to 2-pyrrolidone. B—The novel GAD-bypass pathway which uses a α-ketoglutarate decarboxylase (KDC) and the reverse reaction of GABA-transaminase (GABA-TA) to produce GABA from methanol in a pH-independent manner.

FIG. 3: Sequence alignment of GAD enzymes.

FIG. 4: Fluorescene of GFP and mCherry expressed by methylotrophic cultures.

FIG. 5: In vitro test of three GAD enzymes in strains ABME 38, ABME 39 and ABME 40.

FIG. 6: In vivo GABA production by ABME 35 expressing Sth GAD variant in shake flasks. 30 g/L glutamate was supplemented externally at the beginning of the cultivation. GABA was measured after 24 h of growth.

FIG. 7: In vivo GABA production by ABME 5-derived strains (A) and ABME 6-derived strains (B) expressing GAD variants in shake flasks.

FIG. 8: GABA production by ABME 40 in 5 L (A) and 20 L (B) pH-controlled bioreactors. The process was oxygen limited with automatic methanol addition.

FIG. 9: GABA production from methanol in ABME 126, which was derived from ABME 6 by integrating the GAD into the epsD-G cluster. The native epsD-G promoter was used to drive gene expression.

FIG. 10: GABA production by ABME 126 in 5 L pH-controlled bioreactors. The process was carbon limited with automatic methanol addition.

FIG. 11: Comparison of the conventional GAD variant and the novel GAD-bypass pathways to convert α-ketoglutarate to GABA.

FIG. 12: GABA production from methanol in shake flasks using the GAD-bypass expressed from plasmid in strains derived from ABME 5 (A) and ABME 6 (B).

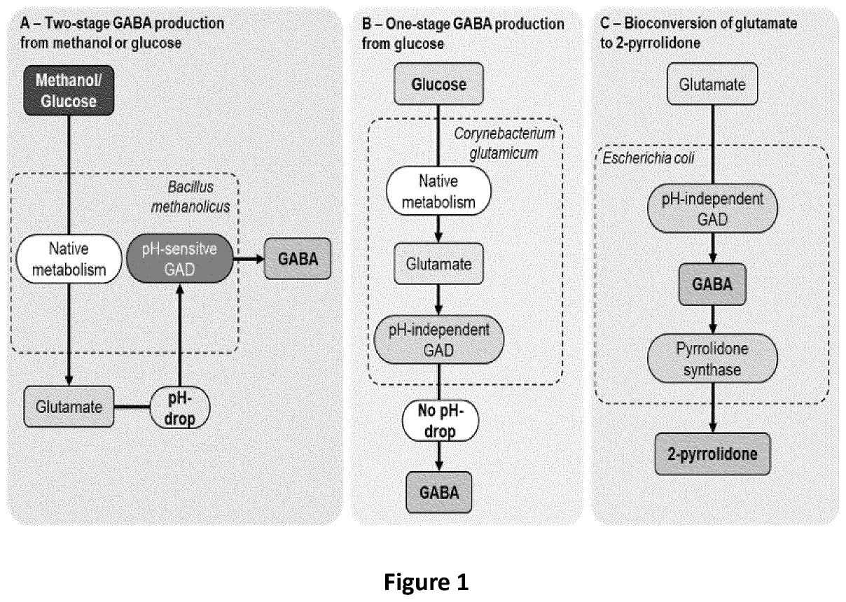

The present invention is now described in more detail below.

### DETAILED DESCRIPTION OF THE INVENTION

Unless specifically defined herein, all technical and scientific terms used have the same meaning as commonly understood by a skilled artisan in the fields of biochemistry, genetics, and microbiology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, and recombinant DNA, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory).

Bacterium of the Invention

As indicated above, the present inventors have engineered different mesophilic, methylotrophic bacteria that are able to produce biochemicals such as GABA and 2-pyrrolidone from methanol independently of a pH drop. This has not been reported before and will make the production of biochemicals from methanol economically feasible.

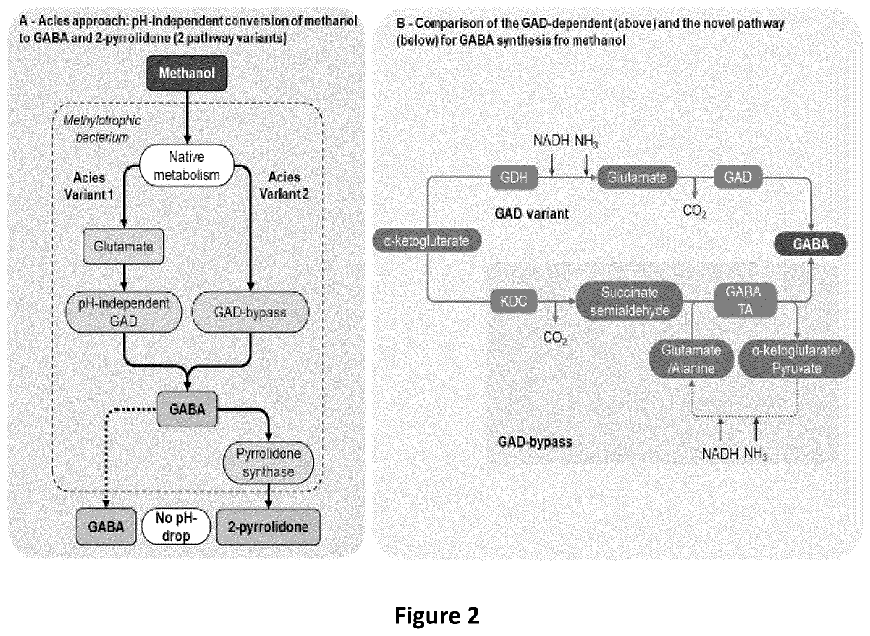

Particularly, the present inventors have developed two alternative approaches for direct, pH-independent production of biochemicals from methanol (FIG. 2). The first approach is based on mesophilic, methylotrophic bacteria which express GAD enzymes which are active under neutral to alkaline conditions, the other is based on mesophilic, methylotrophic bacteria which express two enzymes, GABA transaminase (GABA-TA) and α-ketoglutarate decarboxylase (KDC).

The present invention thus provides in a first aspect, a mesophilic, methylotrophic bacterium which expresses a polypeptide which has glutamate decarboxylase (GAD) activity and is catalytically active at a pH above 5.5.

According to some embodiments, the polypeptide having glutamate decarboxylase (GAD) activity is catalytically active at a pH in the range of pH 6 to pH 8.

According to some embodiments, the polypeptide having glutamate decarboxylase (GAD) activity is catalytically active at a pH in the range of pH 6.5 to pH 7.5.

According to some embodiments, the polypeptide having glutamate decarboxylase (GAD) activity is catalytically active at pH 7.0.

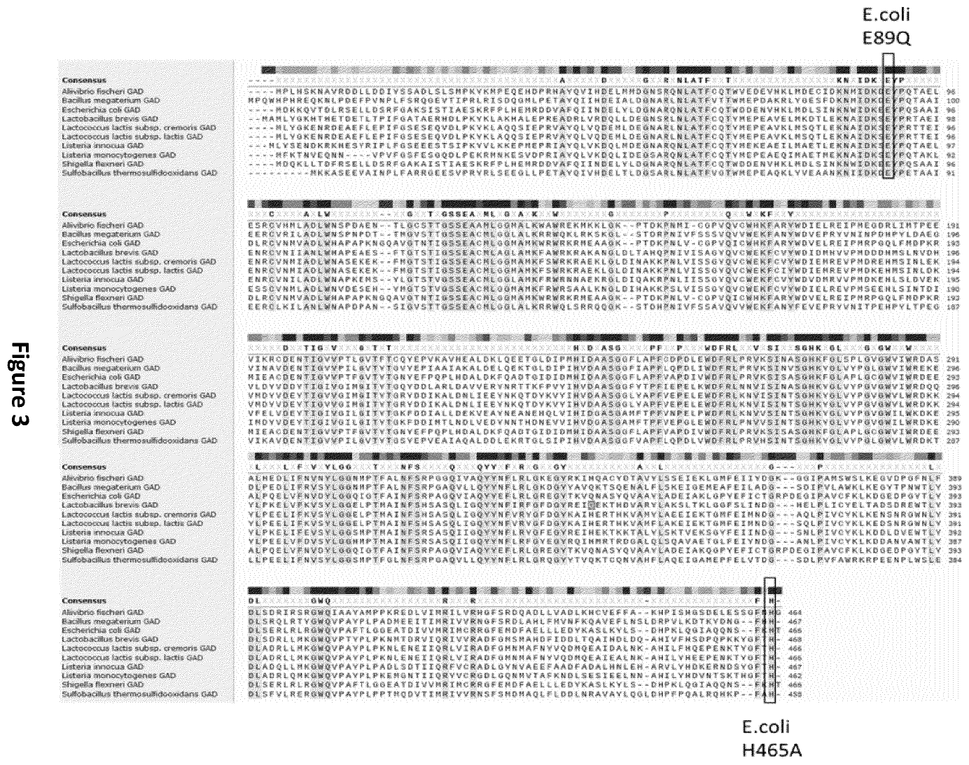

A polypeptide which has glutamate decarboxylase (GAD) activity and is catalytically active at a pH above 5.5 may be naturally occurring or may be genetically modified to improve the pH range. In the latter case, two approaches have so far been used to improve the pH range of GAD enzymes. One approach removes part of the C-terminus of GAD, which physically blocks the catalytic site of GAD when the environmental pH is neutral. In the second approach, specific sites in the GAD enzymes are mutated via random mutagenesis to obtain mutant enzyme active at neutral pH. In this respect, GAD enzymes, and particularly bacterial GAD enzymes, share several conserved regions, which containing a number of residues which have been shown to cause a shift in the pH range. By way of non-limiting example, mutating the penultimate His residue of the E. coli GAD (H465), the C-terminus was again unable to block GAD activity at neutral pH (Pennacchieti, et al., 2009). A E89Q mutation in E. coli GAD also prevents the loss of activity at neutral pH (Thu Ho et al., 2013). A similar effect was observed in Lactobacillus brevis GAD where four amino acid changes were introduced (T171, D294G, E312S, Q346H). Of particular note, the two sites that improve the pH range in E. coli are also conserved in other GAD genes (as shown in FIG. 3).

Thus, according to some embodiments, the polypeptide having glutamate decarboxylase (GAD) activity and being active at a pH above 5.5 is a GAD mutant, such as mutant derived from a bacterial GAD enzyme.

According to some embodiments, the polypeptide having glutamate decarboxylase (GAD) activity is a GAD mutant comprising in its amino acid sequence at least one amino acid substitution compared to the wild type GAD enzyme from which it is derived rending the mutant active at a pH above 5.5.

According to some embodiments, the polypeptide having glutamate decarboxylase (GAD) activity is a GAD mutant comprising in its amino acid sequence at least one amino acid substitution compared to the wild type GAD enzyme from which it is derived rending the mutant active at a pH in the range of pH 6 to pH 8.

According to some embodiments, the polypeptide having glutamate decarboxylase (GAD) activity is a GAD mutant comprising in its amino acid sequence at least one amino acid substitution compared to the wild type GAD enzyme from which it is derived rending the mutant active at a pH in the range of pH 6.5 to pH 7.5.

According to some embodiments, the polypeptide having glutamate decarboxylase (GAD) activity is a GAD mutant comprising in its amino acid sequence at least one amino acid substitution compared to the wild type GAD enzyme from which it is derived rending the mutant active at pH 7.0.

According to some embodiments, the at least one amino acid substitution is in a position corresponding to position 89 and/or position 465 of SEQ ID NO: 1. Thus, according to some embodiments, the GAD mutant comprises in its amino acid sequence an amino acid substitution in a position corresponding to position 89 of SEQ ID NO: 1. According to some embodiments, the GAD mutant comprises in its amino acid sequence an amino acid substitution in a position corresponding to position 465 of SEQ ID NO: 1. According to some embodiments, the GAD mutant comprises in its amino acid sequence two amino acid substitutions in positions corresponding to position 89 and position 465 of SEQ ID NO: 1.

According to some embodiments, the polypeptide is a GAD mutant having 2 to 20 amino acids, such as 2 to 14 amino acids, deleted from the C-terminus compared to the wild type GAD enzyme from which it is derived.

According to some embodiments, the polypeptide having glutamate decarboxylase (GAD) activity is selected from any one of the following:
a) a polypeptide having glutamate decarboxylase (GAD) activity which comprises an amino acid sequence having at least 70%, such as at least 80%, sequence identity with SEQ ID NO: 1 and comprising an amino acid substitution at a position corresponding to position 89 and/or position 465 in SEQ ID NO: 1;
b) a polypeptide having glutamate decarboxylase (GAD) activity which comprises an amino acid sequence having at least 70%, such as at least 80%, sequence identity with SEQ ID NO: 2 and comprising an amino acid substitution at a position corresponding to position 84 and/or position 457 in SEQ ID NO: 2;
c) a polypeptide having glutamate decarboxylase (GAD) activity which comprises an amino acid sequence having at least 70%, such as at least 80%, sequence identity with SEQ ID NO: 3 and comprising an amino acid substitution at a position corresponding to position 93 and/or position 467 in SEQ ID NO: 3;
d) a polypeptide having glutamate decarboxylase (GAD) activity which comprises an amino acid sequence having at least 70%, such as at least 80%, sequence identity with SEQ ID NO: 4 and comprising an amino acid substitution at a position corresponding to position 90 and/or position 467 in SEQ ID NO: 4;
e) a polypeptide having glutamate decarboxylase (GAD) activity which comprises an amino acid sequence having at least 70%, such as at least 80%, sequence identity with SEQ ID NO: 5 and comprising an amino acid substitution at a position corresponding to position 85 and/or position 462 in SEQ ID NO: 5;
f) a polypeptide having glutamate decarboxylase (GAD) activity which comprises an amino acid sequence having at least 70%, such as at least 80%, sequence identity with SEQ ID NO: 6 and comprising an amino acid substitution at a position corresponding to position 89 and/or position 465 in SEQ ID NO: 6;
g) a polypeptide having glutamate decarboxylase (GAD) activity which comprises an amino acid sequence having at least 70%, such as at least 80%, sequence identity with SEQ ID NO: 7 and comprising an amino acid substitution at a position corresponding to position 89 and/or position 466 in SEQ ID NO: 7;
h) a polypeptide having glutamate decarboxylase (GAD) activity which comprises an amino acid sequence having at least 70%, such as at least 80%, sequence identity with SEQ ID NO: 8 and comprising an amino acid substitution at a position corresponding to position 89 and/or position 466 in SEQ ID NO: 8;
i) a polypeptide having glutamate decarboxylase (GAD) activity which comprises an amino acid sequence having at least 70%, such as at least 80%, sequence identity with SEQ ID NO: 9 and comprising an amino acid substitution at a position corresponding to position 89 and/or position 463 in SEQ ID NO: 9;
j) a polypeptide having glutamate decarboxylase (GAD) activity which comprises an amino acid sequence having at least 70%, such as at least 80%, sequence identity with SEQ ID NO: 10 and comprising an amino acid substitution at a position corresponding to position 17, position 91, position 94, position 213, position 346 and/or position 468 in SEQ ID NO: 10; and
k) a polypeptide having glutamate decarboxylase (GAD) activity which comprises an amino acid sequence having at least 70%, such as at least 80%, sequence identity with SEQ ID NO: 1, 2, 3, 4, 5, 6, 7, 8, 9 or 10 wherein 2 to 20 amino acids, such as 2 to 14 amino acids, are deleted from the C-terminus.

According to some embodiments, the polypeptide having glutamate decarboxylase (GAD) activity comprises an amino acid sequence which has at least 70%, such as at least 75%, sequence identity with SEQ ID NO: 1 and comprises an amino acid substitution at a position corresponding to position 89 and/or position 465 in SEQ ID NO: 1. According to some embodiments, the polypeptide having glutamate decarboxylase (GAD) activity comprises an amino acid sequence which has at least 80%, such as at least 85%, sequence identity with SEQ ID NO: 1 and comprises an amino acid substitution at a position corresponding to position 89 and/or position 465 in SEQ ID NO: 1. According to some embodiments, the polypeptide having glutamate decarboxylase (GAD) activity comprises an amino acid sequence which has at least 90%, such as at least 95%, sequence identity with SEQ ID NO: 1 and comprises an amino acid substitution at a position corresponding to position 89 and/or position 465 in SEQ ID NO: 1.

According to some embodiments, the polypeptide having glutamate decarboxylase (GAD) activity comprises an amino acid sequence which has at least 70%, such as at least 75%, sequence identity with SEQ ID NO: 1 and comprises an amino acid substitution at a position corresponding to position 89 in SEQ ID NO: 1. According to some embodiments, the polypeptide having glutamate decarboxylase (GAD) activity comprises an amino acid sequence which has at least 80%, such as at least 85%, sequence identity with SEQ ID NO: 1 and comprises an amino acid substitution at a position corresponding to position 89 in SEQ ID NO: 1. According to some embodiments, the polypeptide having glutamate decarboxylase (GAD) activity comprises an amino acid sequence which has at least 90%, such as at least 95%, sequence identity with SEQ ID NO: 1 and comprises an amino acid substitution at a position corresponding to position 89 in SEQ ID NO: 1.

According to some embodiments, the polypeptide having glutamate decarboxylase (GAD) activity comprises an amino acid sequence which has at least 70%, such as at least 75%, sequence identity with SEQ ID NO: 1 and comprises an amino acid substitution at a position corresponding to position 465 in SEQ ID NO: 1. According to some embodiments, the polypeptide having glutamate decarboxylase (GAD) activity comprises an amino acid sequence which has at least 80%, such as at least 85%, sequence identity with SEQ ID NO: 1 and comprises an amino acid substitution at a position corresponding to position 465 in SEQ ID NO: 1. According to some embodiments, the polypeptide having glutamate decarboxylase (GAD) activity comprises an amino acid sequence which has at least 90%, such as at least 90%, sequence identity with SEQ ID NO: 1 and comprises an amino acid substitution at a position corresponding to position 465 in SEQ ID NO: 1.

According to some embodiments, the polypeptide having glutamate decarboxylase (GAD) activity comprises an amino acid sequence which has at least 70%, such as at least 75%, sequence identity with SEQ ID NO: 1 and comprises an amino acid substitution at a position corresponding to position 89 and position 465 in SEQ ID NO: 1. According to some embodiments, the polypeptide having glutamate decarboxylase (GAD) activity comprises an amino acid sequence which has at least 80%, such as at least 85%, sequence identity with SEQ ID NO: 1 and comprises an amino acid substitution at a position corresponding to position 89 and position 465 in SEQ ID NO: 1. According to some embodiments, the polypeptide having glutamate decarboxylase (GAD) activity comprises an amino acid sequence which has at least 90%, such as at least 95%, sequence identity with SEQ ID NO: 1 and comprises an amino acid substitution at a position corresponding to position 89 and position 465 in SEQ ID NO: 1.

According to some embodiments, the polypeptide having glutamate decarboxylase (GAD) activity comprises an amino acid sequence which has at least 70%, such as at least 75%, sequence identity with SEQ ID NO: 2 and comprises an amino acid substitution at a position corresponding to position 84 and/or position 457 in SEQ ID NO: 2. According to some embodiments, the polypeptide having glutamate decarboxylase (GAD) activity comprises an amino acid sequence which has at least 80%, such as at least 85%, sequence identity with SEQ ID NO: 2 and comprises an amino acid substitution at a position corresponding to position 84 and/or position 457 in SEQ ID NO: 2. According to some embodiments, the polypeptide having glutamate decarboxylase (GAD) activity comprises an amino acid sequence which has at least 90%, such as at least 95%, sequence identity with SEQ ID NO: 2 and comprises an amino acid substitution at a position corresponding to position 84 and/or position 457 in SEQ ID NO: 2.

According to some embodiments, the polypeptide having glutamate decarboxylase (GAD) activity comprises an amino acid sequence which has at least 70%, such as at least 75%, sequence identity with SEQ ID NO: 2 and comprises an amino acid substitution at a position corresponding to position 84 in SEQ ID NO: 2. According to some embodiments, the polypeptide having glutamate decarboxylase (GAD) activity comprises an amino acid sequence which has at least 80%, such as at least 85%, sequence identity with SEQ ID NO: 2 and comprises an amino acid substitution at a position corresponding to position 84 in SEQ ID NO: 2. According to some embodiments, the polypeptide having glutamate decarboxylase (GAD) activity comprises an amino acid sequence which has at least 90%, such as at least 95%, sequence identity with SEQ ID NO: 2 and comprises an amino acid substitution at a position corresponding to position 84 in SEQ ID NO: 2.

According to some embodiments, the polypeptide having glutamate decarboxylase (GAD) activity comprises an amino acid sequence which has at least 70%, such as at least 75%, sequence identity with SEQ ID NO: 2 and comprises an amino acid substitution at a position corresponding to position 457 in SEQ ID NO: 2. According to some embodiments, the polypeptide having glutamate decarboxylase (GAD) activity comprises an amino acid sequence which has at least 80%, such as at least 85%, sequence identity with SEQ ID NO: 2 and comprises an amino acid substitution at a position corresponding to position 457 in SEQ ID NO: 2. According to some embodiments, the polypeptide having glutamate decarboxylase (GAD) activity comprises an amino acid sequence which has at least 90%, such as at least 95%, sequence identity with SEQ ID NO: 2 and comprises an amino acid substitution at a position corresponding to position 457 in SEQ ID NO: 2.

According to some embodiments, the polypeptide having glutamate decarboxylase (GAD) activity comprises an amino acid sequence which has at least 70%, such as at least 75%, sequence identity with SEQ ID NO: 2 and comprises an amino acid substitution at a position corresponding to position 84 and position 457 in SEQ ID NO: 2. According to some embodiments, the polypeptide having glutamate decarboxylase (GAD) activity comprises an amino acid sequence which has at least 80%, such as at least 85%, sequence identity with SEQ ID NO: 2 and comprises an amino acid substitution at a position corresponding to position 84 and position 457 in SEQ ID NO: 2. According to some embodiments, the polypeptide having glutamate decarboxylase (GAD) activity comprises an amino acid sequence which has at least 90%, such as at least 95%, sequence identity with SEQ ID NO: 2 and comprises an amino acid substitution at a position corresponding to position 84 and position 457 in SEQ ID NO: 2.

According to some embodiments, the polypeptide having glutamate decarboxylase (GAD) activity comprises an amino acid sequence which has at least 70%, such as at least 75%, sequence identity with SEQ ID NO: 3 and comprises an amino acid substitution at a position corresponding to position 93 and/or position 467 in SEQ ID NO: 3. According to some embodiments, the polypeptide having glutamate decarboxylase (GAD) activity comprises an amino acid sequence which has at least 80%, such as at least 85%, sequence identity with SEQ ID NO: 3 and comprises an amino acid substitution at a position corresponding to position 93 and/or position 467 in SEQ ID NO: 3. According to some embodiments, the polypeptide having glutamate decarboxylase (GAD) activity comprises an amino acid sequence which has at least 90%, such as at least 95%, sequence identity with SEQ ID NO: 3 and comprises an amino acid substitution at a position corresponding to position 93 and/or position 467 in SEQ ID NO: 3.

According to some embodiments, the polypeptide having glutamate decarboxylase (GAD) activity comprises an amino acid sequence which has at least 70%, such as at least 75%, sequence identity with SEQ ID NO: 4 and comprises an amino acid substitution at a position corresponding to position 90 and/or position 467 in SEQ ID NO: 4. According to some embodiments, the polypeptide having glutamate decarboxylase (GAD) activity comprises an amino acid sequence which has at least 80%, such as at least 85%, sequence identity with SEQ ID NO: 4 and comprises an amino acid substitution at a position corresponding to position 90 and/or position 467 in SEQ ID NO: 4. According to some embodiments, the polypeptide having glutamate decarboxylase (GAD) activity comprises an amino acid sequence which has at least 90%, such as at least 95%, sequence identity with SEQ ID NO: 4 and comprises an amino acid substitution at a position corresponding to position 90 and/or position 467 in SEQ ID NO: 4.

According to some embodiments, the polypeptide having glutamate decarboxylase (GAD) activity comprises an amino acid sequence which has at least 70%, such as at least 75%, sequence identity with SEQ ID NO: 5 and comprises an amino acid substitution at a position corresponding to position 85 and/or position 462 in SEQ ID NO: 5. According to some embodiments, the polypeptide having glutamate decarboxylase (GAD) activity comprises an amino acid sequence which has at least 80%, such as at least 85%, sequence identity with SEQ ID NO: 5 and comprises an amino acid substitution at a position corresponding to position 85 and/or position 462 in SEQ ID NO: 5. According to some embodiments, the polypeptide having glutamate decarboxylase (GAD) activity comprises an amino acid sequence which has at least 90%, such as at least 95%, sequence identity with SEQ ID NO: 5 and comprises an amino acid substitution at a position corresponding to position 85 and/or position 462 in SEQ ID NO: 5.

According to some embodiments, the polypeptide having glutamate decarboxylase (GAD) activity comprises an amino acid sequence which has at least 70%, such as at least 75%, sequence identity with SEQ ID NO: 6 and comprises an amino acid substitution at a position corresponding to position 89 and/or position 465 in SEQ ID NO: 6. According to some embodiments, the polypeptide having glutamate decarboxylase (GAD) activity comprises an amino acid sequence which has at least 80%, such as at least 85%, sequence identity with SEQ ID NO: 6 and comprises an amino acid substitution at a position corresponding to position 89 and/or position 465 in SEQ ID NO: 6. According to some embodiments, the polypeptide having glutamate decarboxylase (GAD) activity comprises an amino acid sequence which has at least 90%, such as at least 95%, sequence identity with SEQ ID NO: 6 and comprises an amino acid substitution at a position corresponding to position 89 and/or position 465 in SEQ ID NO: 6.

According to some embodiments, the polypeptide having glutamate decarboxylase (GAD) activity comprises an amino acid sequence which has at least 70%, such as at least 75%, sequence identity with SEQ ID NO: 7 and comprises an amino acid substitution at a position corresponding to position 89 and/or position 466 in SEQ ID NO: 7. According to some embodiments, the polypeptide having glutamate decarboxylase (GAD) activity comprises an amino acid sequence which has at least 80%, such as at least 85%, sequence identity with SEQ ID NO: 7 and comprises an amino acid substitution at a position corresponding to position 89 and/or position 466 in SEQ ID NO: 7. According to some embodiments, the polypeptide having glutamate decarboxylase (GAD) activity comprises an amino acid sequence which has at least 90%, such as at least 95%, sequence identity with SEQ ID NO: 7 and comprises an amino acid substitution at a position corresponding to position 89 and/or position 466 in SEQ ID NO: 7.

According to some embodiments, the polypeptide having glutamate decarboxylase (GAD) activity comprises an amino acid sequence which has at least 70%, such as at least 75%, sequence identity with SEQ ID NO: 8 and comprises an amino acid substitution at a position corresponding to position 89 and/or position 466 in SEQ ID NO: 8. According to some embodiments, the polypeptide having glutamate decarboxylase (GAD) activity comprises an amino acid sequence which has at least 80%, such as at least 85%, sequence identity with SEQ ID NO: 8 and comprises an amino acid substitution at a position corresponding to position 89 and/or position 466 in SEQ ID NO: 8. According to some embodiments, the polypeptide having glutamate decarboxylase (GAD) activity comprises an amino acid sequence which has at least 90%, such as at least 95%, sequence identity with SEQ ID NO: 8 and comprises an amino acid substitution at a position corresponding to position 89 and/or position 466 in SEQ ID NO: 8.

According to some embodiments, the polypeptide having glutamate decarboxylase (GAD) activity comprises an amino acid sequence which has at least 70%, such as at least 75%, sequence identity with SEQ ID NO: 9 and comprises an amino acid substitution at a position corresponding to position 89 and/or position 463 in SEQ ID NO: 9. According to some embodiments, the polypeptide having glutamate decarboxylase (GAD) activity comprises an amino acid sequence which has at least 80%, such as at least 85%, sequence identity with SEQ ID NO: 9 and comprises an amino acid substitution at a position corresponding to position 89 and/or position 463 in SEQ ID NO: 9. According to some embodiments, the polypeptide having glutamate decarboxylase (GAD) activity comprises an amino acid sequence which has at least 90%, such as at least 95%, sequence identity with SEQ ID NO: 9 and comprises an amino acid substitution at a position corresponding to position 89 and/or position 463 in SEQ ID NO: 9.

According to some embodiments, the polypeptide having glutamate decarboxylase (GAD) activity comprises an amino acid sequence which has at least 70%, such as at least 75%, sequence identity with SEQ ID NO: 10 and comprises an amino acid substitution at a position corresponding to position 17, position 91, position 94, position 213, position 346 and/or position 468 in SEQ ID NO: 10. According to some embodiments, the polypeptide having glutamate decarboxylase (GAD) activity comprises an amino acid sequence which has at least 80%, such as at least 85%, sequence identity with SEQ ID NO: 10 and comprises an amino acid substitution at a position corresponding to position 17, position 91, position 94, position 213, position 346 and/or position 468 in SEQ ID NO: 10. According to some embodiments, the polypeptide having glutamate decarboxylase (GAD) activity comprises an amino acid sequence which has at least 90%, such as at least 95%, sequence identity with SEQ ID NO: 10 and comprises an amino acid substitution at a position corresponding to position 17, position 91, position 94, position 213, position 346 and/or position 468 in SEQ ID NO: 10.

According to some embodiments, the polypeptide having glutamate decarboxylase (GAD) activity comprises an amino acid sequence which has at least 70%, such as at least 75%, sequence identity with SEQ ID NO: 10 and comprises an amino acid substitution at a position corresponding to position 17, position 94, position 213 and/or position 346 in SEQ ID NO: 10. According to some embodiments, the polypeptide having glutamate decarboxylase (GAD) activity comprises an amino acid sequence which has at least 80%, such as at least 85%, sequence identity with SEQ ID NO: 10 and comprises an amino acid substitution at a position corresponding to position 17, position 94, position 213 and/or position 346 in SEQ ID NO: 10. According to some embodiments, the polypeptide having glutamate decarboxylase (GAD) activity comprises an amino acid sequence which has at least 90%, such as at least 95%, sequence identity with SEQ ID NO: 10 and comprises an amino acid substitution at a position corresponding to position 17, position 91, position 94, position 213, position 346 and/or position 468 in SEQ ID NO: 10.

According to some embodiments, the polypeptide having glutamate decarboxylase (GAD) activity comprises an amino acid sequence having at least 70%, such as at least 75%, sequence identity with SEQ ID NO: 1, 2, 3, 4, 5, 6, 7, 8, 9 or 10 wherein 2 to 20 amino acids are deleted from the C-terminus. According to some embodiments, the polypeptide having glutamate decarboxylase (GAD) activity comprises an amino acid sequence having at least 80%, such as at least 85%, sequence identity with SEQ ID NO: 1, 2, 3, 4, 5, 6, 7, 8, 9 or 10 wherein 2 to 20 amino acids are deleted from the C-terminus. According to some embodiments, the polypeptide having glutamate decarboxylase (GAD) activity comprises an amino acid sequence having at least 90%, such as at least 95%, sequence identity with SEQ ID NO: 1, 2, 3, 4, 5, 6, 7, 8, 9 or 10 wherein 2 to 20 amino acids, are deleted from the C-terminus.

According to some embodiments, the polypeptide having glutamate decarboxylase (GAD) activity comprises an amino acid sequence having at least 70%, such as at least 75%, sequence identity with SEQ ID NO: 1, 2, 3, 4, 5, 6, 7, 8, 9 or 10 wherein 2 to 14 amino acids are deleted from the C-terminus. According to some embodiments, the polypeptide having glutamate decarboxylase (GAD) activity comprises an amino acid sequence having at least 80%, such as at least 85%, sequence identity with SEQ ID NO: 1, 2, 3, 4, 5, 6, 7, 8, 9 or 10 wherein 2 to 14 amino acids are deleted from the C-terminus. According to some embodiments, the polypeptide having glutamate decarboxylase (GAD) activity comprises an amino acid sequence having at least 90%, such as at least 95%, sequence identity with SEQ ID NO: 1, 2, 3, 4, 5, 6, 7, 8, 9 or 10 wherein 2 to 14 amino acids are deleted from the C-terminus.

According to some embodiments, the polypeptide having glutamate decarboxylase (GAD) activity comprises an amino acid sequence having at least 70%, such as at least 75%, sequence identity with SEQ ID NO: 1, 2, 3, 4, 5, 6, 7, 8, 9 or 10 wherein 4 to 10 amino acids are deleted from the C-terminus. According to some embodiments, the polypeptide having glutamate decarboxylase (GAD) activity comprises an amino acid sequence having at least 80%, such as at least 85%, sequence identity with SEQ ID NO: 1, 2, 3, 4, 5, 6, 7, 8, 9 or 10 wherein 4 to 10 amino acids are deleted from the C-terminus. According to some embodiments, the polypeptide having glutamate decarboxylase (GAD) activity comprises an amino acid sequence having at least 90%, such as at least 95%, sequence identity with SEQ ID NO: 1, 2, 3, 4, 5, 6, 7, 8, 9 or 10 wherein 4 to 10 amino acids are deleted from the C-terminus.

According to some embodiments, the polypeptide having glutamate decarboxylase (GAD) activity is selected from any one of the following:
aa) a polypeptide having glutamate decarboxylase (GAD) activity which comprises the amino acid sequence of SEQ ID NO: 11, 12 or 13 or an amino acid sequence corresponding to SEQ ID NO: 11, 12 or 13, wherein 1 to 40, such as 1 to 20, amino acids are substituted with the proviso that the amino acid at position 89 is not E and/or the amino acid at position 465 is not H, preferably with the proviso that the amino acid at position 89 is Q and/or the amino acid at position 465 is A;
bb) a polypeptide having glutamate decarboxylase (GAD) activity which comprises the amino acid sequence of SEQ ID NO: 14, 15 or 16 or an amino acid sequence corresponding to SEQ ID NO: 14, 15 or 16 wherein 1 to 40, such as 1 to 20, amino acids are substituted with the proviso that the amino acid at position 84 is not E and/or the amino acid at position 457 is not H, preferably with the proviso that the amino acid at position 84 is Q and/or the amino acid at position 457 is A;
cc) a polypeptide having glutamate decarboxylase (GAD) activity which comprises the amino acid sequence of SEQ ID NO: 17, 18 or 19 or an amino acid sequence corresponding to SEQ ID NO: 17, 18 or 19 wherein 1 to 40, such as 1 to 20, amino acids are substituted with the proviso that the amino acid at position 93 is not E and/or the amino acid at position 467 is not H, preferably with the proviso that the amino acid at position 93 is Q and/or the amino acid at position 467 is A;
dd) a polypeptide having glutamate decarboxylase (GAD) activity which comprises the amino acid sequence of SEQ ID NO: 20, 21 or 22 or an amino acid sequence corresponding to SEQ ID NO: 20, 21 or 22 wherein 1 to 40, such as 1 to 20, amino acids are substituted with the proviso that the amino acid at position 90 is not E and/or the amino acid at position 467 is not H, preferably with the proviso that the amino acid at position 90 is Q and/or the amino acid at position 467 is A;
ee) a polypeptide having glutamate decarboxylase (GAD) activity which comprises the amino acid sequence of SEQ ID NO: 23, 24 or 25 or an amino acid sequence corresponding to SEQ ID NO: 23, 24 or 25 wherein 1 to 40, such as 1 to 20, amino acids are substituted with the proviso that the amino acid at position 85 is not E and/or the amino acid at position 462 is not H, preferably with the proviso that the amino acid at position 85 is Q and/or the amino acid at position 462 is A;
ff) a polypeptide having glutamate decarboxylase (GAD) activity which comprises the amino acid sequence of SEQ ID NO: 26, 27 or 28 or an amino acid sequence corresponding to SEQ ID NO: 26, 27 or 28 wherein 1 to 40, such as 1 to 20, amino acids are substituted with the proviso that the amino acid at position 89 is not E and/or the amino acid at position 465 is not H, preferably with the proviso that the amino acid at position 89 is Q and/or the amino acid at position 465 is A;
gg) a polypeptide having glutamate decarboxylase (GAD) activity which comprises the amino acid sequence of SEQ ID NO: 29, 30 or 31 or an amino acid sequence corresponding to SEQ ID NO: 29, 30 or 31 wherein 1 to 40, such as 1 to 20, amino acids are substituted with the proviso that the amino acid at position 89 is not E and/or the amino acid at position 466 is not H, preferably with the proviso that the amino acid at position 89 is Q and/or the amino acid at position 466 is A;
hh) a polypeptide having glutamate decarboxylase (GAD) activity which comprises the amino acid sequence of SEQ ID NO: 32, 33 or 34 or an amino acid sequence corresponding to SEQ ID NO: 32, 33 or 34 wherein 1 to 40, such as 1 to 20, amino acids are substituted with the proviso that the amino acid at position 89 is not E and/or the amino acid at position 466 is not H, preferably with the proviso that the amino acid at position 89 is Q and/or the amino acid at position 466 is A;
ii) a polypeptide having glutamate decarboxylase (GAD) activity which comprises the amino acid sequence of SEQ ID NO: 35, 36 or 37 or an amino acid sequence corresponding to SEQ ID NO: 35, 36 or 37 wherein 1 to 40, such as 1 to 20, amino acids are substituted with the proviso that the amino acid at position 89 is not E and/or the amino acid at position 463 is not H, preferably with the proviso that the amino acid at position 89 is Q and/or the amino acid at position 463 is A;
jj) a polypeptide having glutamate decarboxylase (GAD) activity which comprises the amino acid sequence of SEQ ID NO: 38 or an amino acid sequence corresponding to SEQ ID NO: 38 wherein 1 to 40, such as 1 to 20, amino acids are substituted with the proviso that the amino acid at position 17 is not T, the amino acid at position 94 is not D, the amino acid at position 213 is not E and/or the amino acid at position 346 is not Q, preferably with the proviso that the amino acid at position 17 is I, the amino acid at position 94 is G, the amino acid at position 213 is S and/or the amino acid at position 346 is H; and
kk) a polypeptide having glutamate decarboxylase (GAD) activity which comprises an amino acid sequence having at least 70%, such as at least 80%, sequence identity with any one of SEQ ID NOs: 11 to 38, wherein 2 to 20 amino acids, such as 2 to 14 amino acids, are deleted from the C-terminus.

According to some embodiments, the polypeptide having glutamate decarboxylase (GAD) activity comprises the amino acid sequence of SEQ ID NO: 11, 12 or 13 or an amino acid sequence corresponding to SEQ ID NO: 11, 12 or 13, wherein 1 to 40 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 11, 12 or 13 or an amino acid sequence corresponding to SEQ ID NO: 11, 12 or 13, wherein 1 to 20 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 11, 12 or 13 or an amino acid sequence corresponding to SEQ ID NO: 11, 12 or 13, wherein 1 to 10 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 11, 12 or 13 or an amino acid sequence corresponding to SEQ ID NO: 11, 12 or 13, wherein 1 to 5 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 11, 12 or 13.

According to some embodiments, the polypeptide having glutamate decarboxylase (GAD) activity comprises the amino acid sequence of SEQ ID NO: 14, 15 or 16 or an amino acid sequence corresponding to SEQ ID NO: 14, 15 or 16 wherein 1 to 40 amino acids are substituted with the proviso that the amino acid at position 84 is not E and/or the amino acid at position 457 is not H, preferably with the proviso that the amino acid at position 84 is Q and/or the amino acid at position 457 is A. According to some embodiments, the polypeptide having glutamate decarboxylase (GAD) activity comprises the amino acid sequence of SEQ ID NO: 14, 15 or 16 or an amino acid sequence corresponding to SEQ ID NO: 14, 15 or 16 wherein 1 to 20 amino acids are substituted with the proviso that the amino acid at position 84 is not E and/or the amino acid at position 457 is not H, preferably with the proviso that the amino acid at position 84 is Q and/or the amino acid at position 457 is A. According to some embodiments, the polypeptide having glutamate decarboxylase (GAD) activity comprises the amino acid sequence of SEQ ID NO: 14, 15 or 16 or an amino acid sequence corresponding to SEQ ID NO: 14, 15 or 16 wherein 1 to 20 amino acids are substituted with the proviso that the amino acid at position 84 is not E and/or the amino acid at position 457 is not H, preferably with the proviso that the amino acid at position 84 is Q and/or the amino acid at position 457 is A. According to some embodiments, the polypeptide having glutamate decarboxylase (GAD) activity comprises the amino acid sequence of SEQ ID NO: 14, 15 or 16 or an amino acid sequence corresponding to SEQ ID NO: 14, 15 or 16 wherein 1 to 10 amino acids are substituted with the proviso that the amino acid at position 84 is not E and/or the amino acid at position 457 is not H, preferably with the proviso that the amino acid at position 84 is Q and/or the amino acid at position 457 is A. According to some embodiments, the polypeptide having glutamate decarboxylase (GAD) activity comprises the amino acid sequence of SEQ ID NO: 14, 15 or 16 or an amino acid sequence corresponding to SEQ ID NO: 14, 15 or 16 wherein 1 to 5 amino acids are substituted with the proviso that the amino acid at position 84 is not E and/or the amino acid at position 457 is not H, preferably with the proviso that the amino acid at position 84 is Q and/or the amino acid at position 457 is A. According to some embodiments, the polypeptide having glutamate decarboxylase (GAD) activity comprises the amino acid sequence of SEQ ID NO: 14, 15 or 16.

According to some embodiments, the polypeptide having glutamate decarboxylase (GAD) activity comprises the amino acid sequence of SEQ ID NO: 17, 18 or 19 or an amino acid sequence corresponding to SEQ ID NO: 17, 18 or 19 wherein 1 to 40 amino acids are substituted with the proviso that the amino acid at position 93 is not E and/or the amino acid at position 467 is not H, preferably with the proviso that the amino acid at position 93 is Q and/or the amino acid at position 467 is A. According to some embodiments, the polypeptide having glutamate decarboxylase (GAD) activity comprises the amino acid sequence of SEQ ID NO: 17, 18 or 19 or an amino acid sequence corresponding to SEQ ID NO: 17, 18 or 19 wherein 1 to 20 amino acids are substituted with the proviso that the amino acid at position 93 is not E and/or the amino acid at position 467 is not H, preferably with the proviso that the amino acid at position 93 is Q and/or the amino acid at position 467 is A. According to some embodiments, the polypeptide having glutamate decarboxylase (GAD) activity comprises the amino acid sequence of SEQ ID NO: 17, 18 or 19 or an amino acid sequence corresponding to SEQ ID NO: 17, 18 or 19 wherein 1 to 10 amino acids are substituted with the proviso that the amino acid at position 93 is not E and/or the amino acid at position 467 is not H, preferably with the proviso that the amino acid at position 93 is Q and/or the amino acid at position 467 is A. According to some embodiments, the polypeptide having glutamate decarboxylase (GAD) activity comprises the amino acid sequence of SEQ ID NO: 17, 18 or 19 or an amino acid sequence corresponding to SEQ ID NO: 17, 18 or 19 wherein 1 to 5 amino acids are substituted with the proviso that the amino acid at position 93 is not E and/or the amino acid at position 467 is not H, preferably with the proviso that the amino acid at position 93 is Q and/or the amino acid at position 467 is A. According to some embodiments, the polypeptide having glutamate decarboxylase (GAD) activity comprises the amino acid sequence of SEQ ID NO: 17, 18 or 19.

According to some embodiments, the polypeptide having glutamate decarboxylase (GAD) activity comprises the amino acid sequence of SEQ ID NO: 20, 21 or 22 or an amino acid sequence corresponding to SEQ ID NO: 20, 21 or 22 wherein 1 to 40 amino acids are substituted with the proviso that the amino acid at position 90 is not E and/or the amino acid at position 467 is not H, preferably with the proviso that the amino acid at position 90 is Q and/or the amino acid at position 467 is A. According to some embodiments, the polypeptide having glutamate decarboxylase (GAD) activity comprises the amino acid sequence of SEQ ID NO: 20, 21 or 22 or an amino acid sequence corresponding to SEQ ID NO: 20, 21 or 22 wherein 1 to 20 amino acids are substituted with the proviso that the amino acid at position 90 is not E and/or the amino acid at position 467 is not H, preferably with the proviso that the amino acid at position 90 is Q and/or the amino acid at position 467 is A. According to some embodiments, the polypeptide having glutamate decarboxylase (GAD) activity comprises the amino acid sequence of SEQ ID NO: 20, 21 or 22 or an amino acid sequence corresponding to SEQ ID NO: 20, 21 or 22 wherein 1 to 10 amino acids are substituted with the proviso that the amino acid at position 90 is not E and/or the amino acid at position 467 is not H, preferably with the proviso that the amino acid at position 90 is Q and/or the amino acid at position 467 is A. According to some embodiments, the polypeptide having glutamate decarboxylase (GAD) activity comprises the amino acid sequence of SEQ ID NO: 20, 21 or 22 or an amino acid sequence corresponding to SEQ ID NO: 20, 21 or 22 wherein 1 to 5 amino acids are substituted with the proviso that the amino acid at position 90 is not E and/or the amino acid at position 467 is not H, preferably with the proviso that the amino acid at position 90 is Q and/or the amino acid at position 467 is A. According to some embodiments, the polypeptide having glutamate decarboxylase (GAD) activity comprises the amino acid sequence of SEQ ID NO: 20, 21 or 22

According to some embodiments, the polypeptide having glutamate decarboxylase (GAD) activity comprises the amino acid sequence of SEQ ID NO: 23, 24 or 25 or an amino acid sequence corresponding to SEQ ID NO: 23, 24 or 25 wherein 1 to 40 amino acids are substituted with the proviso that the amino acid at position 85 is not E and/or the amino acid at position 462 is not H, preferably with the proviso that the amino acid at position 85 is Q and/or the amino acid at position 462 is A. According to some embodiments, the polypeptide having glutamate decarboxylase (GAD) activity comprises the amino acid sequence of SEQ ID NO: 23, 24 or 25 or an amino acid sequence corresponding to SEQ ID NO: 23, 24 or 25 wherein 1 to 20 amino acids are substituted with the proviso that the amino acid at position 85 is not E and/or the amino acid at position 462 is not H, preferably with the proviso that the amino acid at position 85 is Q and/or the amino acid at position 462 is A. According to some embodiments, the polypeptide having glutamate decarboxylase (GAD) activity comprises the amino acid sequence of SEQ ID NO: 23, 24 or 25 or an amino acid sequence corresponding to SEQ ID NO: 23, 24 or 25 wherein 1 to 10 amino acids are substituted with the proviso that the amino acid at position 85 is not E and/or the amino acid at position 462 is not H, preferably with the proviso that the amino acid at position 85 is Q and/or the amino acid at position 462 is A. According to some embodiments, the polypeptide having glutamate decarboxylase (GAD) activity comprises the amino acid sequence of SEQ ID NO: 23, 24 or 25 or an amino acid sequence corresponding to SEQ ID NO: 23, 24 or 25 wherein 1 to 5 amino acids are substituted with the proviso that the amino acid at position 85 is not E and/or the amino acid at position 462 is not H, preferably with the proviso that the amino acid at position 85 is Q and/or the amino acid at position 462 is A. According to some embodiments, the polypeptide having glutamate decarboxylase (GAD) activity comprises the amino acid sequence of SEQ ID NO: 23, 24 or 25.

According to some embodiments, the polypeptide having glutamate decarboxylase (GAD) activity comprises the amino acid sequence of SEQ ID NO: 26, 27 or 28 or an amino acid sequence corresponding to SEQ ID NO: 26, 27 or 28 wherein 1 to 40 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 26, 27 or 28 or an amino acid sequence corresponding to SEQ ID NO: 26, 27 or 28 wherein 1 to 20 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 26, 27 or 28 or an amino acid sequence corresponding to SEQ ID NO: 26, 27 or 28 wherein 1 to 10 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 26, 27 or 28 or an amino acid sequence corresponding to SEQ ID NO: 26, 27 or 28 wherein 1 to 5 amino acids are substituted with the proviso that the amino acid at position 89 is not E and/or the amino acid at position 465 is not H, preferably with the proviso that the amino acid at position 89 is Q and/or the amino acid at position 465 is A. According to some embodiments, the polypeptide having glutamate decarboxylase (GAD) activity comprises the amino acid sequence of SEQ ID NO: 26, 27 or 28.

According to some embodiments, the polypeptide having glutamate decarboxylase (GAD) activity comprises the amino acid sequence of SEQ ID NO: 29, 30 or 31 or an amino acid sequence corresponding to SEQ ID NO: 29, 30 or 31 wherein 1 to 40 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 29, 30 or 31 or an amino acid sequence corresponding to SEQ ID NO: 29, 30 or 31 wherein 1 to 20 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 29, 30 or 31 or an amino acid sequence corresponding to SEQ ID NO: 29, 30 or 31 wherein 1 to 10 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 29, 30 or 31 or an amino acid sequence corresponding to SEQ ID NO: 29, 30 or 31 wherein 1 to 5 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 29, 30 or 31.

According to some embodiments, the polypeptide having glutamate decarboxylase (GAD) activity comprises the amino acid sequence of SEQ ID NO: 32, 33 or 34 or an amino acid sequence corresponding to SEQ ID NO: 32, 33 or 34 wherein 1 to 40 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 32, 33 or 34 or an amino acid sequence corresponding to SEQ ID NO: 32, 33 or 34 wherein 1 to 20 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 32, 33 or 34 or an amino acid sequence corresponding to SEQ ID NO: 32, 33 or 34 wherein 1 to 10 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 32, 33 or 34 or an amino acid sequence corresponding to SEQ ID NO: 32, 33 or 34 wherein 1 to 5 amino acids are substituted with the proviso that the amino acid at position 89 is not E and/or the amino acid at position 466 is not H, preferably with the proviso that the amino acid at position 89 is Q and/or the amino acid at position 466 is A. According to some embodiments, the polypeptide having glutamate decarboxylase (GAD) activity comprises the amino acid sequence of SEQ ID NO: 32, 33 or 34.

According to some embodiments, the polypeptide having glutamate decarboxylase (GAD) activity comprises the amino acid sequence of SEQ ID NO: 35, 36 or 37 or an amino acid sequence corresponding to SEQ ID NO: 35, 36 or 37 wherein 1 to 40 amino acids are substituted with the proviso that the amino acid at position 89 is not E and/or the amino acid at position 463 is not H, preferably with the proviso that the amino acid at position 89 is Q and/or the amino acid at position 463 is A. According to some embodiments, the polypeptide having glutamate decarboxylase (GAD) activity comprises the amino acid sequence of SEQ ID NO: 35, 36 or 37 or an amino acid sequence corresponding to SEQ ID NO: 35, 36 or 37 wherein 1 to 20 amino acids are substituted with the proviso that the amino acid at position 89 is not E and/or the amino acid at position 463 is not H, preferably with the proviso that the amino acid at position 89 is Q and/or the amino acid at position 463 is A. According to some embodiments, the polypeptide having glutamate decarboxylase (GAD) activity comprises the amino acid sequence of SEQ ID NO: 35, 36 or 37 or an amino acid sequence corresponding to SEQ ID NO: 35, 36 or 37 wherein 1 to 10 amino acids are substituted with the proviso that the amino acid at position 89 is not E and/or the amino acid at position 463 is not H, preferably with the proviso that the amino acid at position 89 is Q and/or the amino acid at position 463 is A. According to some embodiments, the polypeptide having glutamate decarboxylase (GAD) activity comprises the amino acid sequence of SEQ ID NO: 35, 36 or 37 or an amino acid sequence corresponding to SEQ ID NO: 35, 36 or 37 wherein 1 to 5 amino acids are substituted with the proviso that the amino acid at position 89 is not E and/or the amino acid at position 463 is not H, preferably with the proviso that the amino acid at position 89 is Q and/or the amino acid at position 463 is A. According to some embodiments, the polypeptide having glutamate decarboxylase (GAD) activity comprises the amino acid sequence of SEQ ID NO: 35, 36 or 37.

According to some embodiments, the polypeptide having glutamate decarboxylase (GAD) activity comprises the amino acid sequence of SEQ ID NO: 38 or an amino acid sequence corresponding to SEQ ID NO: 38 wherein 1 to 40 amino acids are substituted with the proviso that the amino acid at position 17 is not T, the amino acid at position 94 is not D, the amino acid at position 213 is not E and/or the amino acid at position 346 is not Q, preferably with the proviso that the amino acid at position 17 is I, the amino acid at position 94 is G, the amino acid at position 213 is S and/or the amino acid at position 346 is H. According to some embodiments, the polypeptide having glutamate decarboxylase (GAD) activity comprises the amino acid sequence of SEQ ID NO: 38 or an amino acid sequence corresponding to SEQ ID NO: 38 wherein 1 to 20 amino acids are substituted with the proviso that the amino acid at position 17 is not T, the amino acid at position 94 is not D, the amino acid at position 213 is not E and/or the amino acid at position 346 is not Q, preferably with the proviso that the amino acid at position 17 is I, the amino acid at position 94 is G, the amino acid at position 213 is S and/or the amino acid at position 346 is H. According to some embodiments, the polypeptide having glutamate decarboxylase (GAD) activity comprises the amino acid sequence of SEQ ID NO: 38 or an amino acid sequence corresponding to SEQ ID NO: 38 wherein 1 to 10 amino acids are substituted with the proviso that the amino acid at position 17 is not T, the amino acid at position 94 is not D, the amino acid at position 213 is not E and/or the amino acid at position 346 is not Q, preferably with the proviso that the amino acid at position 17 is I, the amino acid at position 94 is G, the amino acid at position 213 is S and/or the amino acid at position 346 is H. According to some embodiments, the polypeptide having glutamate decarboxylase (GAD) activity comprises the amino acid sequence of SEQ ID NO: 38 or an amino acid sequence corresponding to SEQ ID NO: 38 wherein 1 to 5 amino acids are substituted with the proviso that the amino acid at position 17 is not T, the amino acid at position 94 is not D, the amino acid at position 213 is not E and/or the amino acid at position 346 is not Q, preferably with the proviso that the amino acid at position 17 is I, the amino acid at position 94 is G, the amino acid at position 213 is S and/or the amino acid at position 346 is H. According to some embodiments, the polypeptide having glutamate decarboxylase (GAD) activity comprises the amino acid sequence of SEQ ID NO: 38 or an amino acid sequence corresponding to SEQ ID NO: 38.

According to some embodiments, the polypeptide having glutamate decarboxylase (GAD) activity comprises an amino acid sequence having at least 70%, such as at least 75%, sequence identity with any one of SEQ ID NOs: 11 to 38, wherein 2 to 20 amino acids are deleted from the C-terminus. According to some embodiments, the polypeptide having glutamate decarboxylase (GAD) activity comprises an amino acid sequence having at least 80%, such as at least 85%, sequence identity with any one of SEQ ID NOs: 11 to 38, wherein 2 to 20 amino acids are deleted from the C-terminus.

According to some embodiments, the polypeptide having glutamate decarboxylase (GAD) activity comprises an amino acid sequence having at least 90%, such as at least 95%, sequence identity with any one of SEQ ID NOs: 11 to 38, wherein 2 to 20 amino acids are deleted from the C-terminus.

According to some embodiments, the polypeptide having glutamate decarboxylase (GAD) activity comprises an amino acid sequence having at least 70%, such as at least 75%, sequence identity with any one of SEQ ID NOs: 11 to 38, wherein 2 to 14 amino acids are deleted from the C-terminus. According to some embodiments, the polypeptide having glutamate decarboxylase (GAD) activity comprises an amino acid sequence having at least 80%, such as at least 85%, sequence identity with any one of SEQ ID NOs: 11 to 38, wherein 2 to 14 amino acids are deleted from the C-terminus. According to some embodiments, the polypeptide having glutamate decarboxylase (GAD) activity comprises an amino acid sequence having at least 90%, such as at least 95%, sequence identity with any one of SEQ ID NOs: 11 to 38, wherein 2 to 14 amino acids are deleted from the C-terminus.

According to some embodiments, the polypeptide having glutamate decarboxylase (GAD) activity comprises an amino acid sequence having at least 70%, such as at least 75%, sequence identity with any one of SEQ ID NOs: 11 to 38, wherein 4 to 10 amino acids are deleted from the C-terminus. According to some embodiments, the polypeptide having glutamate decarboxylase (GAD) activity comprises an amino acid sequence having at least 80%, such as at least 85%, sequence identity with any one of SEQ ID NOs: 11 to 38, wherein 4 to 10 amino acids are deleted from the C-terminus. According to some embodiments, the polypeptide having glutamate decarboxylase (GAD) activity comprises an amino acid sequence having at least 90%, such as at least 95%, sequence identity with any one of SEQ ID NOs: 11 to 38, wherein 4 to 10 amino acids are deleted from the C-terminus.

According to some embodiments, the polypeptide having glutamate decarboxylase (GAD) activity comprises the amino acid sequence of SEQ ID NO: 90 or an amino acid sequence corresponding to SEQ ID NO: 90, wherein 1 to 40, such as 1 to 20, amino acids are substituted with the proviso that the amino acid at position 89 is not E, preferably with the proviso that the amino acid at position 89 is Q.

According to some embodiments, the polypeptide having glutamate decarboxylase (GAD) activity comprises the amino acid sequence of SEQ ID NO: 92 or an amino acid sequence corresponding to SEQ ID NO: 92, wherein 1 to 40, such as 1 to 20, amino acids are substituted with the proviso that the amino acid at position 84 is not E, preferably with the proviso that the amino acid at position 84 is Q.

The present invention further provides in a second aspect a mesophilic, methylotrophic bacterium which expresses a) a polypeptide having α-ketoglutarate decarboxylase (KDC) activity and b) a polypeptide having GABA transaminase (GABA-TA) activity.

According to some embodiments, the polypeptide having KDC activity comprises the amino acid sequence of SEQ ID NO: 39, 40, 41 or 42 or an amino acid sequence having at least 70%, such as at least 80%, sequence identity with SEQ ID NO: 39, 40, 41 or 42. According to some embodiments, the polypeptide having KDC activity comprises the amino acid sequence of SEQ ID NO: 39, 40, 41 or 42. According to some embodiments, the polypeptide having KDC activity comprises an amino acid sequence having at least 70%, such as at least 80%, sequence identity with SEQ ID NO: 39, 40, 41 or 42.

According to some embodiments, the polypeptide having KDC activity comprises the amino acid sequence of SEQ ID NO: 39 or an amino acid sequence having at least 70%, such as at least 75%, sequence identity with SEQ ID NO: 39. According to some embodiments, the polypeptide having KDC activity comprises the amino acid sequence of SEQ ID NO: 39 or an amino acid sequence having at least 80%, such as at least 85%, sequence identity with SEQ ID NO: 39. According to some embodiments, the polypeptide having KDC activity comprises the amino acid sequence of SEQ ID NO: 39 or an amino acid sequence having at least 90%, such as at least 95%, sequence identity with SEQ ID NO: 39. According to some embodiments, the polypeptide having KDC activity comprises the amino acid sequence of SEQ ID NO: 39.

According to some embodiments, the polypeptide having KDC activity comprises the amino acid sequence of SEQ ID NO: 40 or an amino acid sequence having at least 70%, such as at least 75%, sequence identity with SEQ ID NO: 40. According to some embodiments, the polypeptide having KDC activity comprises the amino acid sequence of SEQ ID NO: 40 or an amino acid sequence having at least 80%, such as at least 85%, sequence identity with SEQ ID NO: 40. According to some embodiments, the polypeptide having KDC activity comprises the amino acid sequence of SEQ ID NO: 40 or an amino acid sequence having at least 90%, such as at least 95%, sequence identity with SEQ ID NO: 40. According to some embodiments, the polypeptide having KDC activity comprises the amino acid sequence of SEQ ID NO: 40.

According to some embodiments, the polypeptide having KDC activity comprises the amino acid sequence of SEQ ID NO: 41 or an amino acid sequence having at least 70%, such as at least 75%, sequence identity with SEQ ID NO: 41. According to some embodiments, the polypeptide having KDC activity comprises the amino acid sequence of SEQ ID NO: 41 or an amino acid sequence having at least 80%, such as at least 85%, sequence identity with SEQ ID NO: 41. According to some embodiments, the polypeptide having KDC activity comprises the amino acid sequence of SEQ ID NO: 41 or an amino acid sequence having at least 90%, such as at least 95%, sequence identity with SEQ ID NO: 41. According to some embodiments, the polypeptide having KDC activity comprises the amino acid sequence of SEQ ID NO: 41.

According to some embodiments, the polypeptide having KDC activity comprises the amino acid sequence of SEQ ID NO: 42 or an amino acid sequence having at least 70%, such as at least 75%, sequence identity with SEQ ID NO: 42. According to some embodiments, the polypeptide having KDC activity comprises the amino acid sequence of SEQ ID NO: 42 or an amino acid sequence having at least 80%, such as at least 85%, sequence identity with SEQ ID NO: 42. According to some embodiments, the polypeptide having KDC activity comprises the amino acid sequence of SEQ ID NO: 42 or an amino acid sequence having at least 90%, such as at least 95%, sequence identity with SEQ ID NO: 42. According to some embodiments, the polypeptide having KDC activity comprises the amino acid sequence of SEQ ID NO: 42.

According to some embodiments, the polypeptide having GABA-TA activity comprises the amino acid sequence of any one of SEQ ID NOs: 43 to 74 or an amino acid sequence having at least 70%, such as at least 75%, sequence identity with any one of SEQ ID NO: 43 to 74. According to some embodiments, the polypeptide having GABA-TA activity comprises the amino acid sequence of any one of SEQ ID NOs: 43 to 74 or an amino acid sequence having at least 80%, such as at least 85%, sequence identity with any one of SEQ ID NO: 43 to 74. According to some embodiments, the polypeptide having GABA-TA activity comprises the amino acid sequence of any one of SEQ ID NOs: 43 to 74 or an amino acid sequence having at least 90%, such as at least 95%, sequence identity with any one of SEQ ID NO: 43 to 74. According to some embodiments, the polypeptide having GABA-TA activity comprises the amino acid sequence of any one of SEQ ID NOs: 43 to 74.

According to some embodiments, the polypeptide having GABA-TA activity comprises the amino acid sequence of SEQ ID NO: 43 or an amino acid sequence having at least 70%, such as at least 75%, sequence identity with SEQ ID NO: 43. According to some embodiments, the polypeptide having GABA-TA activity comprises the amino acid sequence of SEQ ID NO: 43 or an amino acid sequence having at least 80%, such as at least 85%, sequence identity with SEQ ID NO: 43. According to some embodiments, the polypeptide having GABA-TA activity comprises the amino acid sequence of SEQ ID NO: 43 or an amino acid sequence having at least 90%, such as at least 95%, sequence identity with SEQ ID NO: 43. According to some embodiments, the polypeptide having GABA-TA activity comprises the amino acid sequence of SEQ ID NO: 43.

The present invention also contemplates the conversion of GABA into 2-pyrrolidone via a single enzymatic reaction. Thus, according to some embodiments, a bacterium of the present invention further expresses a polypeptide having pyrrolidone synthase (PYS) activity.

According to some embodiments, the polypeptide having pyrrolidone synthase (PYS) activity comprises the amino acid sequence of SEQ ID NO: 75 or an amino acid sequence having at least 70%, such as at least 75%, sequence identity with SEQ ID NO: 75. According to some embodiments, the polypeptide having pyrrolidone synthase (PYS) activity comprises the amino acid sequence of SEQ ID NO: 75 or an amino acid sequence having at least 80%, such as at least 85%, sequence identity with SEQ ID NO: 75. According to some embodiments, the polypeptide having pyrrolidone synthase (PYS) activity comprises the amino acid sequence of SEQ ID NO: 75 or an amino acid sequence having at least 90%, such as at least 95%, sequence identity with SEQ ID NO: 75.

Generally, a polypeptide as detailed herein and employed in accordance with the invention will be heterologous to the bacterium of the invention, which means that said polypeptide is normally not found in or made (i.e. expressed) by the bacterium of the invention, but derived from a different species. However, the present invention also contemplates embodiments, where only one of the polypeptide having α-ketoglutarate decarboxylase (KDC) activity and the polypeptide having GABA transaminase (GABA-TA) activity is heterologous to the bacterium, e.g. where the polypeptide having α-ketoglutarate decarboxylase (KDC) activity is heterologous, while the polypeptide having GABA transaminase (GABA-TA) activity is endogenous to the bacterium.

A bacterium of the invention may thus have been modified to express at least one polypeptide as detailed herein, which means that an exogenous nucleic acid molecule, such as a DNA molecule, which comprises a nucleotide sequence encoding said polypeptide has been introduced in the bacterium. Thus, a bacterium of the present invention may comprise an exogenous nucleic acid molecule, such as a DNA molecule, which comprises a nucleotide sequence encoding the polypeptide in question. Techniques for introducing an exogenous nucleic acid molecule, such as a DNA molecule, into a bacterial cell are well-known to those of skill in the art, and include transformation (e.g., heat shock or natural transformation) among others.

In order to facilitate (over)expression of a polypeptide in the bacterium, the exogenous nucleic acid molecule may comprise suitable regulatory elements such as a promoter that is functional in the bacterial cell to cause the production of an mRNA molecule and that is operably linked to the nucleotide sequence encoding said polypeptide.

Promoters useful in accordance with the invention are any known promoters that are functional in a given host cell to cause the production of an mRNA molecule. Many such promoters are known to the skilled person. Such promoters include promoters normally associated with other genes, and/or promoters isolated from any bacteria. The use of promoters for protein expression is generally known to those of skilled in the art of molecular biology, for example, see Sambrook et al., Molecular cloning: A Laboratory Manual, Cold Spring Harbor Laboratory, Cold Spring Harbor, N.Y., 1989. The promoter employed may be inducible, such as a temperature inducible promoter (e.g., a pL or pR phage lambda promoters, each of which can be controlled by the temperature-sensitive lambda repressor c1857). The term “inducible” used in the context of a promoter means that the promoter only directs transcription of an operably linked nucleotide sequence if a stimulus is present, such as a change in temperature or the presence of a chemical substance (“chemical inducer”). As used herein, “chemical induction” according to the present invention refers to the physical application of an exogenous or endogenous substance (incl. macromolecules, e.g., proteins or nucleic acids) to a host cell. This has the effect of causing the target promoter present in the host cell to increase the rate of transcription. Alternatively, the promoter employed may be constitutive. The term “constitutive” used in the context of a promoter means that the promoter is capable of directing transcription of an operably linked nucleotide sequence in the absence of stimulus (such as heat shock, chemicals etc.).

Temperature induction systems work, for example, by employing promoters that are repressed by thermolabile repressors. These repressors are active at lower temperatures for example at 30° C., while unable to fold correctly at 37° C. and are therefore inactive. Such circuits therefore can be used to directly regulate the genes of interest (St-Pierre et al. 2013) also by genome integration of the genes along with the repressors. Examples of such as a temperature inducible expression system are based on the pL and/or pR λ phage promoters which are regulated by the thermolabile c1857 repressor. Similar to the genome integrated DE3 system, the expression of the T7 RNA polymerase gene may also be controlled using a temperature controlled promoter system (Mertens et al. 1995), while the expression of the genes of interest can be controlled using a T7 promoter.

Non-limiting examples of promoters functional in bacteria include both constitutive and inducible promoters such as T7 promoter, the beta-lactamase and lactose promoter systems; alkaline phosphatase (phoA) promoter, a tryptophan (trp) promoter system, tetracycline promoter, lambda-phage promoter, ribosomal protein promoters; and hybrid promoters such as the tac promoter. Other bacterial and synthetic promoters are also suitable.

Besides a promoter, the exogenous nucleic acid molecule may further comprise at least one regulatory element selected from a 5′ untranslated region (5′UTR) and 3′ untranslated region (3′ UTR). Many such 5′ UTRs and 3′ UTRs derived from prokaryotes and eukaryotes are well known to the skilled person. Such regulatory elements include 5′ UTRs and 3′ UTRs normally associated with other genes, and/or 5′ UTRs and 3′ UTRs isolated from any bacteria.

Usually, the 5′ UTR contains a ribosome binding site (RBS), also known as the Shine Dalgarno sequence which is usually 3-10 base pairs upstream from the initiation codon.

The exogenous nucleic acid molecule may be a vector or part of a vector, such as an expression vector. Normally, such a vector remains extrachromosomal within the bacterial cell which means that it is found outside of the nucleus or nucleoid region of the bacterium.

It is also contemplated by the present invention that the exogenous nucleic acid molecule is stably integrated into the genome of the bacterium. Means for stable integration into the genome of a host cell, e.g., by homologous recombination, are well known to the skilled person.

A bacterium in accordance with the present invention can be produced from any suitable mesophilic and methylotrophic bacterium. The bacterium may be Gram-positive or Gram-negative.

According to some embodiments, the bacterium of the present invention is of the genus Methylobacillus or Methylobacterium.

According to some embodiments, the bacterium of the present invention is selected from Methylobacillus flagellatus, Methylobacillus glycogenes, Methylobacillus pratensis, Methylobacillus rhizosphaerae, Methylobacterium extorquens and Methylobacterium organophilum.

According to some embodiments, the bacterium of the present invention is Methylobacillus flagellutes.

According to some embodiments, the bacterium of the present invention is Methylobacillus glycogenes.

According to some embodiments, the bacterium of the present invention is Methylobacillus pratensis.

According to some embodiments, the bacterium of the present invention is Methylobacillus rhizosphaerae.

According to some embodiments, the bacterium of the present invention is Methylobacterium extorquens.

According to some embodiments, the bacterium of the present invention is Methylobacterium organophilum.

Method of the Invention

The present invention also provides methods for producing a biochemical comprising cultivating a bacterium according to the invention under suitable culture conditions in a culture medium comprising a reduced one-carbon compound, such as methanol, or a multi-carbon compound that contain no carbon-carbon bonds, such as dimethyl ether and dimethylamine.

The method may further comprise collecting the biochemical from the culture medium.

According to certain embodiments, present invention provides a method for producing GABA or a derivative thereof. Particularly, the present invention provides a method for producing GABA or a derivative thereof; said method comprises cultivating a bacterium as detailed herein in a culture medium. The method may further comprise collecting GABA or the derivative thereof from the culture medium.

According to some embodiments, the GABA derivative is selected from the group consisting of 2-pyrrolidone, N-methyl-2-pyrrolidone and polybutyrolactam.

According to certain embodiments, present invention provides a method for producing 2-pyrrolidone or a derivative thereof, such as N-methyl-2-pyrrolidone. Particularly, the present invention provides a method for producing 2-pyrrolidone or a derivative thereof, such as N-methyl-pyrrolidone, said method comprises cultivating a bacterium as detailed herein in a culture medium. The method may further comprise collecting 2-pyrrolidone or the derivative thereof, such as N-methyl-2-pyrrolidone, from the culture medium.

The culture medium employed may be any conventional medium suitable for culturing a bacterium cell in question, and may be composed according to the principles of the prior art. The medium will usually contain all nutrients necessary for the growth and survival of the respective bacterium, such as carbon and nitrogen sources and other inorganic salts. Suitable media, e.g. minimal or complex media, are available from commercial suppliers, or may be prepared according to published receipts, e.g. the American Type Culture Collection (ATCC) Catalogue of strains. Non-limiting standard medium well known to the skilled person include Luria Bertani (LB) broth, Sabouraud Dextrose (SD) broth, MS broth, Yeast Peptone Dextrose, BMMY, GMMY, or Yeast Malt Extract (YM) broth, which are all commercially available. A non-limiting example of suitable media for culturing bacterial cells, such as E. coli cells, including minimal media and rich media such as Luria Broth (LB), M9 media, M17 media, SA media, MOPS media, Terrific Broth, YT and others.

The carbon source may be any suitable carbon substrate known in the art, and in particularly any carbon substrate commonly used in the cultivation of methylotrophic bacteria and/or fermentation. A carbon source of particular interest is a reduced one-carbon compound, such as methanol, methane, formate, or methylamine, or a multi-carbon compound that contains no carbon-carbon bonds, such as dimethyl ether and dimethylamine. Thus, according to some embodiments, the culture medium comprises methanol as a carbon source. The concentration of methanol in the culture medium may generally be between in the range from about 0.5% w/v to about 4% w/v, such as from about 2% w/v to about 4% w/v. According to some embodiments, the concentration of methanol in the culture medium is in the range from about 2.5% w/v to about 3.5% w/v.

As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used.

The cultivation can be preferably performed under aerobic conditions, such as by a shaking culture, and by a stirring culture with aeration, at a temperature of about 20 to about 45° C., such as about 30 to 38° C., such as at about 37° C. The pH of the culture is usually above 5, such as in a range from about 6 to about 8, preferably from about 6.5 to about 7.5, more preferably from about 6.8 to about 7.2. The pH of the culture can be adjusted with ammonia, calcium carbonate, various acids, various bases, and buffers. The cultivation may be carried out for a period in the range from 10 to 70 h, preferably in a range from 24 to 60 h, more preferably in a range from 36 to 50 h.

After cultivation, solids such as cells can be removed from the culture medium by centrifugation or membrane filtration. The biochemical can be collected by conventional method for isolation and purification chemical compounds from a medium. Well-known purification procedures include, but are not limited to, centrifugation or filtration, precipitation, ion exchange, chromatographic methods such as e.g. ion exchange chromatography or gel filtration chromatography, and crystallization methods.

The present invention thus provides a biochemical obtainable by a method as detailed herein.

### Certain Other Definitions

The term “mesophilic” as used herein in the context of a bacterium means that the bacterium grows best in moderate temperature with an optimum growth range from 20 to 45° C.

The term “methylotrophic” as used herein in the context of a bacterium means that the bacterium can use reduced one-carbon compounds, such as methanol, methane, formate, or methylamine, as the carbon source for their growth, and multi-carbon compounds that contain no carbon-carbon bonds, such as dimethyl ether and dimethylamine.

As used herein, a “polypeptide having glutamate decarboxylase (GAD) activity” or a “polypeptide which has glutamate decarboxylase (GAD) activity” means a polypeptide that catalyzes the reaction: L-glutamate<=>4-aminobutanoate+CO(2) (EC 4.1.1.15). Non-limiting examples of such polypeptides are provided in SEQ ID NOs: 1 to 10.

As used herein, a “polypeptide having α-ketoglutarate decarboxylase (KDC) activity“or a” polypeptide which has α-ketoglutarate decarboxylase (KDC) activity” means a polypeptide that catalyzes the reaction: 2-oxoglutarate<=>succinate semialdehyde+CO(2) (EC 4.1.1.71). Non-limiting examples of such polypeptides are provided in SEQ ID NOs: 39, 40, 41 and 42.

As used herein, a “polypeptide having GABA transaminase (GABA-TA) activity” or a “polypeptide which has GABA transaminase (GABA-TA) activity” means a polypeptide that catalyzes the reaction: 4-aminobutanoate+2-oxoglutarate<=>succinate semialdehyde+L-glutamate (EC 2.6.1.19). Non-limiting examples of such polypeptides are provided in SEQ ID NOs: 43 to 74.

As used herein, a “polypeptide having pyrrolidone synthase (PYS) activity” or a “polypeptide which has pyrrolidone synthase (PYS) activity” means a polypeptide that catalyzes the ring closing dehydration of γ-aminobutyrate. A non-limiting example of such polypeptide is provided in SEQ ID NO: 75, and is further described in, e.g, US 2016/0304922.

“Polypeptide” and “protein” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Nucleic acid” or “polynucleotide” are used interchangeably herein to denote a polymer of at least two nucleic acid monomer units or bases (e.g., adenine, cytosine, guanine, thymine) covalently linked by a phosphodiester bond, regardless of length or base modification.

“Recombinant” or “non-naturally occurring” when used with reference to, e.g., a host cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant bacterial cells expressing genes that are not found within the native (non-recombinant) form of the cell or express native genes that are otherwise expressed at a different level.

“Heterologous” or “exogenous” as used herein in the context of a gene or nucleic acid molecule refer to a gene or nucleic acid molecule (i.e. DNA or RNA molecule) that does not occur naturally as part of the genome of the bacterium in which it is present or which is found in a location or locations in the genome that differ from that in which it occurs in nature. Thus, a “heterologous” or “exogenous” gene or nucleic acid molecule is not endogenous to the bacterium and has been exogenously introduced into the microorganism. A “heterologous” gene or nucleic acid molecule DNA molecule may be from a different organism, a different species, a different genus or a different kingdom, as the host DNA.

“Heterologous” as used herein in the context of a polypeptide means that a polypeptide is normally not found in or made (i.e. expressed) by the host microorganism, but derived from a different organism, a different species, a different genus or a different kingdom.

As used herein, the term “ortholog” or “orthologs” refers to genes, nucleic acid molecules encoded thereby, i.e., mRNA, or proteins encoded thereby that are derived from a common ancestor gene but are present in different species.

“Substitution” or “substituted” refers to modification of the polypeptide by replacing one amino acid residue with another, for instance the replacement of a Serine residue with a Glycine or Alanine residue in a polypeptide sequence is an amino acid substitution. When used with reference to a polynucleotide, “substitution” or “substituted” refers to modification of the polynucleotide by replacing one nucleotide with another, for instance the replacement of a cytosine with a thymine in a polynucleotide sequence is a nucleotide substitution.

“Conservative substitution”, when used with reference to a polypeptide, refers to a substitution of an amino acid residue with a different residue having a similar side chain, and thus typically involves substitution of the amino acid in the polypeptide with amino acids within the same or similar class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having an aromatic side chain is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Non-conservative substitution”, when used with reference to a polypeptide, refers to a substitution of an amino acid in a polypeptide with an amino acid with significantly differing side chain properties. Non-conservative substitutions may use amino acids between, rather than within, the defined groups and affects (a) the structure of the peptide backbone in the area of the substitution (e.g., serine for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Expression” includes any step involved in the production of a polypeptide (e.g., encoded enzyme) including, but not limited to, transcription, post-transcriptional modification, translation, post-translational modification, and secretion.

As used herein, “vector” refers to a nucleic acid molecule capable of transporting another nucleic acid molecule to which it has been linked. One type of vector is a “plasmid”, which refers to a circular double stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of an exogenous nucleic acid molecule into a genome of a bacterium. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

As used herein, “promoter” refers to a sequence of DNA, usually upstream (5) of the coding region of a structural gene, which controls the expression of the coding region by providing recognition and binding sites for RNA polymerase and other factors which may be required for initiation of transcription. The selection of the promoter will depend upon the nucleic acid sequence of interest. A suitable “promoter” is generally one which is capable of supporting the initiation of transcription in a bacterium of the invention, causing the production of an mRNA molecule.

As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. A promoter sequence is “operably-linked” to a gene when it is in sufficient proximity to the transcription start site of a gene to regulate transcription of the gene.

“Percentage of sequence identity,” “% sequence identity” and “percent identity” are used herein to refer to comparisons between an amino acid sequence and a reference amino acid sequence. The “% sequence identity”, as used herein, is calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence.

“Reference sequence” or “reference amino acid sequence” refers to a defined sequence to which another sequence is compared. In the context of the present invention a reference amino acid sequence may, for example, be an amino acid sequence set forth in SEQ ID NO: 1.

As used herein, “derivative” of GABA refers to a compound derived from GABA by modification thereof. Non-limiting examples of a GABA “derivative” include 2-pyrrolidone, N-methyl-2-pyrrolidone and polybutyrolactam (Nylon 4).

As used herein, the term “about” means plus or minus 10% of the numerical value of the number with which it is being used.

Where a numerical limit or range is stated herein, the endpoints are included. Also, all values and sub ranges within a numerical limit or range are specifically included as if explicitly written out.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps or components but do not preclude the addition of one or more additional features, steps, components or groups thereof.

Having generally described this invention, a further understanding can be obtained by reference to certain specific examples, which are provided herein for purposes of illustration only, and are not intended to be limiting unless otherwise specified.

### EXAMPLES

### 1. Genetic Methods

### Example 1: Plasmid Preparation and Transformation

Plasmids contained a pBBR1 origin or replication, a kanamycin resistance or a rifampicin resistance cassette, and a lacO/trc promoter for controlling gene expression. Custom genes were inserted downstream of the promotor using HiFi assembly. Plasmids were constructed to express single genes, i.e. pABM36 was used to express the gene SEQ ID NO: 91. In some cases, plasmids were used to express two genes under the control of a single promoter. An additional RBS sequence was placed immediately downstream of the first gene and immediately upstream of the second genes. Examples include pABM45, which expresses the genes SEQ ID NO: 86 and SEQ ID NO: 87.

Plasmid DNA was prepared using the NEB HiFi 2× Master Mix according to manufacturer instruction. HiFi assemblies were transformed to Escherichia coli DH5a strains. Transformants were selected with 50 μg/mL kanamycin or 50 μg/mL rifampicin in solid and liquid 2TY medium. Plasmid DNA was extracted from overgrown liquid cultures using the GeneJet MiniPrep kit according to manufacturer instructions.

Plasmid DNA (e.g. pABM36) was transformed to ABME 3, ABME 5, ABME 6, and their derivative strains via electroporation. Transformations plated onto selective PM7 plates containing 20 g/L agar, 50 μg/mL kanamycin, or 5 Ng/mL rifampicin and incubated at 37° C. for 24-48 h.

All the plasmids that were prepared are listed in Table 1. The strains prepared by plasmid transformation are listed in Table 2.

TABLE 1
List of plasmids

| Plasmid | Purpose |
|---|---|
| pABM4 | Expression of GFP with gene sequence SEQ ID NO: 78 |
| | and protein sequence SEQ ID NO: 76 |
| pABM28 | Co-expression of mCherry with gene |
| | sequence SEQ ID NO: 79 and protein |
| | sequence SEQ ID NO: 77, and GPF with gene sequence |
| | SEQ ID NO: 78 and protein sequence SEQ ID NO: 76 |
| pABM34 | Expression of Lbr GAD 4AAmut with gene sequence |
| | SEQ ID NO: 85 and protein sequence SEQ ID NO: 38 |
| pABM35 | Expression of Lbr GAD with gene sequence |
| | SEQ ID NO: 82 and protein sequence SEQ ID NO: 10 |
| pABM36 | Expression of Eco GAD E89Q Δ14C with gene |
| | sequence SEQ ID NO: 91 and protein sequence |
| | SEQ ID NO: 90 |
| pABM37 | Expression of Sth GAD E84Q Δ14C with gene |
| | sequence SEQ ID NO: 84 and protein sequence |
| | SEQ ID NO: 92 |
| pABM44 | Co-expression of Egr KDC with gene sequence |
| | SEQ ID NO: 86 and protein sequence SEQ ID NO: 39, |
| | and Eco GABA-TA with gene sequence SEQ ID NO: |
| | 88 and protein sequence SEQ ID NO: 43 |
| pABM45 | Co-expression of Scc KDC with gene sequence |
| | SEQ ID NO: 87 and protein sequence SEQ ID NO: 40, |
| | and Eco GABA-TA with gene sequence SEQ ID NO: |
| | 88 and protein sequence SEQ ID NO: 43 |
| pABM TH1 | Expression of Sai PYS with gene |
| | sequence SEQ ID NO: 89 and protein |
| | sequence SEQ ID NO: 75 |
| pABM TH2 | Co-expression of Sai PYS with gene sequence |
| | SEQ ID NO: 89 and protein sequence SEQ ID NO: 75 |
| | and Eco GAD E89Q Δ14C with gene sequence SEQ |
| | ID NO: 91 and protein sequence SEQ ID NO: 90 |

TABLE 2
List of strains prepared by plasmid transformation

| Strain | Genotype/parent strain | Plasmid |
|---|---|---|
| ABME 5 | Wild-type Methylobacillus | / |
| | glycogenes DSM 5685 | |
| ABME 23 | ABME 5 | pABM4 |
| ABME 27 | ABME 5 | pABM28 |
| ABME 32 | ABME 5 | pABM 34 |
| ABME 33 | ABME 5 | pABM 35 |
| ABME 34 | ABME 5 | pABM 36 |
| ABME 35 | ABME 5 | pABM 37 |
| ABME 99 | ABME 5 | pABM44 |
| ABME 101 | ABME 5 | pABM45 |
| ABME 6 | Wild-type Methylobacillus | / |
| | flagellatus DSM 6875 | |
| ABME 25 | ABME 6 | pABM4 |
| ABME 65 | ABME 6 | pABM28 |
| ABME 38 | ABME 6 | pABM34 |
| ABME 39 | ABME 6 | pABM35 |
| ABME 40 | ABME 6 | pABM36 |
| ABME 41 | ABME 6 | pABM37 |
| ABME 90 | ABME 6 | pABM44 |
| ABME 104 | ABME 6 | pABM45 |
| ABME 6TH2 | ABME 126 | pABM TH1 |
| ABME 6TH3 | ABME 6 | pABM TH2 |

### Example 2: Gene Integration

GABA producing genes were integrated into the genome of ABME 6, or ABME 6-derived strains. The genome edits were achieved by introducing a linear DNA fragment consisting of the gene of interest and a kanamycin or chloramphenicol resistance cassette flanked by 2 kb long sequences upstream and downstream of the integration sites. DNA fragments were transformed into ABME 6 by electroporation. Transformations were plated onto selective PM7 plates containing 20 g/L agar, 50 μg/mL kanamycin, or 500 μg/mL thiamphenicol and incubated at 37° C. for 24-48. Correct clones were confirmed by colony PCR.

Strain ABME 126 was generated by integrating the gene with SEQ ID NO: 91 by replacing the AMBE 6 epsD-G genes.

Strain ABME 6TH1 was generated by integrating the gene with SEQ ID NO: 87 by replacing the genes Mfla_1268-1280, and by integrating the gene with SEQ ID NO: 88 by replacing the AMBE 6 epsD-G genes.

The strains prepared by genome integration are listed in Table 3.

TABLE 3
List of strains prepared with genomic integration.

| Strain | Genotype/parent strain | Plasmid |
|---|---|---|
| ABME 6 | Wild-type Methylobacillus flagellatus DSM 6875 | / |
| ABME 126 | epsD-G::Eco_gad_E89Q-kanR/ABME 6 | / |

### Example 3: Expression of Fluorescent Proteins in the Genera Metylobacillus and Methylobacterium

To test the capacity of methylotrophs from the genera Methylobacillus and Methylobacterium, GFP and mCherry were expressed from plasmid. GFP was expressed alone (ABME 23, ABME 25) or co-expressed in combination with mCherry (ABME 27 and ABME 65). The strains and plasmids were prepared as described in Example 1.

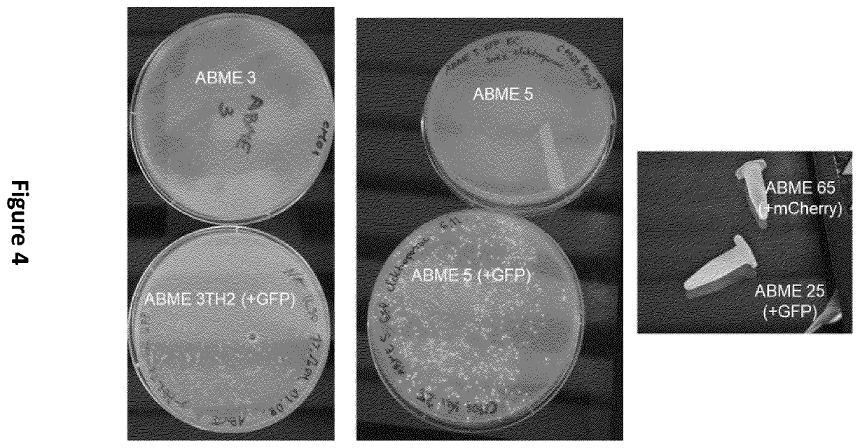

To assess observe GFP and RFP fluorescence, fresh single colonies were grown overnight on PM7+50 μg/mL kanamycin agar plates. Single colonies were resuspended in 25 mL PM7 medium+25 μg/mL kanamycin in baffled 250 mL shake flasks, and incubated overnight at 200 rpm. Next day, 50 mL PM7 medium+25 μg/mL kanamycin in baffled 250 mL shake flasks was inoculated with 5 mL overnight culture. After 4 h of growth, 0.5 g/L IPTG was added to induce gene expression. The medium was supplemented with 30 g/L glutamate. All ABME 3-derived strains and ABME 5-derived strains were cultivated at 30° C., and all ABME 6-derived strains were cultivated at 37° C. Cultures were analyzed after 24 h of growth. The GFP and RFP expression was sufficiently high to detect visually when plates or cell pellets were exposed to UV light (FIG. 4).

### II. pH-Independent GABA Production Via a GAD Enzyme

### Example 4: Functional Expression of Three GAD Enzymes in ABME 6—In Vitro Test

Three GAD enzymes were tested in vitro to assess their capacity for functional GABA production when expressed in ABME 6-derived strains (ABME 38, ABME 39, and ABME40).

GAD enzymes were taken from E. coli and Lactobacillus brevis (Lbr). Eco GAD had an amino acid substitution, where glutamate at position 89 was replaced by glutamine (SEQ ID NO: 90) and 14 AA removed from the C-terminus. Lbr GAD was tested both as a wild-type enzyme (SEQ ID NO: 10), as well as a mutated version, where threonine at position 17 was substituted with isoleucine, aspartate at position 94 was substituted with glycine, glutamate at position 213 was substituted with serine, and glutamine at position 346 was replaced with histidine (SEQ ID NO: 38). The GAD variants were individually expressed in strains ABME 39, ABME 38, and ABME 40 which were obtained by transforming ABME 6 with the plasmids the plasmids pABM34, pABM35, and pABM36 as described in Example 1.

To prepare the cell lysates, fresh single colonies were grown overnight on PM7+50 μg/mL kanamycin agar plates at 37° C. Single colonies were resuspended in 25 mL PM7 medium+25 μg/mL kanamycin in baffled 250 mL shake flasks, and incubated overnight at 37° C. and 200 rpm. Next day, 25 mL PM7 medium+25 μg/mL kanamycin in baffled 250 mL shake flasks was inoculated with 2.5 mL overnight culture and grown to an OD600=1.0 at 37° C. and 200 rpm. The cultures were induced with 0.5 g/L IPTG for 3 h and then centrifuged to concentrate the cells. The cell pellet was lysed by three freeze-thaw cycles at −80° C. The cell lysates were clarified by centrifugation at 4500 rpm and 4° C.

The clarified cell lysates were used to determine the in vitro activity of the three GAD variants in individual GAD enzyme assays reaction mixtures (Table 6). The reactions were incubated for 4 h at 37° C. The resulting GABA was measured by HPLC.

The HPLC method was set-up on Thermo Surveyor+HPLC system, equipped with a PDA detector and coupled to Corona+charged aerosol detector. Chromatographic separation was based on an AkzoNobel Kromasil 100-5 C18 HPLC column, 250×4.6 mm, with 5 um particle size, thermostated at 60° C. in the column oven. Mobile phase A was 0.09% aqueous solution of heptafluorobutyric acid (HFBA) and mobile phase B was methanol (MeOH), with flow 1.0 ml/min on column with the gradient program as described in Table 4. Samples were injected with 10% MeOH vs 90% aqueous solution of % 0.05 TFA, and analytes are detected with CAD set at 36 psi nitrogen, within 100 pA range.

TABLE 4
Gradient used in the HPLC method for GABA detection.

| Time/min: | % A | % B |
|---|---|---|
| 0.0 | 85 | 15 |
| 4.5 | 85 | 15 |
| 4.6 | 1 | 99 |
| 6.5 | 1 | 99 |
| 6.6 | 85 | 15 |
| 15.0 | 85 | 15 |

TABLE 5
Composition of PM7 medium

| Compound | Per 1 L |
|---|---|
| MeOH | 10 g |
| KH2PO4 | 0.65 g |
| NA2HPO4 | 1.05 g |
| MgSO4 * 7H2O | 0.1 g |
| NH4Cl | 0.2 g |
| (NH4)2SO4 | 2 g |
| TE1 1000X | 1 mL |
| d-Biotin | 0.1 mg |
| Thiamine*HCl | 0.1 mg |
| Riboflavin | 0.1 mg |
| Pyridoxine*HCl | 0.1 mg |
| Pantothenate | 0.1 mg |
| Nicotinamide | 0.1 mg |
| p-Aminobenzoic acid | 0.02 mg |
| Folic acid | 0.01 mg |
| Vitamin B12 | 0.01 mg |
| Lipoic acid | 0.01 mg |
| CaCl2 * 2H2O | 3.3 mg |
| FeSO4 * 7H2O | 1.3 mg |
| MnSO4 * H2O | 0.1 mg |
| ZnSO4 * 7H2O | 0.13 mg |
| CuSO4 * 5H2O | 0.04 mg |
| Na2MoO4 * 2H2O | 0.04 mg |
| CoCl2 * 6H2O | 0.04 mg |
| H3BO3 | 0.03 mg |
| pH | 7.0 |
| Agar (for solid media) | 20 g |

TABLE 6
GAD enzyme assay reaction

| Component | Concentration |
|---|---|
| Ammonium-Acetate buffer, pH 5.5 | 0.5 M |
| Glutamate | 5 g/L |
| Pyridoxal-phosphate | 750 UM |
| Cell lysate | 100 ug |
| MQ water | To 1 mL |

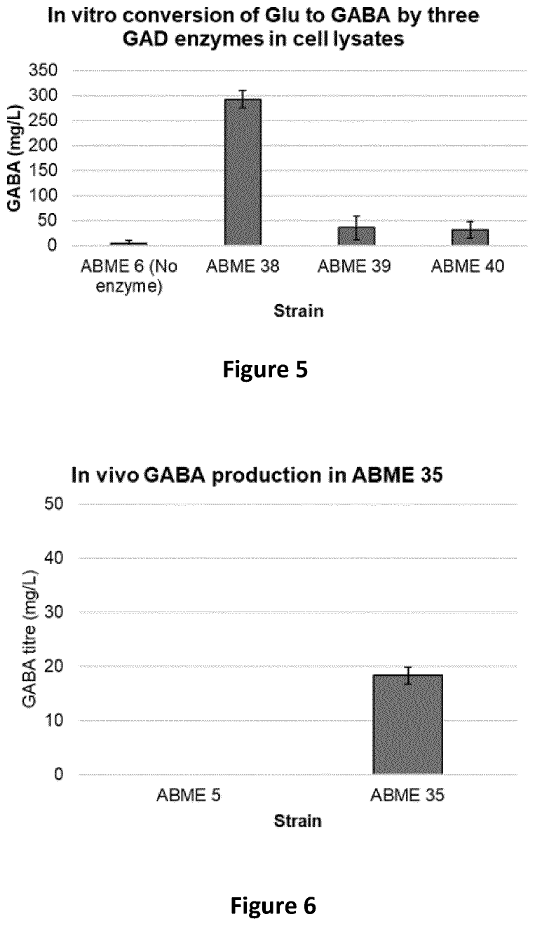

GABA formation was detected in all lysates that contained the GAD enzyme. ABME 38 cell lysate formed the most GABA, 239+18 mg/L, while the lysates of ABME 39 and ABME 40 formed approximately 30 mg/L GABA (FIG. 5). The in vitro GAD activity observed in strains ABME 38, ABME 39, and ABME 40 demonstrated for the first time that expression of GAD in Methylobacillus flagellatus is possible.

### Example 5: Sth GAD Enzyme in ABME 5

The Sulfobacillus thermosulfidooxidans (Sth) GAD enzyme was tested in vivo to assess its capacity for GABA production when expressed from plasmid in the ABME 5-derived strain ABME 34 with external glutamate supplementation.

The GAD enzyme was taken from Sulfobacillus thermosulfidooxidans (Sth) with an amino acid substitution, where glutamate at position 84 was replaced by glutamine, and the C-terminal 14 amino acids deleted (SEQ ID NO: 84). The enzyme was expressed from plasmid pABM37 as described in Example 1.

Strain ABME 35 was obtained by transforming ABME 5 with the plasmid pABM37 as described in Example 1.

To assess in vivo GABA production by Sth GAD in shake flasks, fresh single colonies were grown overnight on PM7+50 μg/mL kanamycin agar plates. Single colonies were resuspended in 25 mL PM7 medium+25 μg/mL kanamycin in baffled 250 mL shake flasks, and incubated overnight at 200 rpm. Next day, 50 mL PM7 medium+25 μg/mL kanamycin in baffled 250 mL shake flasks was inoculated with 5 mL overnight culture. After 4 h of growth, 0.5 g/L IPTG was added to induce gene expression. The medium was supplemented with 30 g/L glutamate. All ABME 5-derived strains were cultivated at 30° C. Cultures were sampled after 24 h of growth. GABA was measured by HPLC.

ABME 35 produced 18±2 mg/L GABA (FIG. 6). No GABA formation was detected in the negative controls (ABME 5).

### Example 6: Direct GABA Production from Methanol with Methylobacillus in Shake Flasks by GAD Expression from a Plasmid

Two GAD enzymes were further tested in vivo to assess their capacity for GABA production from methanol when expressed from plasmid in ABME 5-derived strain (ABME 32 and ABME 34) and ABME 6-derived strains (ABME 38 and ABME 40). In this example, no external glutamate was supplemented.

GAD enzymes were taken from E. coli and Lactobacillus brevis (Lbr). Eco GAD had an amino acid substitution, where glutamate at position 89 was replaced by glutamine, and the C-terminal 14 amino acids deleted (SEQ ID NO: 90). Lbr GAD was tested both as a wild-type enzyme (SEQ ID NO: 10), as well as a mutated version, where threonine at position 17 was substituted with isoleucine, aspartate at position 94 was substituted with glycine, glutamate at position 213 was substituted with serine, and glutamine at position 346 was replaced with histidine (SEQ ID NO: 38).

Strains ABME 32, ABME 34, ABME 38, and ABME 40 were obtained as described in Example 1. In vivo GABA production by the strains was tested in shake flasks, as described in Example with the omission of external glutamate. The cultures were sampled after 24 h and 48 h of growth. GABA production was measured by HPLC as described in Example.

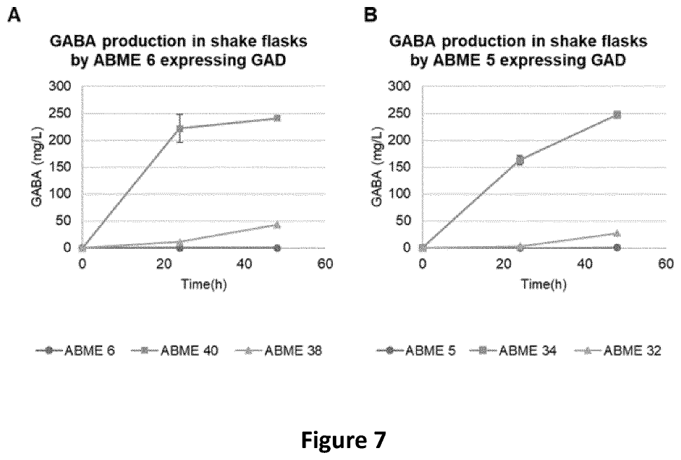

All four strains (ABME 32, ABME 34, ABME 38, and ABME 40) expressing GAD enzymes formed GABA directly from methanol without external glutamate addition (FIG. 7AB). ABME 5 and ABME 6 did not express GAD enzymes and were used as negative controls. The strains ABME 34 and ABME 40 formed the most GABA (approximately 250 mg/L). This was surprising, as the in vitro test of four GAD enzymes (Example 4) showed that the Eco GAD E89Q A14C enzyme is less efficient compared to the Lbr GAD 4AAmut. In comparison, the strains expressing Lbr GAD 4AAmut (ABME 32 and ABME 38) formed less than 50 mg/L GABA.

This experiment is the first known example of direct GABA synthesis from methanol in any methylotroph without a pH-drop, which was reported so far in literature. To achieve this, it was necessary to use a mesophilic methylotroph to express the GAD enzymes, which was not expected.

### Example 7: Direct GABA Production from Methanol with Methylobacillus in Reactors by GAD Expression from a Plasmid

The best performing strain, ABME 40 was tested in 5 L and 20 L pH-controlled bioreactors to prove that GABA production was independent from pH and extend the GABA production phase. The process was oxygen limited with gradual methanol feeding.

The strain ABME 40 was prepared as described in Example 1. The bioreactors contained PM3 medium+25 μg/mL kanamycin and were inoculated with an 8% (5 L reactors) or 1% (20 L reactors) inoculum of ABME 40 with an OD600=1.0. pH was maintained at 7.0 by automatic addition of NH₄OH. Methanol concentrations were measured online and were kept at approximately 5 g/L throughout the fermentation by automatic addition of pure methanol. Reactor temperature was maintained at 37° C. throughout the process. The reactor was kept oxygen limited by flushing with 2 VVM (5 L reactors) or 1VVM (20 L reactors) air while maintaining the pO₂ value at 0.0%. 0.5 g/L IPTG was added to the reactors after 19 h to induce GAD expression. Reactors were sampled regularly throughout the process. GABA and methanol were analyzed by HPLC as described in Example. Growth was monitored by measuring OD600.

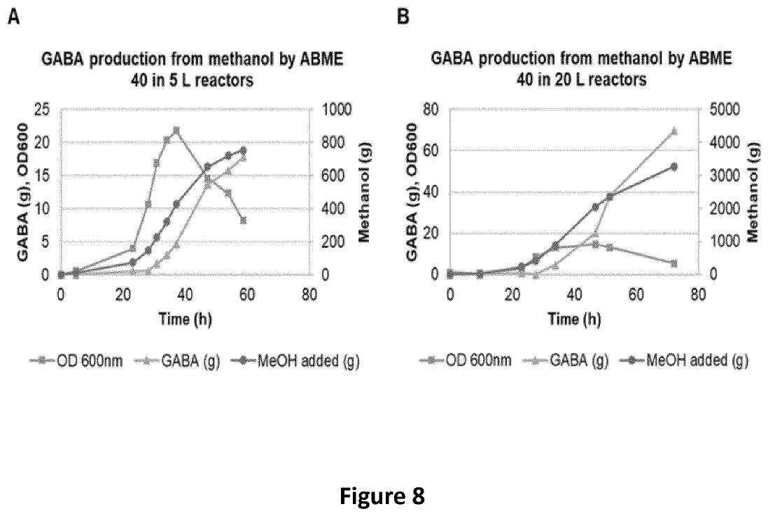

The strain ABME 40 produced GABA directly from methanol at pH 7.0 in absence of a pH-drop in both 5 L and 20 L bioreactors (FIGS. 8A and B, respectively). The strain produced 17.8 g of GABA in 5 L bioreactors, and 69.8 g GABA in 20 L reactors. These amounts were significantly higher compared to the amounts reached in shake flasks (Example 6). They were also surprising since the parent strain (ABME 6) is not known as a natural producer of glutamate, which is a precursor for GABA formation. This experiment further demonstrated the feasibility of pH-independent direct GABA synthesis from methanol without a pH drop using the stains disclosed in this invention.

TABLE 7
Composition of the PM3 medium

| Compound | Per 1 L |
|---|---|
| KH2PO4 | 1.95 g |
| NA2HPO4 | 3.15 g |
| MgSO4 * 7H2O | 0.1 g |
| (NH4)2SO4 | 2 g |
| d-Biotin | 0.1 mg |
| Thiamine*HCl | 0.1 mg |
| Riboflavin | 0.1 mg |
| Pyridoxine*HCl | 0.1 mg |
| Pantothenate | 0.1 mg |
| Nicotinamide | 0.1 mg |
| p-Aminobenzoic acid | 0.02 mg |
| Folic acid | 0.01 mg |
| Vitamin B12 | 0.01 mg |
| Lipoic acid | 0.01 mg |
| CaCl2 * 2H2O | 33 mg |
| FeSO4 * 7H2O | 13 mg |
| MnSO4 * H2O | 1 mg |
| ZnSO4 * 7H2O | 1.3 mg |
| CuSO4 * 5H2O | 0.4 mg |
| Na2MoO4 * 2H2O | 0.4 mg |
| CoCl2 * 6H2O | 0.4 mg |
| H3BO3 | 0.3 mg |
| pH | 7.0 |

### Example 8: Direct GABA Production from Methanol with Methylobacillus in Shake Flasks by Genomic GAD Expression

The best performing GAD gene, Eco GAD E89Q A14C (SEQ ID NO: 91) was integrated in the strain ABME 126, as described in Example. The genomic integration of GAD is beneficial since plasmid-based gene expression is not commercially viable in the long term. In vivo GABA production by ABME 126 was assessed in shake flasks, as described in Example. The cultures were sampled after 24 h of growth. GABA production was measured by HPLC, as described in Example 3.

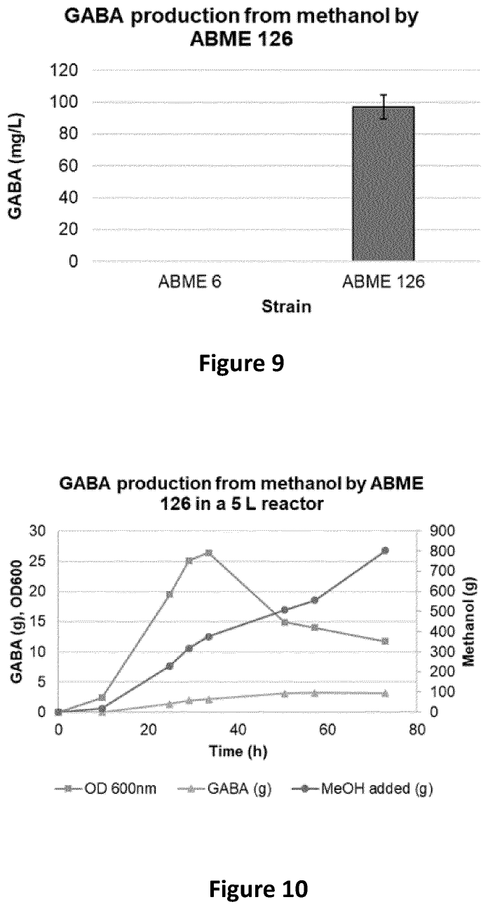

The strain ABME 126 produced almost 97±7 mg/L GABA, while the negative control strain ABME 6 did not produce any GABA (FIG. 9). This experiment demonstrated that GABA formation from methanol is also possible when the Eco gad E89Q A14C gene (SEQ ID NO: 91) was expressed as a single copy from the genome. It was unexpected and surprising that the native epsD-G promoter would be sufficiently strong to result in GABA production without optimization.

### Example 9: Direct GABA Production from Methanol with Methylobacillus in Reactors by Genomic GAD Expression

The strain ABME 126, which was prepared as described in Example 1 was tested in 5 L bioreactors to prove that GABA production was independent from pH and extend the GABA production phase. The process was carbon limited with gradual methanol feeding to extend the GABA production phase.

The bioreactors contained PM3 medium and were inoculated with an 8% (5 L reactors) inoculum of ABME 126 with an OD600=1.0. pH was maintained at 7.0 by automatic addition of NH4OH. The reactor temperature was maintained at 37° C. throughout the process. The reactor was kept carbon-limited by controlling the automatic addition of 50% methanol at a rate that maintained pO2-value at approximately 30%. Methanol concentration in the liquid were measured online and were zero throughout the process. The reactor was sampled regularly. GABA and methanol were analyzed by HPLC. Growth was monitored by measuring OD600.

The strain ABME 126 formed 3.2 g GABA in pH-controlled bioreactors (FIG. 10), which was significantly higher compared to the amount produced in shake flasks (Example 8). This experiment provides further evidence that genomic expression of the Eco_gad E89Q A14C (SEQ ID NO: 91) gene enables pH-independent GABA production from methanol.

### III. pH-Independent GABA Production Via an Alternative Pathway

### Example 10: Identification of a Potential Novel Pathway for GABA Production from Methanol

In this example, the identification of a theoretical novel GABA-producing pathway (GAD-bypass) is disclosed. The benefit of this alternative pathway is that does not use the GAD enzyme, which is pH-dependent in its native from.

The pathway was identified by reversing the order of reactions that convert α-ketoglutarate to GABA. In the conventional GABA-forming pathway described in the Examples above, α-ketoglutarate is first converted to glutamate via a reductive amination reaction, catalyzed by glutamate dehydrogenase (GDH). Next, glutamate is decarboxylated to GABA by removing the α-carbon-adjacent carbonyl group. The general reaction sequence in the conventional GABA pathway is thereby amination, followed by decarboxylation (Example 10A).

We investigated whether the reaction sequence could be reversed, i.e. decarboxylation that is followed by amination. For this pathway to function, α-ketoglutarate would first have to be decarboxylated by removing the keto-adjacent carbonyl group. This would yield succinate semialdehyde, which would then have to be aminated at the aldehyde group to form GABA.

After establishing the necessary chemical conversion steps needed to convert α-ketoglutarate to GABA by decarboxylation followed by amination, we examined whether enzymes that catalyze these reactions exist. Surprisingly, we were able to identify enzymes that perform both required reactions, and are theoretically able to form GABA form α-ketoglutarate independently from the GAD enzyme.

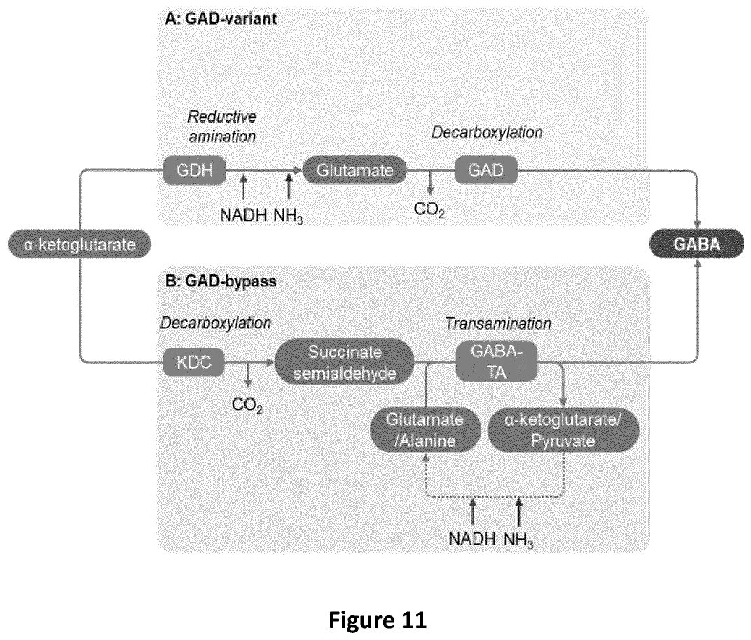

The decarboxylation of α-ketoglutarate to succinate semialdehyde can be performed α-ketoglutarate decarboxylase (KDC), an enzyme used in some rare variants of the TCA cycle by Euglena gracilis (SEQ ID NO: 39) and Synechococcus sp. (SEQ ID NO: 40) The next step of succinate semialdehyde amination could then theoretically be catalyzed by GABA transaminase (GABA-TA). Unexpectedly, this enzyme is usually involved in GABA degradation. However, the AGrO of the reaction is near 0, indicating that the reaction may be reversible, and could catalyze the formation of GABA as well. The GABA-TA enzyme is present in many organisms, including E. coli (SEQ ID NO: 43). The transamination reaction transfers the amino group from glutamate or alanine to succinate semialdehyde and forms GABA and α-ketoglutarate or pyruvate in the process (FIG. 11B).

The identification of the novel GAD-bypass provides a previously unknown and unexpected pathway to form GABA from methanol in a pH-independent manner.

### Example 11: Direct GABA Production from Methanol with Methylobacillus in Shake Flasks by Expression of the GAD-Bypass from a Plasmid

To test whether the GAD-bypass is able to form GABA from methanol in vivo, two KDC enzymes from Euglena gracilis (Egr, SEQ ID NO: 86) and Synechococcus sp. (Scc, SEQ ID NO: 87) PCC7002 were expressed in combination with the E. coli (Eco) GABA-TA (gabT gene, SEQ ID NO: 88). SEQ ID NO: 86 and SEQ ID NO: 87 were codon harmonized for expression in ABME 6. The genes were expressed in strains ABME 99, ABME 101, ABME 90, and ABME 104, respectively, as described in Example 1.

In vivo GABA production by the strains was assessed as described in Example 6. The cultures were sampled after 24 h of growth. GABA production was measured by HPLC, as described in Example 4.

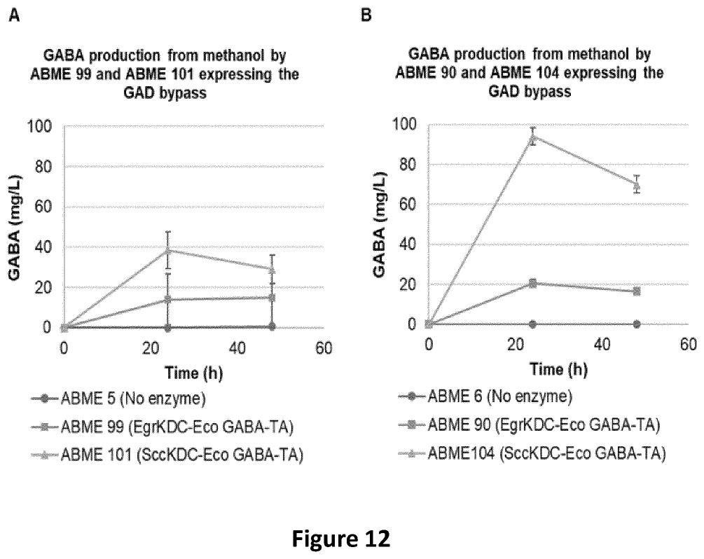

The strains ABME 99, ABME 101, ABME 90, and ABME 104 were all able to produce GABA from methanol in shake flasks (FIG. 12). This confirmed that the novel GAD-bypass pathway is indeed able to form GABA from methanol independently of the GAD enzyme. The pathway variant utilizing the SccKDC (SEQ ID NO: 40) and Eco GABA-TA (SEQ ID NO: 43) enzymes was more efficient compared to the EgrKDC (SEQ ID NO: 39) variant. The ABME 6-derived strain ABME 104 formed the most GABA via the GAD bypass in this example (94±4 mg/L, FIG. 12B).

GABA production by the GAD-bypass was theoretically possible, but still surprising since neither the KDC nor GABA-TA enzymes are native to Methylobacillus glycogenes or Methylobacillus flagellatus. It was surprising that they would be able to produce GABA immediately without any optimization of enzyme activity. It was particularly surprising that the GABA-TA enzyme, which normally catalyzes GABA degradation, was able to form GABA as part of the GAD bypass.

This experiment demonstrates for the first time a novel pathway for GABA formation from methanol in a GAD- and pH-independent manner.

### Example 12: Direct GABA Production from Methanol with Methylobacillus in Reactors by Expression of the GAD-Bypass from a Plasmid

The strain ABME 104, which was prepared as described in Example 1 was tested in 5 L bioreactors to prove that GABA production was independent from pH and extend the GABA production phase. The process was oxygen limited with gradual methanol feeding. The process was performed as described in Example 7 using the strain ABME 104.

In pH-controlled bioreactors, the strain ABME 104 expressing the GAD bypass was able to form 0.8 g GABA directly from methanol. The GABA formation occurred at pH 7.0, confirming that the GAD-bypass is able to produce GABA in a pH independent manner, and represents a novel pathway for GABA production from methanol.

### LIST OF REFERENCES CITED IN THE DESCRIPTION

Irla, M., Nmrdal, I., Brautaset, T., Wendisch, V. F., 2017. Methanol-based γ-aminobutyric acid (GABA) production by genetically engineered Bacillus methanolicus strains. Ind. Crops Prod. 106, 12-20. https://doi.org/https://doi.org/10.1016/j.indcrop.2016.11.050
Pennacchietti E, Lammens T M, Capitani G, Franssen M C, John R A, Bossa F, De Biase D., 2009. Mutation of His465 alters the pH-dependent spectroscopic properties of Escherichia coli glutamate decarboxylase and broadens the range of its activity toward more alkaline pH. J Biol Chem. 284(46):31587-96. doi: 10.1074/jbc.M109.049577.
Thu Ho, N. A., Hou, C. Y., Kim, W. H., Kang, T. J., 2013. Expanding the active pH range of Escherichia coli glutamate decarboxylase by breaking the cooperativeness. J. Biosci. Bioeng. https://doi.org/10.1016/j.jbiosc.2012.09.002
Yu, K., Lin, L., Hu, S., Huang, J., Mei, L., 2012. C-terminal truncation of glutamate decarboxylase from Lactobacillus brevis CGMCC 1306 extends its activity toward near-neutral pH. Enzyme Microb. Technol. 50, 263-269. https://doi.org/10.1016/j.enzmictec.2012.01.010
Zhang, J., Kao, E., Wang, G., Baidoo, E. E. K., Chen, M., Keasling, J. D., 2016. Metabolic engineering of Escherichia coli for the biosynthesis of 2-pyrrolidone. Metab. Eng. Commun. https://doi.org/10.1016/j.meteno.2015.11.001

1. A mesophilic, methylotrophic bacterium capable of expressing a polypeptide, which has glutamate decarboxylase (GAD) activity and is catalytically active at a pH above 5.5.

2-15. (canceled)

16. The bacterium according to claim 1, wherein the polypeptide is catalytically active at a pH in the range of pH 6 to pH 8.

17. The bacterium according to claim 1, wherein the polypeptide is a GAD mutant comprising in its amino acid sequence at least one amino acid substitution compared to the wild type GAD enzyme from which it is derived rending the mutant active at a pH above 5.5.

18. The bacterium according to claim 17, wherein the amino acid substitution is in a position corresponding to position 89 and/or position 465 of SEQ ID NO: 1.

19. The bacterium according to claim 1, wherein the polypeptide is a GAD mutant having 2 to 20 amino acids deleted from the C-terminus compared to the wild type GAD enzyme from which it is derived.

20. The bacterium according to claim 1, wherein the polypeptide is selected from any one of the following:
a) a polypeptide having glutamate decarboxylase (GAD) activity which comprises an amino acid sequence having at least 70% sequence identity with SEQ ID NO: 1 and comprising an amino acid substitution at a position corresponding to position 89 and/or position 465 in SEQ ID NO: 1;
b) a polypeptide having glutamate decarboxylase (GAD) activity which comprises an amino acid sequence having at least 70% sequence identity with SEQ ID NO: 2 and comprising an amino acid substitution at a position corresponding to position 84 and/or position 457 in SEQ ID NO: 2;
c) a polypeptide having glutamate decarboxylase (GAD) activity which comprises an amino acid sequence having at least 70% sequence identity with SEQ ID NO: 3 and comprising an amino acid substitution at a position corresponding to position 93 and/or position 467 in SEQ ID NO: 3;
d) a polypeptide having glutamate decarboxylase (GAD) activity which comprises an amino acid sequence having at least 70% sequence identity with SEQ ID NO: 4 and comprising an amino acid substitution at a position corresponding to position 90 and/or position 467 in SEQ ID NO: 4;
e) a polypeptide having glutamate decarboxylase (GAD) activity which comprises an amino acid sequence having at least 70% sequence identity with SEQ ID NO: 5 and comprising an amino acid substitution at a position corresponding to position 85 and/or position 462 in SEQ ID NO: 5;
f) a polypeptide having glutamate decarboxylase (GAD) activity which comprises an amino acid sequence having at least 70% sequence identity with SEQ ID NO: 6 and comprising an amino acid substitution at a position corresponding to position 89 and/or position 465 in SEQ ID NO: 6;
g) a polypeptide having glutamate decarboxylase (GAD) activity which comprises an amino acid sequence having at least 70% sequence identity with SEQ ID NO: 7 and comprising an amino acid substitution at a position corresponding to position 89 and/or position 466 in SEQ ID NO: 7;
h) a polypeptide having glutamate decarboxylase (GAD) activity which comprises an amino acid sequence having at least 70% sequence identity with SEQ ID NO: 8 and comprising an amino acid substitution at a position corresponding to position 89 and/or position 466 in SEQ ID NO: 8;
i) a polypeptide having glutamate decarboxylase (GAD) activity which comprises an amino acid sequence having at least 70% sequence identity with SEQ ID NO: 9 and comprising an amino acid substitution at a position corresponding to position 89 and/or position 463 in SEQ ID NO: 9;
j) a polypeptide having glutamate decarboxylase (GAD) activity which comprises an amino acid sequence having at least 70% sequence identity with SEQ ID NO: 10 and comprising an amino acid substitution at a position corresponding to position 17, position 91, position 94, position 213, position 346 and/or position 468 in SEQ ID NO: 10; or
k) a polypeptide having glutamate decarboxylase (GAD) activity which comprises an amino acid sequence having at least 70% sequence identity with SEQ ID NO: 1, 2, 3, 4, 5, 6, 7, 8, 9 or 10, wherein 2 to 20 amino acids are deleted from the C-terminus.

21. The bacterium according to claim 1, wherein the polypeptide is selected from any one of the following:
aa) a polypeptide having glutamate decarboxylase (GAD) activity, which comprises the amino acid sequence of SEQ ID NO: 11, 12 or 13 or an amino acid sequence corresponding to SEQ ID NO: 11, 12 or 13, wherein 1 to 40 amino acids are substituted with the proviso that the amino acid at position 89 is not E and/or the amino acid at position 465 is not H, and/or the amino acid at position 465 is A;
bb) a polypeptide having glutamate decarboxylase (GAD) activity, which comprises the amino acid sequence of SEQ ID NO: 14, 15 or 16 or an amino acid sequence corresponding to SEQ ID NO: 14, 15 or 16, wherein 1 to 40 amino acids are substituted with the proviso that the amino acid at position 84 is not E and/or the amino acid at position 457 is not H, and/or the amino acid at position 457 is A;
cc) a polypeptide having glutamate decarboxylase (GAD) activity, which comprises the amino acid sequence of SEQ ID NO: 17, 18 or 19 or an amino acid sequence corresponding to SEQ ID NO: 17, 18 or 19, wherein 1 to 40 are substituted with the proviso that the amino acid at position 93 is not E and/or the amino acid at position 467 is not H, and/or the amino acid at position 467 is A;
dd) a polypeptide having glutamate decarboxylase (GAD) activity, which comprises the amino acid sequence of SEQ ID NO: 20, 21 or 22 or an amino acid sequence corresponding to SEQ ID NO: 20, 21 or 22, wherein 1 to 40 are substituted with the proviso that the amino acid at position 90 is not E and/or the amino acid at position 467 is not H, and/or the amino acid at position 467 is A;
ee) a polypeptide having glutamate decarboxylase (GAD) activity, which comprises the amino acid sequence of SEQ ID NO: 23, 24 or 25 or an amino acid sequence corresponding to SEQ ID NO: 23, 24 or 25, wherein 1 to 40 are substituted with the proviso that the amino acid at position 85 is not E and/or the amino acid at position 462 is not H, and/or the amino acid at position 462 is A;
ff) a polypeptide having glutamate decarboxylase (GAD) activity, which comprises the amino acid sequence of SEQ ID NO: 26, 27 or 28 or an amino acid sequence corresponding to SEQ ID NO: 26, 27 or 28, wherein 1 to 40 amino acids are substituted with the proviso that the amino acid at position 89 is not E and/or the amino acid at position 465 is not H, and/or the amino acid at position 465 is A;
gg) a polypeptide having glutamate decarboxylase (GAD) activity, which comprises the amino acid sequence of SEQ ID NO: 29, 30 or 31 or an amino acid sequence corresponding to SEQ ID NO: 29, 30 or 31, wherein 1 to 40 are substituted with the proviso that the amino acid at position 89 is not E and/or the amino acid at position 466 is not H, and/or the amino acid at position 466 is A;
hh) a polypeptide having glutamate decarboxylase (GAD) activity, which comprises the amino acid sequence of SEQ ID NO: 32, 33 or 34 or an amino acid sequence corresponding to SEQ ID NO: 32, 33 or 34, wherein 1 to 40 are substituted with the proviso that the amino acid at position 89 is not E and/or the amino acid at position 466 is not H, and/or the amino acid at position 466 is A;
ii) a polypeptide having glutamate decarboxylase (GAD) activity, which comprises the amino acid sequence of SEQ ID NO: 35, 36 or 37 or an amino acid sequence corresponding to SEQ ID NO: 35, 36 or 37, wherein 1 to 40 are substituted with the proviso that the amino acid at position 89 is not E and/or the amino acid at position 463 is not H, and/or the amino acid at position 463 is A;
jj) a polypeptide having glutamate decarboxylase (GAD) activity, which comprises the amino acid sequence of SEQ ID NO: 38 or an amino acid sequence corresponding to SEQ ID NO: 38, wherein 1 to 40 amino acids are substituted with the proviso that the amino acid at position 17 is not T, the amino acid at position 94 is not D, the amino acid at position 213 is not E and/or the amino acid at position 346 is not Q, the amino acid at position 94 is G, the amino acid at position 213 is S and/or the amino acid at position 346 is H; or
kk) a polypeptide having glutamate decarboxylase (GAD) activity, which comprises an amino acid sequence having at least 70% sequence identity with any one of SEQ ID NOs: 11 to 38, wherein 2 to 20 amino acids are deleted from the C-terminus.

22. A mesophilic, methylotrophic bacterium capable of expressing:
a) a polypeptide having α-ketoglutarate decarboxylase (KDC) activity, and
b) a polypeptide having GABA transaminase (GABA-TA) activity.

23. The bacterium according to claim 22, wherein the polypeptide having KDC activity comprises the amino acid sequence of SEQ ID NO: 39, 40, 41 or 42 or an amino acid sequence having at least 70% sequence identity with SEQ ID NO: 39, 40, 41 or 42, and/or wherein the polypeptide having GABA-TA activity comprises the amino acid sequence of any one of SEQ ID NOs: 43 to 74 or an amino acid sequence having at least 70% sequence identity with any one of SEQ ID NO: 43 to 74.

24. The bacterium according to claim 1, wherein said bacterium belongs to the genus Methylobacillus or Methylobacterium.

25. The bacterium according to claim 1, wherein said bacterium is Methylobacillus flagellatus.

26. The bacterium according to claim 1, wherein the bacterium further expresses a polypeptide having pyrrolidone synthase (PYS) activity.

27. The bacterium according to claim 26, wherein the polypeptide having pyrrolidone synthase (PYS) activity comprises the amino acid sequence of SEQ ID NO: 75 or an amino acid sequence having at least 70% sequence identity with SEQ ID NO: 75.

28. A method for the production of a biochemical comprising cultivating a bacterium according to claim 1 under suitable culture conditions in a culture medium comprising a reduced one-carbon compound or a multi-carbon compound that contains no carbon-carbon bonds.

29. The method according to claim 28, wherein said biochemical is GABA or a derivative thereof.


SEQUENCE LISTING (92 sequences)

SEQ ID NO: 1 - protein, 466 aa, Escherichia coli
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 2 - protein, 457 aa, Sulfobacillus thermosulfidooxidans
MKKASEEVAINPLFARRGEESVPRYRLSEEGLLPETAYQIVHDELTLDGSARLNLATFVGTWMEPEAQKLYVEAANKNIIDKDEYPETAAIEERCLKILANLWNAPDPANSIGVSTTGSSEACMLGGLALKRRWQLSRRQQGKSTDHPNIVFSSAVQVVWEKFANYFEVEPRYVNITPEHPYLTPEGVIKAVDENTIGVVPILGVTYTGSYEPVEAIAQALDDLEKRTGLSIPIVDAASGGFVAPFLQPDLVWDFRLPRVHSINTSGHKYGLVYPGLGWVLWRDKTLLPEELIFNVSYLGGSMPTFGLNFSRPGAQVLLQYYNFLRLGRQGYYTVQKTCQNVAHFLAQEIGAMEPFELVTDGSDLPVFAWRKRPEENPLWSLEDLSFVLRERGWQVPAYPLPPTMQDVTIMRIVVRNSFSMDMAQLFLDDLNRAVAYLQGLDHPFPQALRQHKPFAH

SEQ ID NO: 3 - protein, 467 aa, Bacillus megaterium
MPQWHPHREQKNLPDEFPVNPLFSRQGEVTIPRLRISDQGMLPETAYQIIHDEIALDGNARLNLATFVTTWMEPDAKRLYGESFDKNMIDKDEYPQTAAIEERCVRILADLWNSPNPDTTMGVSTTGSSEACMLGGLALKRRWQKLRKSKGLSTDRPNIVFSSSVQVVWEKFANYWDVEPRYVNINPDHPYLDAEGVINAVDENTIGVVPILGVTYTGVYEPIAAIAKALDELQEKTGLDIPIHVDAASGGFIAPFLQPDLIWDFRLPRVKSINVSGHKYGLVYPGLGWVIWREKEDLPEDLIFRVSYLGGNMPTFALNFSRPGAQVLLQYYNFLRLGKDGYYAVQKTSQENALFLSKEIGEMEAFEILADGSDIPVLAWKLKEGYTPNWTLYDLSRQLRTYGWQVPAYPLPADMEEITIMRIVVRNGFSRDLAHLFMVNFKQAVEFLNSLDRPVLKDTKYDNGFHH

SEQ ID NO: 4 - protein, 467 aa, Listeria innocua
MLYSENDKRKHESYRIPLFGSEEESTSIPKYVLKKEPMEPRIAYQLVKDQLMDEGNARQNLATFCQTYMEKEAEILMAETLEKNAIDKSEYPQTAELENRCVNILADLWNAPKEMSYLGTSTVGSSEACMLGGLAMKFRWRNNAEKRGLDIQAKRPNLIISSGYQVCWEKFCVYWDVDMRVVPMDKEHLSLDVEKVFELVDEYTIGIVGILGITYTGKFDDIALLDEKVEAYNEANEHQLVIHIDGASGAMFTPFVNPELPWDFRLKNVVSINTSGHKYGLVYPGVGWILWKDKEYLPKELIFEVSYLGGSMPTMAINFSRSASQIIGQYYNFLRYGFEGYREIHEKTKKTALYLSKTVEKSGYFEIINDGSNLPIVCYKLKDDLDVEWTLYDLADQLLMKGWQVPAYPLPADLSDTIIQRFVCRADLGYNVAEEFAADFADALHNLEHARVLYHDKERNDSYGFTH

SEQ ID NO: 5 - protein, 462 aa, Listeria monocytogenes
MFKTNVEQNNVPVFGSFESGQDLPEKRMNKESVDPRIAYQLVKDQLIDEGSARQNLATFCQTYMEPEAEQIMAETMEKNAIDKSEYPQTAKLESSCVNMLADLWNVDESEHYMGTSTVGSSEACMLGGMAMKFRWRSAALKNGLDIHAKKPSLVISSGYQVCWEKFCVYWDIELREVPMSEEHLSINTDIIMDYVDEYTIGIVGILGITYTGKFDDIMTLNDLVEDYNNTHDNEVVIHVDGASGAMFTPFVEPGLEWDFRLPNVVSINTSGHKYGLVYPGVGWILWRDKEYLPEELVFDVSYLGGHMPTMAINFSRSASQIIGQYYNFLRFGYEGYRQIHMRTRDGALQLSQAVAETGLFEIYNDGANLPIVCYKLKDDANVAWTLYDLADRLQMKGWQVPAYPLPKEMGNTIIQRYVCRGDLGQNMVTAFKNDLSESIEELNNAHILYHDVNTSKTHGFTH

SEQ ID NO: 6 - protein, 466 aa, Shigella flexneri
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKQGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 7 - protein, 466 aa, Lactococcus lactis subsp. lactis
MLYGKENRDEAEFLEPIFGSESEQVDLPKYKLAQQSIEPRVAYQLVQDEMLDEGNARLNLATFCQTYMEPEAVKLMSQTLEKNAIDKSEYPRTTEIENRCVNMIADLWNASEKEKFMGTSTIGSSEACMLGGMAMKFSWRKRAEKLGLDINAKKPNLVISSGYQVCWEKFCIYWDIEMREVPMDKEHMSINLDKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMFLAKEIEKTGMFEIMNDGSQLPIVCYKLKEDSNRGWNLYDLADRLLMKGWQVPAYPLPKNLENEIIQRLVIRADFGMNMAFNYVQDMQEAIEALNKAHILYHEEPENKTYGFTH

SEQ ID NO: 8 - protein, 466 aa, Lactococcus lactis subsp. cremoris
MLYGKENRDEAEFLEPIFGSESEQVDLPKYKLAQQSIEPRVAYQLVQDEMLDEGNARLNLATFCQTYMEPEAVKLMSQTLEKNAIDKSEYPRTTEIENRCVNMIADLWNASEKEKFMGTSTIGSSEACMLGGMAMKFSWRKRAEKLGLDINAKKPNLVISSGYQVCWEKFCVYWDIEMREVPMDREHMSINLEKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMYLAEEIEKTGMFEIMNDGAQLPIVCYKLKENSNRGWNLYDLADRLLMKGWQVPAYPLPKNLENEIIQRLVIRADFGMNMAFNYVQDMQEAIDALNKAHILFHQEPENKTYGFTH

SEQ ID NO: 9 - protein, 464 aa, Aliivibrio fischeri
MPLHSKNAVRDDLLDDIYSSADLSLSMPKYKMPEQEHDPRHAYQVIHDELMMDGNSRQNLATFCQTWVEDEVHKLMDECIDKNMIDKDEYPQTAELESRCVHMLADLWNSPDAENTLGCSTTGSSEAAMLGGMALKWAWREKMKKLGKPTDKPNMICGPVQVCWHKFARYWDIELREIPMEGDRLIMTPEEVIKRCDENTIGVVPTLGVTFTCQYEPVKAVHEALDKLQEETGLDIPMHIDAASGGFLAPFCDPDLEWDFRLPRVKSINASGHKFGLSPLGVGWVIWRDASALHEDLIFNVNYLGGNMPTFALNFSRPGGQIVAQYYNFLRLGKEGYRKIHQACYDTAVYLSSEIEKLGMFEIIYDGKGGIPAMSWSLKEGVDPGFNLFDLSDRIRSRGWQIAAYAMPPKREDLVIMRILVRHGFSRDQADLLVADLKHCVEFFAKHPISHGSDELESSGFNHG

SEQ ID NO: 10 - protein, 468 aa, Lactobacillus brevis
MAMLYGKHTHETDETLTPIFGATAERHDLPKYKLAKHALEPREADRLVRDQLLDEGNSRLNLATFCQTYMEPEAVELMKDTLEKNAIDKSEYPRTAEIENRCVNIIANLWHAPEAESFTGTSTIGSSEACMLAGLAMKFAWRKRAKANGLDLTAHQPNIVISAGYQVCWEKFCVYWDIDMHVVPMDDDHMSLNVDHVLDYVDDYTIGIVGIMGITYTGQYDDLARLDAVVERYNRTTKFPVYIHVDAASGGFYTPFIEPELKWDFRLNNVISINASGHKYGLVYPGVGWVIWRDQQYLPKELVFKVSYLGGELPTMAINFSHSASQLIGQYYNFIRFGFDGYREIQEKTHDVARYLAKSLTKLGGFSLINDGHELPLICYELTADSDREWTLYDLSDRLLMKGWQVPTYPLPKNMTDRVIQRIVVRADFGMSMAHDFIDDLTQAIHDLDQAHIVFHSDPQPKKYGFTH

SEQ ID NO: 11 - protein, 466 aa, Artificial Sequence
  E. coli GAD mutant E89Q
MDKKQVTDLRSELLDSRFGAKSISTIAESKRFPLHEMRDDVAFQIINDELYLDGNARQNLATFCQTWDDENVHKLMDLSINKNWIDKEQYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 12 - protein, 466 aa, Artificial Sequence
  E. coli GAD mutant H465A
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKAT

SEQ ID NO: 13 - protein, 466 aa, Artificial Sequence
  E. coli GAD mutant E89Q+H465A
MDKKQVTDLRSELLDSRFGAKSISTIAESKRFPLHEMRDDVAFQIINDELYLDGNARQNLATFCQTWDDENVHKLMDLSINKNWIDKEQYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKAT

SEQ ID NO: 14 - protein, 457 aa, Artificial Sequence
  S. thermosulfidooxidans GAD mutant E84Q
MKKASEEVAINPLFARRGEESVPRYRLSEEGLLPETAYQIVHDELTLDGSARLNLATFVGTWMEPEAQKLYVEAANKNIIDKDQYPETAAIEERCLKILANLWNAPDPANSIGVSTTGSSEACMLGGLALKRRWQLSRRQQGKSTDHPNIVFSSAVQVVWEKFANYFEVEPRYVNITPEHPYLTPEGVIKAVDENTIGVVPILGVTYTGSYEPVEAIAQALDDLEKRTGLSIPIVDAASGGFVAPFLQPDLVWDFRLPRVHSINTSGHKYGLVYPGLGWVLWRDKTLLPEELIFNVSYLGGSMPTFGLNFSRPGAQVLLQYYNFLRLGRQGYYTVQKTCQNVAHFLAQEIGAMEPFELVTDGSDLPVFAWRKRPEENPLWSLEDLSFVLRERGWQVPAYPLPPTMQDVTIMRIVVRNSFSMDMAQLFLDDLNRAVAYLQGLDHPFPQALRQHKPFAH

SEQ ID NO: 15 - protein, 457 aa, Artificial Sequence
  S. thermosulfidooxidans GAD mutant H457A
MKKASEEVAINPLFARRGEESVPRYRLSEEGLLPETAYQIVHDELTLDGSARLNLATFVGTWMEPEAQKLYVEAANKNIIDKDEYPETAAIEERCLKILANLWNAPDPANSIGVSTTGSSEACMLGGLALKRRWQLSRRQQGKSTDHPNIVFSSAVQVVWEKFANYFEVEPRYVNITPEHPYLTPEGVIKAVDENTIGVVPILGVTYTGSYEPVEAIAQALDDLEKRTGLSIPIVDAASGGFVAPFLQPDLVWDFRLPRVHSINTSGHKYGLVYPGLGWVLWRDKTLLPEELIFNVSYLGGSMPTFGLNFSRPGAQVLLQYYNFLRLGRQGYYTVQKTCQNVAHFLAQEIGAMEPFELVTDGSDLPVFAWRKRPEENPLWSLEDLSFVLRERGWQVPAYPLPPTMQDVTIMRIVVRNSFSMDMAQLFLDDLNRAVAYLQGLDHPFPQALRQHKPFAA

SEQ ID NO: 16 - protein, 457 aa, Artificial Sequence
  S. thermosulfidooxidans GAD mutant E84Q+H457A
MKKASEEVAINPLFARRGEESVPRYRLSEEGLLPETAYQIVHDELTLDGSARLNLATFVGTWMEPEAQKLYVEAANKNIIDKDQYPETAAIEERCLKILANLWNAPDPANSIGVSTTGSSEACMLGGLALKRRWQLSRRQQGKSTDHPNIVFSSAVQVVWEKFANYFEVEPRYVNITPEHPYLTPEGVIKAVDENTIGVVPILGVTYTGSYEPVEAIAQALDDLEKRTGLSIPIVDAASGGFVAPFLQPDLVWDFRLPRVHSINTSGHKYGLVYPGLGWVLWRDKTLLPEELIFNVSYLGGSMPTFGLNFSRPGAQVLLQYYNFLRLGRQGYYTVQKTCQNVAHFLAQEIGAMEPFELVTDGSDLPVFAWRKRPEENPLWSLEDLSFVLRERGWQVPAYPLPPTMQDVTIMRIVVRNSFSMDMAQLFLDDLNRAVAYLQGLDHPFPQALRQHKPFAA

SEQ ID NO: 17 - protein, 467 aa, Artificial Sequence
  B. megaterium GAD mutant E93Q
MPQWHPHREQKNLPDEFPVNPLFSRQGEVTIPRLRISDQGMLPETAYQIIHDEIALDGNARLNLATFVTTWMEPDAKRLYGESFDKNMIDKDQYPQTAAIEERCVRILADLWNSPNPDTTMGVSTTGSSEACMLGGLALKRRWQKLRKSKGLSTDRPNIVFSSSVQVVWEKFANYWDVEPRYVNINPDHPYLDAEGVINAVDENTIGVVPILGVTYTGVYEPIAAIAKALDELQEKTGLDIPIHVDAASGGFIAPFLQPDLIWDFRLPRVKSINVSGHKYGLVYPGLGWVIWREKEDLPEDLIFRVSYLGGNMPTFALNFSRPGAQVLLQYYNFLRLGKDGYYAVQKTSQENALFLSKEIGEMEAFEILADGSDIPVLAWKLKEGYTPNWTLYDLSRQLRTYGWQVPAYPLPADMEEITIMRIVVRNGFSRDLAHLFMVNFKQAVEFLNSLDRPVLKDTKYDNGFHH

SEQ ID NO: 18 - protein, 467 aa, Artificial Sequence
  B. megaterium GAD mutant H467A
MPQWHPHREQKNLPDEFPVNPLFSRQGEVTIPRLRISDQGMLPETAYQIIHDEIALDGNARLNLATFVTTWMEPDAKRLYGESFDKNMIDKDEYPQTAAIEERCVRILADLWNSPNPDTTMGVSTTGSSEACMLGGLALKRRWQKLRKSKGLSTDRPNIVFSSSVQVVWEKFANYWDVEPRYVNINPDHPYLDAEGVINAVDENTIGVVPILGVTYTGVYEPIAAIAKALDELQEKTGLDIPIHVDAASGGFIAPFLQPDLIWDFRLPRVKSINVSGHKYGLVYPGLGWVIWREKEDLPEDLIFRVSYLGGNMPTFALNFSRPGAQVLLQYYNFLRLGKDGYYAVQKTSQENALFLSKEIGEMEAFEILADGSDIPVLAWKLKEGYTPNWTLYDLSRQLRTYGWQVPAYPLPADMEEITIMRIVVRNGFSRDLAHLFMVNFKQAVEFLNSLDRPVLKDTKYDNGFHA

SEQ ID NO: 19 - protein, 467 aa, Artificial Sequence
  B. megaterium GAD mutant E93Q+H467A
MPQWHPHREQKNLPDEFPVNPLFSRQGEVTIPRLRISDQGMLPETAYQIIHDEIALDGNARLNLATFVTTWMEPDAKRLYGESFDKNMIDKDQYPQTAAIEERCVRILADLWNSPNPDTTMGVSTTGSSEACMLGGLALKRRWQKLRKSKGLSTDRPNIVFSSSVQVVWEKFANYWDVEPRYVNINPDHPYLDAEGVINAVDENTIGVVPILGVTYTGVYEPIAAIAKALDELQEKTGLDIPIHVDAASGGFIAPFLQPDLIWDFRLPRVKSINVSGHKYGLVYPGLGWVIWREKEDLPEDLIFRVSYLGGNMPTFALNFSRPGAQVLLQYYNFLRLGKDGYYAVQKTSQENALFLSKEIGEMEAFEILADGSDIPVLAWKLKEGYTPNWTLYDLSRQLRTYGWQVPAYPLPADMEEITIMRIVVRNGFSRDLAHLFMVNFKQAVEFLNSLDRPVLKDTKYDNGFHA

SEQ ID NO: 20 - protein, 467 aa, Artificial Sequence
  L. innocua GAD mutant E90Q
MLYSENDKRKHESYRIPLFGSEEESTSIPKYVLKKEPMEPRIAYQLVKDQLMDEGNARQNLATFCQTYMEKEAEILMAETLEKNAIDKSQYPQTAELENRCVNILADLWNAPKEMSYLGTSTVGSSEACMLGGLAMKFRWRNNAEKRGLDIQAKRPNLIISSGYQVCWEKFCVYWDVDMRVVPMDKEHLSLDVEKVFELVDEYTIGIVGILGITYTGKFDDIALLDEKVEAYNEANEHQLVIHIDGASGAMFTPFVNPELPWDFRLKNVVSINTSGHKYGLVYPGVGWILWKDKEYLPKELIFEVSYLGGSMPTMAINFSRSASQIIGQYYNFLRYGFEGYREIHEKTKKTALYLSKTVEKSGYFEIINDGSNLPIVCYKLKDDLDVEWTLYDLADQLLMKGWQVPAYPLPADLSDTIIQRFVCRADLGYNVAEEFAADFADALHNLEHARVLYHDKERNDSYGFTH

SEQ ID NO: 21 - protein, 467 aa, Artificial Sequence
  L. innocua GAD mutant H467A
MLYSENDKRKHESYRIPLFGSEEESTSIPKYVLKKEPMEPRIAYQLVKDQLMDEGNARQNLATFCQTYMEKEAEILMAETLEKNAIDKSEYPQTAELENRCVNILADLWNAPKEMSYLGTSTVGSSEACMLGGLAMKFRWRNNAEKRGLDIQAKRPNLIISSGYQVCWEKFCVYWDVDMRVVPMDKEHLSLDVEKVFELVDEYTIGIVGILGITYTGKFDDIALLDEKVEAYNEANEHQLVIHIDGASGAMFTPFVNPELPWDFRLKNVVSINTSGHKYGLVYPGVGWILWKDKEYLPKELIFEVSYLGGSMPTMAINFSRSASQIIGQYYNFLRYGFEGYREIHEKTKKTALYLSKTVEKSGYFEIINDGSNLPIVCYKLKDDLDVEWTLYDLADQLLMKGWQVPAYPLPADLSDTIIQRFVCRADLGYNVAEEFAADFADALHNLEHARVLYHDKERNDSYGFTA

SEQ ID NO: 22 - protein, 467 aa, Artificial Sequence
  L. innocua GAD mutant E90Q+H467A
MLYSENDKRKHESYRIPLFGSEEESTSIPKYVLKKEPMEPRIAYQLVKDQLMDEGNARQNLATFCQTYMEKEAEILMAETLEKNAIDKSQYPQTAELENRCVNILADLWNAPKEMSYLGTSTVGSSEACMLGGLAMKFRWRNNAEKRGLDIQAKRPNLIISSGYQVCWEKFCVYWDVDMRVVPMDKEHLSLDVEKVFELVDEYTIGIVGILGITYTGKFDDIALLDEKVEAYNEANEHQLVIHIDGASGAMFTPFVNPELPWDFRLKNVVSINTSGHKYGLVYPGVGWILWKDKEYLPKELIFEVSYLGGSMPTMAINFSRSASQIIGQYYNFLRYGFEGYREIHEKTKKTALYLSKTVEKSGYFEIINDGSNLPIVCYKLKDDLDVEWTLYDLADQLLMKGWQVPAYPLPADLSDTIIQRFVCRADLGYNVAEEFAADFADALHNLEHARVLYHDKERNDSYGFTA

SEQ ID NO: 23 - protein, 462 aa, Artificial Sequence
  L. monocytogenes GAD mutant E85Q
MFKTNVEQNNVPVFGSFESGQDLPEKRMNKESVDPRIAYQLVKDQLIDEGSARQNLATFCQTYMEPEAEQIMAETMEKNAIDKSQYPQTAKLESSCVNMLADLWNVDESEHYMGTSTVGSSEACMLGGMAMKFRWRSAALKNGLDIHAKKPSLVISSGYQVCWEKFCVYWDIELREVPMSEEHLSINTDIIMDYVDEYTIGIVGILGITYTGKFDDIMTLNDLVEDYNNTHDNEVVIHVDGASGAMFTPFVEPGLEWDFRLPNVVSINTSGHKYGLVYPGVGWILWRDKEYLPEELVFDVSYLGGHMPTMAINFSRSASQIIGQYYNFLRFGYEGYRQIHMRTRDGALQLSQAVAETGLFEIYNDGANLPIVCYKLKDDANVAWTLYDLADRLQMKGWQVPAYPLPKEMGNTIIQRYVCRGDLGQNMVTAFKNDLSESIEELNNAHILYHDVNTSKTHGFTH

SEQ ID NO: 24 - protein, 462 aa, Artificial Sequence
  L. monocytogenes GAD mutant H462A
MFKTNVEQNNVPVFGSFESGQDLPEKRMNKESVDPRIAYQLVKDQLIDEGSARQNLATFCQTYMEPEAEQIMAETMEKNAIDKSEYPQTAKLESSCVNMLADLWNVDESEHYMGTSTVGSSEACMLGGMAMKFRWRSAALKNGLDIHAKKPSLVISSGYQVCWEKFCVYWDIELREVPMSEEHLSINTDIIMDYVDEYTIGIVGILGITYTGKFDDIMTLNDLVEDYNNTHDNEVVIHVDGASGAMFTPFVEPGLEWDFRLPNVVSINTSGHKYGLVYPGVGWILWRDKEYLPEELVFDVSYLGGHMPTMAINFSRSASQIIGQYYNFLRFGYEGYRQIHMRTRDGALQLSQAVAETGLFEIYNDGANLPIVCYKLKDDANVAWTLYDLADRLQMKGWQVPAYPLPKEMGNTIIQRYVCRGDLGQNMVTAFKNDLSESIEELNNAHILYHDVNTSKTHGFTA

SEQ ID NO: 25 - protein, 462 aa, Artificial Sequence
  L. monocytogenes GAD mutant E85Q+H462A
MFKTNVEQNNVPVFGSFESGQDLPEKRMNKESVDPRIAYQLVKDQLIDEGSARQNLATFCQTYMEPEAEQIMAETMEKNAIDKSQYPQTAKLESSCVNMLADLWNVDESEHYMGTSTVGSSEACMLGGMAMKFRWRSAALKNGLDIHAKKPSLVISSGYQVCWEKFCVYWDIELREVPMSEEHLSINTDIIMDYVDEYTIGIVGILGITYTGKFDDIMTLNDLVEDYNNTHDNEVVIHVDGASGAMFTPFVEPGLEWDFRLPNVVSINTSGHKYGLVYPGVGWILWRDKEYLPEELVFDVSYLGGHMPTMAINFSRSASQIIGQYYNFLRFGYEGYRQIHMRTRDGALQLSQAVAETGLFEIYNDGANLPIVCYKLKDDANVAWTLYDLADRLQMKGWQVPAYPLPKEMGNTIIQRYVCRGDLGQNMVTAFKNDLSESIEELNNAHILYHDVNTSKTHGFTA

SEQ ID NO: 26 - protein, 466 aa, Artificial Sequence
  S. flexneri GAD mutant E89Q
MDQKLLTDFRSELLDSRFGAKAISTIAESKRFPLHEMRDDVAFQIINDELYLDGNARQNLATFCQTWDDENVHKLMDLSINKNWIDKEQYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKQGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 27 - protein, 466 aa, Artificial Sequence
  S. flexneri GAD mutant H465A
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKQGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKAT

SEQ ID NO: 28 - protein, 466 aa, Artificial Sequence
  S. flexneri GAD mutant E89Q+H465A
MDQKLLTDFRSELLDSRFGAKAISTIAESKRFPLHEMRDDVAFQIINDELYLDGNARQNLATFCQTWDDENVHKLMDLSINKNWIDKEQYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKQGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKAT

SEQ ID NO: 29 - protein, 466 aa, Artificial Sequence
  L. lactis subsp. lactis GAD mutant E89Q
MLYGKENRDEAEFLEPIFGSESEQVDLPKYKLAQQSIEPRVAYQLVQDEMLDEGNARLNLATFCQTYMEPEAVKLMSQTLEKNAIDKSQYPRTTEIENRCVNMIADLWNASEKEKFMGTSTIGSSEACMLGGMAMKFSWRKRAEKLGLDINAKKPNLVISSGYQVCWEKFCIYWDIEMREVPMDKEHMSINLDKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMFLAKEIEKTGMFEIMNDGSQLPIVCYKLKEDSNRGWNLYDLADRLLMKGWQVPAYPLPKNLENEIIQRLVIRADFGMNMAFNYVQDMQEAIEALNKAHILYHEEPENKTYGFTH

SEQ ID NO: 30 - protein, 466 aa, Artificial Sequence
  L. lactis subsp. lactis GAD mutant H466A
MLYGKENRDEAEFLEPIFGSESEQVDLPKYKLAQQSIEPRVAYQLVQDEMLDEGNARLNLATFCQTYMEPEAVKLMSQTLEKNAIDKSEYPRTTEIENRCVNMIADLWNASEKEKFMGTSTIGSSEACMLGGMAMKFSWRKRAEKLGLDINAKKPNLVISSGYQVCWEKFCIYWDIEMREVPMDKEHMSINLDKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMFLAKEIEKTGMFEIMNDGSQLPIVCYKLKEDSNRGWNLYDLADRLLMKGWQVPAYPLPKNLENEIIQRLVIRADFGMNMAFNYVQDMQEAIEALNKAHILYHEEPENKTYGFTA

SEQ ID NO: 31 - protein, 466 aa, Artificial Sequence
  L. lactis subsp. lactis GAD mutant E89Q+H466A
MLYGKENRDEAEFLEPIFGSESEQVDLPKYKLAQQSIEPRVAYQLVQDEMLDEGNARLNLATFCQTYMEPEAVKLMSQTLEKNAIDKSQYPRTTEIENRCVNMIADLWNASEKEKFMGTSTIGSSEACMLGGMAMKFSWRKRAEKLGLDINAKKPNLVISSGYQVCWEKFCIYWDIEMREVPMDKEHMSINLDKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMFLAKEIEKTGMFEIMNDGSQLPIVCYKLKEDSNRGWNLYDLADRLLMKGWQVPAYPLPKNLENEIIQRLVIRADFGMNMAFNYVQDMQEAIEALNKAHILYHEEPENKTYGFTA

SEQ ID NO: 32 - protein, 466 aa, Artificial Sequence
  L. lactis subsp. cremoris GAD mutant E89Q
MLYGKENRDEAEFLEPIFGSESEQVDLPKYKLAQQSIEPRVAYQLVQDEMLDEGNARLNLATFCQTYMEPEAVKLMSQTLEKNAIDKSQYPRTTEIENRCVNMIADLWNASEKEKFMGTSTIGSSEACMLGGMAMKFSWRKRAEKLGLDINAKKPNLVISSGYQVCWEKFCVYWDIEMREVPMDREHMSINLEKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMYLAEEIEKTGMFEIMNDGAQLPIVCYKLKENSNRGWNLYDLADRLLMKGWQVPAYPLPKNLENEIIQRLVIRADFGMNMAFNYVQDMQEAIDALNKAHILFHQEPENKTYGFTH

SEQ ID NO: 33 - protein, 466 aa, Artificial Sequence
  L. lactis subsp. cremoris GAD mutant H466A
MLYGKENRDEAEFLEPIFGSESEQVDLPKYKLAQQSIEPRVAYQLVQDEMLDEGNARLNLATFCQTYMEPEAVKLMSQTLEKNAIDKSEYPRTTEIENRCVNMIADLWNASEKEKFMGTSTIGSSEACMLGGMAMKFSWRKRAEKLGLDINAKKPNLVISSGYQVCWEKFCVYWDIEMREVPMDREHMSINLEKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMYLAEEIEKTGMFEIMNDGAQLPIVCYKLKENSNRGWNLYDLADRLLMKGWQVPAYPLPKNLENEIIQRLVIRADFGMNMAFNYVQDMQEAIDALNKAHILFHQEPENKTYGFTA

SEQ ID NO: 34 - protein, 466 aa, Artificial Sequence
  L. lactis subsp. cremoris GAD mutant E89Q+H466A
MLYGKENRDEAEFLEPIFGSESEQVDLPKYKLAQQSIEPRVAYQLVQDEMLDEGNARLNLATFCQTYMEPEAVKLMSQTLEKNAIDKSQYPRTTEIENRCVNMIADLWNASEKEKFMGTSTIGSSEACMLGGMAMKFSWRKRAEKLGLDINAKKPNLVISSGYQVCWEKFCVYWDIEMREVPMDREHMSINLEKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMYLAEEIEKTGMFEIMNDGAQLPIVCYKLKENSNRGWNLYDLADRLLMKGWQVPAYPLPKNLENEIIQRLVIRADFGMNMAFNYVQDMQEAIDALNKAHILFHQEPENKTYGFTA

SEQ ID NO: 35 - protein, 464 aa, Artificial Sequence
  A. fischeri GAD mutant E89Q
MPLHSKNAVRDDLLDDIYSSADLSLSMPKYKMPEQEHDPRHAYQVIHDELMMDGNSRQNLATFCQTWVEDEVHKLMDECIDKNMIDKDQYPQTAELESRCVHMLADLWNSPDAENTLGCSTTGSSEAAMLGGMALKWAWREKMKKLGKPTDKPNMICGPVQVCWHKFARYWDIELREIPMEGDRLIMTPEEVIKRCDENTIGVVPTLGVTFTCQYEPVKAVHEALDKLQEETGLDIPMHIDAASGGFLAPFCDPDLEWDFRLPRVKSINASGHKFGLSPLGVGWVIWRDASALHEDLIFNVNYLGGNMPTFALNFSRPGGQIVAQYYNFLRLGKEGYRKIHQACYDTAVYLSSEIEKLGMFEIIYDGKGGIPAMSWSLKEGVDPGFNLFDLSDRIRSRGWQIAAYAMPPKREDLVIMRILVRHGFSRDQADLLVADLKHCVEFFAKHPISHGSDELESSGFNHG

SEQ ID NO: 36 - protein, 464 aa, Artificial Sequence
  A. fischeri GAD mutant H463A
MPLHSKNAVRDDLLDDIYSSADLSLSMPKYKMPEQEHDPRHAYQVIHDELMMDGNSRQNLATFCQTWVEDEVHKLMDECIDKNMIDKDEYPQTAELESRCVHMLADLWNSPDAENTLGCSTTGSSEAAMLGGMALKWAWREKMKKLGKPTDKPNMICGPVQVCWHKFARYWDIELREIPMEGDRLIMTPEEVIKRCDENTIGVVPTLGVTFTCQYEPVKAVHEALDKLQEETGLDIPMHIDAASGGFLAPFCDPDLEWDFRLPRVKSINASGHKFGLSPLGVGWVIWRDASALHEDLIFNVNYLGGNMPTFALNFSRPGGQIVAQYYNFLRLGKEGYRKIHQACYDTAVYLSSEIEKLGMFEIIYDGKGGIPAMSWSLKEGVDPGFNLFDLSDRIRSRGWQIAAYAMPPKREDLVIMRILVRHGFSRDQADLLVADLKHCVEFFAKHPISHGSDELESSGFNAG

SEQ ID NO: 37 - protein, 464 aa, Artificial Sequence
  A. fischeri GAD mutant E89Q+H463A
MPLHSKNAVRDDLLDDIYSSADLSLSMPKYKMPEQEHDPRHAYQVIHDELMMDGNSRQNLATFCQTWVEDEVHKLMDECIDKNMIDKDQYPQTAELESRCVHMLADLWNSPDAENTLGCSTTGSSEAAMLGGMALKWAWREKMKKLGKPTDKPNMICGPVQVCWHKFARYWDIELREIPMEGDRLIMTPEEVIKRCDENTIGVVPTLGVTFTCQYEPVKAVHEALDKLQEETGLDIPMHIDAASGGFLAPFCDPDLEWDFRLPRVKSINASGHKFGLSPLGVGWVIWRDASALHEDLIFNVNYLGGNMPTFALNFSRPGGQIVAQYYNFLRLGKEGYRKIHQACYDTAVYLSSEIEKLGMFEIIYDGKGGIPAMSWSLKEGVDPGFNLFDLSDRIRSRGWQIAAYAMPPKREDLVIMRILVRHGFSRDQADLLVADLKHCVEFFAKHPISHGSDELESSGFNAG

SEQ ID NO: 38 - protein, 468 aa, Artificial Sequence
  L. brevis GAD 4AA mutant
MAMLYGKHTHETDETLIPIFGATAERHDLPKYKLAKHALEPREADRLVRDQLLDEGNSRLNLATFCQTYMEPEAVELMKDTLEKNAIDKSEYPRTAEIENRCVNIIANLWHAPEAESFTGTSTIGSSEACMLAGLAMKFAWRKRAKANGLDLTAHQPNIVISAGYQVCWEKFCVYWDIDMHVVPMDDDHMSLNVDHVLDYVDDYTIGIVGIMGITYTGQYDDLARLDAVVERYNRTTKFPVYIHVDAASGGFYTPFIEPELKWDFRLNNVISINASGHKYGLVYPGVGWVIWRGQQYLPKELVFKVSYLGGSLPTMAINFSHSASQLIGQYYNFIRFGFDGYREIHEKTHDVARYLAKSLTKLGGFSLINDGHELPLICYELTADSDREWTLYDLSDRLLMKGWQVPTYPLPKNMTDRVIQRIVVRADFGMSMAHDFIDDLTQAIHDLDQAHIVFHSDPQPKKYGFTH

SEQ ID NO: 39 - protein, 614 aa, Euglena gracilis
MGIALDGPDRDRMFNAYLHSSRPSFGPATDVFVQCLEAEKIDWIFGIPGEENLDLLHSLAKAHRREGGETMKLVVTRHEQAAGFMAATVGRLTGHPAACLSTLGPGATNFTTALAYSKLGGFPLICITGQKPIRHSKQGAFQILDICGHFRDVVKSTKSIEDPDLIPSTIRNAVLQAREEKPGPVHIELAEDIAALEVSKEGGWVFPAPTRTPIARGRRPLVEPKCIEHAVTLIHAAKRPLICIAAGANRKNTHHAMEVLMRKTGLMVVCTQMGKGVVYEDNLGYIGCTALSDKDLVHVAINTADLIVNVGHDISEKPPFIMNHNKPPLVIHANFTRPLVDPVYFPHLVLVGDINDCLWQLATRIRPQPHWDFNPFQMVRREIERTVWNSPYAQSDAFPLTVQRLVSDVRKAMPVDGIVSLDNGMYKIWFARQYKTMMGNTLLLDNALATMGAGLPSCIAAKLVYPERVCIAVCGDGGFMMNSQEIETALRLNLHIVVIVLNNNSYGMIAWKATAMGMDDFGLNYGNPDFAQYARAYGAIGHNVKSTAEFLPTLEKAIKEHGVHIIDLPISYETSDKALFEDLPKEVEELKKAVAKAISEEKKFDWDAVTAAQS

SEQ ID NO: 40 - protein, 545 aa, Synechococcus sp.
MNTAELLIRCLENEGVEYIFGLPGEENLHILEALKESPIRFITVRHEQGAAFMADVYGRLTGKAGVCLSTLGPGATNLMTGVADANLDGAPLIAITGQVGTDRMHIESHQYLDLVAMFAPVTKWNKQIVRPNTTPEVVRRAFKIAQQEKPGAVHIDLPENIAAMPVEGQPLQRDGREKIYASSRSLNRAAEAIAHAKSPLILVGNGIIRADAAEALTDFATQLNIPVVNTFMGKGAIPYTHPLSLWTVGLQQRDFVTCAFEQSDLVIAVGYDLIEYSPKRWNPEGTTPIIHIGEVAAEIDSSYIPLTEVVGDIGDALNEIRKRTDREGKTAPKFLNVRAEIREDYERHGTDASFPVKPQKIIYDLRQVMAPEDIVISDVGAHKMWMARHYHCDRPNTCLISNGFAAMGIAIPGAVAAKLVYPEKNVVAVTGDGGFMMNCQELETALRIGANFVTLIFNDGGYGLIGWKQINQFGAPAFVEFGNPDFVQFAESMGLKGYRITAAADLVPTLKEALAQDVPAVIDCPVDYSENVKFSQKSGDLICRM

SEQ ID NO: 41 - protein, 1231 aa, M. tuberculosis
MANISSPFGQNEWLVEEMYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYVYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 42 - protein, 580 aa, Bacillus subtilis
MTVNPITHYIGSFIDEFALSGITDAVVCPGSRSTPLAVLCAAHPDISVHVQIDERSAGFFALGLAKAKQRPVLLICTSGTAAANFYPAVVEAHYSRVPIIVLTADRPHELREVGAPQAINQHFLFGNFVKFFTDSALPEESPQMLRYIRTLASRAAGEAQKRPMGPVHVNVPLREPLMPDLSDEPFGRMRTGRHVSVKTGTQSVDRESLSDVAEMLAEAEKGMIVCGELHSDADKENIIALSKALQYPILADPLSNLRNGVHDKSTVIDAYDSFLKDDELKRKLRPDVVIRFGPMPVSKPVFLWLKDDPTIQQIVIDEDGGWRDPTQASAHMIHCNASVFAEEIMAGLTAATRSSEWLEKWQFVNGRFREHLQTISSEDVSFEGNLYRILQHLVPENSSLFVGNSMPIRDVDTFFEKQDRPFRIYSNRGANGIDGVVSSAMGVCEGTKAPVTLVIGDLSFYHDLNGLLAAKKLGIPLTVILVNNDGGGIFSFLPQASEKTHFEDLFGTPTGLDFKHAAALYGGTYSCPASWDEFKTAYAPQADKPGLHLIEIKTDRQSRVQLHRDMLNEAVREVKKQWEL

SEQ ID NO: 43 - protein, 426 aa, Escherichia coli
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 44 - protein, 504 aa, Arabidopsis thaliana
MVVINSLRRLARTTQVHLHSKYATCMSGNSTSRRIFTTEAAPEKKNTVGSKGHDMLAPFTAGWQSADLDPLVIAKSEGSYVYDDTGKKYLDSLAGLWCTALGGNEPRLVSAAVEQLNTLPFYHSFWNRTTKPSLDLAKVLLEMFTANKMAKAFFTSGGSDANDTQVKLVWYYNNALGRPEKKKFIARKKSYHGSTLISASLSGLPPLHQNFDLPAPFVLHTDCPHYWRFHLPGETEEEFSTRLAKNLEDLIIKEGPETIGAFIAEPVMGAGGVIPPPATYFEKVQAVVKKYDILFIADEVICAFGRLGTMFGCDKYNIKPDLVTLAKALSSAYMPIGAILMSQEVADVINSHSSKLGVFSHGFTYSGHPVSCAVAIEALKIYKERNIPEYVAKVAPRFQDGVKAFASGSPIIGETRGTGLILGTEFVDNKSPNEPFPPEWGVGAFFGAECQKHGMLVRVAGDGILMSPPLIISPEEIDELISIYGKALKATEEKVKELKAQHKK

SEQ ID NO: 45 - protein, 515 aa, Solanum lycopersicum
MAKISRLFGSTVKAAITAQAGFHGKRIPAVSSLQEHIVKSTPARYNSTQACLENDISGTDNKGFKGHDMLAPFTAGWQSTDVDPLIIEKSEGSHVYDMQGRKYIDTLAGLWCTALGGNEPRLVDAATKQLNTLPFYHSFWNRTTKPSLDLAKELLDMFTAKKMAKAFFTNSGSEANDTQVKLVWYYNNALGRPNKKKFIARAKAYHGSTLISASLTGLPALHQNFDLPAPFVLHTDCPHYWRYHLPGETEEEFSTRLAKNLEDLILKEGPETIAAFIAEPVMGAGGVIPPPATYFDKIQAVVKKYDILFIADEVICAFGRLGTMFGSDMYNIKPDLVTLAKALSSAYMPIGAVLVSPEVSDVIHSQSNKLGSFSHGFTYSGHPVACAVALEAIKIYKERNMVERVNRISPKFQEGLKAFSDSPIIGEIRGLGLILATEFANNKSPNDHFPPEWGVGAYFGAQCQKNGMLVRVAGDTIMMSPPFVVTPEELDELIRIYGKALRETEKRVEELKSQK

SEQ ID NO: 46 - protein, 520 aa, Solanum lycopersicum
MAKITSLIGSGIVAATNQVGPHVKHIPAVGNLQKQIVSDQIQVRWSSTETSLKNDISATDVRGYKGHDMLAPFTAGWHSTDLEPLVIQKSEGSYVYDVNGKKYLDALAGLWCTSLGGNEPRLVAAATKQLNELAFYHSFWNRSTKPSLDLAKELLDLFTANKMAKAFFTNSGSEANDTQVKLVWYYNNALGRPDKKKFIARTKSYHGSTLISASLSGLPALHQQFDLPAPFVLHTDCPHFWRFHQPGETEEEFSTRLANNLENLILKEGPETIAAFIAEPVMGAGGVIPPPATYFEKVQAILKKYDILFIADEVICGFGRLGTMFGCEKYNIKPDLVSVAKALSSGYMPIGAVLVSPEVSDVIYSQSNKLGTFSHGFTYSGHPVSCAVALETLKIYKERNIIEQVNRISPKFQEGLKAFSDSPIIGEIRGTGLLHGTEFTDNKSPNDPFPPEWGIGAYFGARCEKHGVLVRVAGDNIMMSPPYILSLEEIDELIIKYGKALKDTENRVEELKSQKKIKSS

SEQ ID NO: 47 - protein, 458 aa, Solanum lycopersicum
MAKTNGFMGHDMLAPFTAAWMIDMGPLVIDKAEGSYVYGVNGKKYLDSLSGLWCTVLGGSEPRLIEAASKQLNKSAFYHSFWNRTTKPSLDLAKELINMFTANKMGKVFFTSSGSEANDTQVKLVWYYNNAIGRPNKKKIISRKNAYHGSTYMTAGLSGLPSLHLKFDLPPPYILHTDCPHYWNYHLPGETEEEYSTRLANNLENLILKEGPETVAAFIAEPVMGGAGVIIPPATYFEKIQAVLKKYDILFIADEVICGFGRLGTMFGCDKYNIKPDLVSIAKALSGGYIPIGAVLVSEEISKVIMSQSNQLGVFCHGFTYSGHPVACAVALEALKIYKEKNITEVVNKLSPKLQEGLKAFIDSPIIGEIRGTGLVLSTEFVDNKSPNDPFPPEWGVGTYFGSQCQKHGMLVSFSGDHVNMAPPFTLSLEELDEMISIYGKALKDTEKRVEELKSQKK

SEQ ID NO: 48 - protein, 448 aa, Pseudomonas aeruginosa
MNQPLNVAPPVSSELNLRAHWMPFSANRNFQKDPRIIVAAEGSWLTDDKGRKVYDSLSGLWTCGAGHSRKEIQEAVARQLGTLDYSPGFQYGHPLSFQLAEKIAGLLPGELNHVFFTGSGSECADTSIKMARAYWRLKGQPQKTKLIGRARGYHGVNVAGTSLGGIGGNRKMFGQLMDVDHLPHTLQPGMAFTRGMAQTGGVELANELLKLIELHDASNIAAVIVEPMSGSAGVLVPPVGYLQRLREICDQHNILLIFDEVITAFGRLGTYSGAEYFGVTPDLMNVAKQVTNGAVPMGAVIASSEIYDTFMNQALPEHAVEFSHGYTYSAHPVACAAGLAALDILARDNLVQQSAELAPHFEKGLHGLQGAKNVIDIRNCGLAGAIQIAPRDGDPTVRPFEAGMKLWQQGFYVRFGGDTLQFGPTFNARPEELDRLFDAVGEALNGIA

SEQ ID NO: 49 - protein, 483 aa, Oryza sativa subsp. japonica
MTTPHGLLQYSSGAFSDQVPADDSAEEHGVKDHAMLAPFTAAWQTAISPPLVIERSEGCYVYDVNGTKYLDALAGLLSTALGGSEPRLVKAATEQLNKLPFYHSFWNHTTRPSLDLAKELISMFTAREMGKVFFTNSGSEANDSQVKIVWYYNNALGRPKKKNIISRTQSYHGTTFISASLSGLPTLHQDFDLPGRFVLHTDCPHYWRFHLPGETEEEFATRLADNLENLILKQGPETIAAFIAEPVIGAGGVILPPKTYFEKIQAVVKKYDILFIVDEVITGFGRLGTMFGSDLYNIKPDLVSLAKALSSAYAPIGAILVSPEISDVIHSHSNKLGTFAHGFTYSGHPVSCAVALEALKIYRERDIPGHVTHVAQRFQEGIKAFAAGSPIVGETRGVGLLIATEFTDNKSPYELFPFEWGVGEIFGQECKKRGMMVKVLGNLIAMSPPLIITREEIDKLVSIYGEALKATEERVAELKSKKN

SEQ ID NO: 50 - protein, 516 aa, Oryza sativa subsp. japonica
MVIARGLLRSNASSSSSQAINLLKYVTSTGSLQGHTQNLCDASTRHFSSVPSPQYNSTEENGFKGHGMLAPFTAGWQSTDVHPLVIERSEGSYVYDIDGKKYLDSLAGLWCTALGGSEPRLVKAATEQLHKLPFYHSFWNRTTKPSLDLAKELLSMFTAREMGKVFFTNSGSEANDSQVKLVWYYNNALGRPDKKKFIARSKSYHGSTLISASLSGLPALHQKFDLPAPFVLHTDCPHYWRFHLPGETEEEFATRLANNLEELILKEGPETIAAFIAEPVMGAGGVIPPPKTYFEKVQAIVKKYDILFIADEVITAFGRLGTMFGSDMYNIKPDLVSMAKALSSAYVPIGAIMVSPEISDVIHSQSNKLGSFAHGFTYSGHPVACAVAIEALKIYQERNIPDHVKQISPRFQEGVKAFAGSPIVGEIRGVGLILGTEFADNKSPNDPFPAEWGVGAIFGAECQKRGMLVRVAGDNIMMSPPLIMTPDEVEELVSIYGDALKATEERVAELKSKKNN

SEQ ID NO: 51 - protein, 497 aa, Oryza sativa subsp. japonica
MNLIKHAAFAASFQGETDCTSHASARKFSTSGSSPLLDSTEGNGFKGHSMLAPFTAGWHSTDLEPLIIERSEGSYVYDSKGNKYLDTLAGLWCTALGGSEPRLVKAATDQLNKLPFYHSFWNSTAKPPLDLAEELISMFTAKEMGKVFFTNSGSEANDSQVKLVWYYNNALGRPNKKKIIAQSQAYHGSTLISASLSGLPAMHLKFDLPAPFVLHTDCPHYWRFGLPGEAEEEFATRLADNLENLILKEGPETVAAFIAEPVIGAGGVIPPPKTYFEKIQAVLQKYDVLFIADEVITGFGRLGTMFGSDLYNIKPDLVSLAKALSSAYVPIGATLVSPEISDVVHSQSNKIGFFAHGFTYSGHPVSCAVALEALKIYRERNIPAHVKQISPRFQEGIKAFAGSSIIGETRGVGLLLATEFANNKSPNDPFPVEWGVAQIFGAECKKRGMLVKVVGDEIAMSPPLIMSQREVDGLVSIYGEALKATEERVAELRSKKK

SEQ ID NO: 52 - protein, 510 aa, Oryza sativa subsp. japonica
MICRSLLLLRSNAASKASNIVKHVAATGCLPKYSSEAPARYFSSEPSLQVDSTEENGFKGHGMLAPFTAGWQSTDLHPLVIDRSEGSYVYDINGKKYIDALAGLWSTALGGNEPRLIKAATDQLNKLPFYHSFWNRTTKPSLDLANEILSMFTAREMGKIFFTNSGSEANDSQVKLVWYYNNALGRPNKKKFIARSKSYHGSTLVSASLSGLPALHQKFDLPAPFVLHTDCPHYWRFHLPDETEEEFATRLATNLENLILKEGPETIAAFIAEPVMGAGGVIPPPKTYFEKIQAVLKKYDILLIADEVITAFGRLGTMFGCDMYDIKPDLVSIAKALSSAYMPIGAILVSPEITDVIYSQSNKLGSFAHGFTYSGHPVSCAVAIEALKIYKERNIIEHVQKIAPRFQEGIKAFSGSPIVGEIRGLGLILGTEFVDNKSPNDPFPAEWGVGSLFGAECEKRGMLIRVAGDNIMLSPPLIMTPDEVEEIICKYGDALKATEERIAELKAKRG

SEQ ID NO: 53 - protein, 497 aa, Oryza sativa subsp. indica; identical to SEQ ID NO: 51

SEQ ID NO: 54 - protein, 516 aa, Oryza sativa subsp. indica
MVIARGLLRSNASSSSSQAINLLKYVTSTGSLQGHTQNLCDASTRHFSSVPSPQSNSTEENGFKGHGMLAPFTAGWQSTDVHPLVIERSEGSYVYDIDGKKYLDSLAGLWCTALGGSEPRLAKAATEQLHKLPFYHSFWNRTTKPSLDLAKELLSMFTAREMGKVFFTNSGSEANDSQVKLVWYYNNALGRPDKKKFIARSKSYHGSTLISASLSGLPALHQKFDLPAPFVLHTDCPHYWRFHLPGETEEEFATRLANNLEELILKEGPETIAAFIAEPVMGAGGVIPPPKTYFEKVQAIVKKYDILFIADEVITAFGRLGTMFGSDMYNIKPDLVSMAKALSSAYVPIGAIMVSPEISDVIHSQSNKLGSFAHGFTYSGHPVACAVAIEALKIYQERNIPDHVKQISPRFQEGVKAFAGSPIVGEIRGVGLILGTEFADNKSPNDPFPAEWGVGAIFGAECQKRGMLVRVAGDNIMMSPPLIMTPDEVEELVSIYGDALKATEERVAELKSKKNN

SEQ ID NO: 55 - protein, 510 aa, Oryza sativa subsp. indica
MICRSLLLLRSNAASKASSIVKHVAATGCLPEYSSEAPARYFSSESSLQVDSTEENGFKGHGMLAPFTAGWQSTDLHPLVIDRSEGSYVYDINGKKYIDALAGLWSTALGGNEPRLIKAATDQLNKLPFYHSFWNRTTKPSLDLANEILSMFTAREMGKIFFTNSGSEANDSQVKLVWYYNNALGRPNKKKFIARSKSYHGSTLVSASLSGLPALHQKFDLPAPFVLHTDCPHYWRFHLPDETEEEFATRLATNLENLILKEGPETIAAFIAEPVMGAGGVIPPPKTYFEKIQAVLKKYDILLIADEVITAFGRLGTMFGCDMYDIKPDLVSIAKALSSAYMPIGAILVSPEITDVIYSQSNKLGSFAHGFTYSGHPVSCAVAIEALKIYKERNIIEHVQKIAPRFQEGIKAFSGSPIVGEIRGLGLILGTEFVDNKSPNDPFPAEWGVGSLFGAECEKRGMLIRVAGDNIMLSPPLIMTPDEVEEIISKYGDALKATEERIAELKAKRG

SEQ ID NO: 56 - protein, 426 aa, Escherichia coli; identical to SEQ ID NO: 43

SEQ ID NO: 57 - protein, 500 aa, Homo sapiens
MASMLLAQRLACSFQHSYRLLVPGSRHISQAAAKVDVEFDYDGPLMKTEVPGPRSQELMKQLNIIQNAEAVHFFCNYEESRGNYLVDVDGNRMLDLYSQISSVPIGYSHPALLKLIQQPQNASMFVNRPALGILPPENFVEKLRQSLLSVAPKGMSQLITMACGSCSNENALKTIFMWYRSKERGQRGFSQEELETCMINQAPGCPDYSILSFMGAFHGRTMGCLATTHSKAIHKIDIPSFDWPIAPFPRLKYPLEEFVKENQQEEARCLEEVEDLIVKYRKKKKTVAGIIVEPIQSEGGDNHASDDFFRKLRDIARKHGCAFLVDEVQTGGGCTGKFWAHEHWGLDDPADVMTFSKKMMTGGFFHKEEFRPNAPYRIFNTWLGDPSKNLLLAEVINIIKREDLLNNAAHAGKALLTGLLDLQARYPQFISRVRGRGTFCSFDTPDDSIRNKLILIARNKGVVLGGCGDKSIRFRPTLVFRDHHAHLFLNIFSDILADFK

SEQ ID NO: 58 - protein, 500 aa, Rattus norvegicus
MAFLLTTRRLVCSSQKNLHLFTPGSRYISQAAAKVDFEFDYDGPLMKTEVPGPRSQELMKQLNTIQNAEAVHFFCNYEESRGNYLVDVDGNRMLDLYSQISSVPIGYNHPALAKLVQQPQNASTFINRPALGILPPENFVDKLRESLMSVAPKGMCQLITMACGSCSNENAFKTIFMWYRSKERGQRGFSKEELETCMVNQSPGCPDYSILSFMGAFHGRTMGCLATTHSKAIHKIDIPSFDWPIAPFPRLKYPLEEFVTDNQQEEARCLEEVEDLIVKYRKKKRTVAGIIVEPIQSEGGDNHASDDFFRKLRDIARKHGCAFLVDEVQTGGGCTGKFWAHEHWGLDDPADVMSFSKKMMTGGFFHKEEFRPSAPYRIFNTWLGDPSKNLLLAEVINIIKREDLLNNVAHAGKTLLTGLLDLQAQYPQFVSRVRGRGTFCSFDTPDEAIRNKLILIARNKGVVLGGCGDKSIRFRPTLVFRDHHAHLFLNIFSGILADFK

SEQ ID NO: 59 - protein, 500 aa, Sus scrofa
MASVLLTRRLACSFRHNHRLLVPGWRHISQAAAKVDVEFDYDGPLMKTEVPGPRSRELMKQLNIIQNAEAVHFFCNYEESRGNYLVDVDGNRMLDLYSQISSIPIGYSHPALVKLVQQPQNVSTFINRPALGILPPENFVEKLRESLLSVAPKGMSQLITMACGSCSNENAFKTIFMWYRSKERGQSAFSKEELETCMINQAPGCPDYSILSFMGAFHGRTMGCLATTHSKAIHKIDIPSFDWPIAPFPRLKYPLEEFVKENQQEEARCLEEVEDLIVKYRKKKKTVAGIIVEPIQSEGGDNHASDDFFRKLRDISRKHGCAFLVDEVQTGGGSTGKFWAHEHWGLDDPADVMTFSKKMMTGGFFHKEEFRPNAPYRIFNTWLGDPSKNLLLAEVINIIKREDLLSNAAHAGKVLLTGLLDLQARYPQFISRVRGRGTFCSFDTPDESIRNKLISIARNKGVMLGGCGDKSIRFRPTLVFRDHHAHLFLNIFSDILADFK

SEQ ID NO: 60 - protein, 421 aa, Escherichia coli
MSNNEFHQRRLSATPRGVGVMCNFFAQSAENATLKDVEGNEYIDFAAGIAVLNTGHRHPDLVAAVEQQLQQFTHTAYQIVPYESYVTLAEKINALAPVSGQAKTAFFTTGAEAVENAVKIARAHTGRPGVIAFSGGFHGRTYMTMALTGKVAPYKIGFGPFPGSVYHVPYPSDLHGISTQDSLDAIERLFKSDIEAKQVAAIIFEPVQGEGGFNVAPKELVAAIRRLCDEHGIVMIADEVQSGFARTGKLFAMDHYADKPDLMTMAKSLAGGMPLSGVVGNANIMDAPAPGGLGGTYAGNPLAVAAAHAVLNIIDKESLCERANQLGQRLKNTLIDAKESVPAIAAVRGLGSMIAVEFNDPQTGEPSAAIAQKIQQRALAQGLLLLTCGAYGNVIRFLYPLTIPDAQFDAAMKILQDALSD

SEQ ID NO: 61 - protein, 500 aa, Mus musculus
MAFLLITRRLACSSQKNLHLFIPGSRYISQAAAKVDIEFDYDGPLMKTEVPGPRSKELMKQLNTIQNAEAVHFFCNYEESRGNYLVDVDGNRMLDLYSQISSVPIGYNHPALAKLVQQPQNASTFINRPALGILPPENFVDKLQESLMSVAPRGMSQLITMACGSCSNENAFKTIFMWYRSKERGQRGFSKEELETCMVNQSPGCPDYSILSFMGAFHGRTMGCLATTHSKAIHKIDIPSFDWPIAPFPRLKYPLEEFTTDNQQEEARCLEEVEDLIVKYRKKKRTVAGIIVEPIQSEGGDNHASDDFFRKLRDIARKHGCAFLVDEVQTGGGCTGKFWAHEHWGLDDPADVMTFSKKMMTGGFFHKEEFRPSAPYRIFNTWLGDPSKNLLLAEVINIIKREDLLNNVARVGKTLLTGLLDLQAQYPQFISRVRGRGTFCSFDTPDEAIRNKLILIARNKGVVLGGCGDKSIRFRPTLVFRDHHAHLFLSIFSGILADFK

SEQ ID NO: 62 - protein, 500 aa, Bos taurus
MASMLVAQRLACSFQHSYRLLVPGSRHISQAAAKVDVEFDYDGRLMKTEVPGLRCQELMKQLNIIQNAEAVHFFCNYEESRGNYLVDVDGNRMLDLYSQISSVPIGYSHPGLLKLIQQPQNASMFVNRPALGILLPENFVEKLRQSLLSVAPKGMSQLITMACGSCSNENGLKTIFMWYRSKERGQRGFPQEELETCMINQAPWCPDYSILSFMGAFHGRTMGCLATTHSKAIHKIDIPSFDWPIAPFPRLKYPLEEFVKENQQEEARCLEEVEDLIVKYRKKKKTVAGIIVEPIQSEGGDNHASDDFFRKLRDIPRKQCCAFLVDVVQTGGGCTGKFWAHEHWARDDPEDVMTSSKKMMTGGFFHKEEFRPNAPYRIFNTWLGDPSKNLLLAEVINIIKREDLLNNAAHAGKALLTGLLDLQARYPQFISRVRGRGTFCSFDTPDDSIRNKLILIARNKGVVLGGCGDKSIRFRPTLVFRDHHAHLFLNIFSDILADFK

SEQ ID NO: 63 - protein, 471 aa, Saccharomyces cerevisiae
MSICEQYYPEEPTKPTVKTESIPGPESQKQLKELGEVFDTRPAYFLADYEKSLGNYITDVDGNTYLDLYAQISSIALGYNNPALIKAAQSPEMIRALVDRPALGNFPSKDLDKILKQILKSAPKGQDHVWSGLSGADANELAFKAAFIYYRAKQRGYDADFSEKENLSVMDNDAPGAPHLAVLSFKRAFHGRLFASGSTTCSKPIHKLDFPAFHWPHAEYPSYQYPLDENSDANRKEDDHCLAIVEELIKTWSIPVAALIIEPIQSEGGDNHASKYFLQKLRDITLKYNVVYIIDEVQTGVGATGKLWCHEYADIQPPVDLVTFSKKFQSAGYFFHDPKFIPNKPYRQFNTWCGEPARMIIAGAIGQEISDKKLTEQCSRVGDYLFKKLEGLQKKYPENFQNLRGKGRGTFIAWDLPTGEKRDLLLKKLKLNGCNVGGCAVHAVRLRPSLTFEEKHADIFIEALAKSVNEL

SEQ ID NO: 64 - protein, 474 aa, Schizosaccharomyces pombe
MSSTATVTESTHFFPNEPQGPSIKTETIPGPKGKAAAEEMSKYHDISAVKFPVDYEKSIGNYLVDLDGNVLLDVYSQIATIPIGYNNPTLLKAAKSDEVATILMNRPALGNYPPKEWARVAYEGAIKYAPKGQKYVYFQMSGSDANEIAYKLAMLHHFNNKPRPTGDYTAEENESCLNNAAPGSPEVAVLSFRHSFHGRLFGSLSTTRSKPVHKLGMPAFPWPQADFPALKYPLEEHVEENAKEEQRCIDQVEQILTNHHCPVVACIIEPIQSEGGDNHASPDFFHKLQATLKKHDVKFIVDEVQTGVGSTGTLWAHEQWNLPYPPDMVTFSKKFQAAGIFYHDLALRPHAYQHFNTWMGDPFRAVQSRYILQEIQDKDLLNNVKSVGDFLYAGLEELARKHPGKINNLRGKGKGTFIAWDCESPAARDKFCADMRINGVNIGGCGVAAIRLRPMLVFQKHHAQILLKKIDELI

SEQ ID NO: 65 - protein, 483 aa, Caenorhabditis elegans
MLPRLAKSSLIQQVRGVSAIANAEPSGPSISTSIPGPKSKALKQEMDKVHQTTSVRFHVDYEKSFGNYVVDADGNALLDVYTQISSLPLGYNHPDLVKVASQPHLITSLVSRPALGSFPRTDFADGISHALTSIAPKGLKAVQTMLCGTSANENAIKTAFIWYQAQRRGGLGPDALHLESCMNQQKPGTPNLSVMGFEGAFHGRSLCMLSVTRSKPIHKVDIPAFDWPIAKFPRYKYPLDQNVAYNKKQDQECLADVEAKISEWKRRDNDVAAIIVEPIQAEGGDHYGSPAFFQGLRDITSKHGIVFIVDEVQTGGGATGDIWAHDHWNLSSPPDMVTFSKKLLTGGYFYGEHLRVKEAYRIYNTWMGDPTKLLLLEKAVEVIKRDGLIEQSREVGAEFQKRLGELQASSGGKLDQARGRGTFAAVDFPSGSLRDKFVDLAISNGLHCGGCGDRSLRFRPSLVYTKKHLDLTFDLLDKTLKGL

SEQ ID NO: 66 - protein, 498 aa, Emericella nidulans
MASAFRSSLKLRASARLPAVRTITTTPRLRAAEKPYFPNEPTAPKLATAIPGPKNKAASEQLNEVFDVRSLNMLADYTKSVGNYIADLDGNMLLDVYAQIASIPVGYNNPHLLKVAASPEMATSLINRPALGNFPSADWAHILKTGILKVAPKGLDQVFTAMAGSDANETAYKAAFMYYRQQQRGGPEKEFTEEEIQSSMLNQTPGSPQLSIMSFKAGFHGRLFGSLSTTRSKPIHKLDIPAFDWPQAPFPSLKYPLEEHAKENAEEEQRCLQEAERLIKEWHNPVAAIIVEPIQSEGGDNHASPAFFRGLREITKRNNVLFIVDEVQTGVGATGKFWAHDHWNLETPPDMVTFSKKAQTAGYYFGNPALRPNKPYRQFNTWMGDPSRALIFRGIIEEIERLFLVENTAATGDYLYSGLERLAKQYPEHLQNLRGKGQGTFIAWDTPKRDEFLVKGKGVGINIGGSGQNAVRLRPMLIFQKHHADILLESIEKIIKQL

SEQ ID NO: 67 - protein, 509 aa, Ustilago maydis
MISSKATSAARACARTSRVMQQQQRRLLATVVSSSSLFPGEPSSPHVVTSQIPGPKSKELSDRIGTFQENRTHGFVVDYAKSQGNWIADADGNVLLDMFAQIASIAIGYNNPDLLALAKTDEFITATMNRAALGSFPPTNWQELVETSFGTVKPDGLNNIFTAMCGSCANENAFKASFMAYRARERGEKAEFTPEEMSSCMKNQSPGSPDLSILSFTSAFHGRLFGSLSATRSKAIHKLDIPSFNWPVVEWPDVKYPFAQNSRENAEAEKVALAAVEEAIVSSKKTGSSYGPVAALIVEPIQSEGGDNHASPAFFQGLRDVTKKHGVFMIVDEVQTGVGATGAFWAHSKWNLTSPPDFVTFSKKMQAAGFYHNIETRPSLPYRNYNTWMGDPARTLQARQIIRTIQDHNLIQKTDKVGNYIYEKLFDLIENGAGRGKIEKLRGENAGTFLAFDGRTAKVRDQLIMEMRKLGVHMGGCGDKALRLRPMLVFEQKHADIFLDKLETALGQL

SEQ ID NO: 68 - protein, 472 aa, Kluyveromyces lactis
MSVAAKYYPNEPTEPKVVTSEIPGPESKAKVASLGEVFDSRPAYFVADYAKSSGNYIVDVDGNKFLDVYAQISSIALGYNNPALIEAAKSDKMIRALVDRPALGNFPGADLEDILKQLLKFAPKGQNKIWSGLSGADANELAFKAAFMYYRQLQRGGHGIDFSEEENSSVMENTSPGSPQLAVLSFKKAFHGRLFASGSSTCSKPIHKLDFPAFNWPHGEYPVYKYPLSENEEENKKEDDRCLAIVEDLIKSWPTPVAALIIEPIQSEGGDNHASKYFLQSLRDLTSKYNVVYIIDEVQTGVGATGKFWCHEWADIQPPVDLVTFSKKFQSAGYWFHDDRFIPNKAYRQFNTWCGDPARMIIAGAIGQEIVDNNLVDQCARVGDYLFEKLEKLQAKYPTRLINLRGKNRGTFIAFDLETSAERDQLLKLLKSNGCNVGGCAEKSVRLRPSLTFEEKHADIFVDALEKSIGQL

SEQ ID NO: 69 - protein, 436 aa, Bacillus subtilis
MSQTTASITTAQWQQKRDQFVSKGVSNGNRSLAVKGEGAELYDLDGRRFIDFAGAIGTLNVGHSHPKVVEAVKRQAEELIHPGFNVMMYPTYIELAEKLCGIAPGSHEKKAIFLNSGAEAVENAVKIARKYTKRQGVVSFTRGFHGRTNMTMSMTSKVKPYKFGFGPFAPEVYQAPFPYYYQKPAGMSDESYDDMVIQAFNDFFIASVAPETVACVVMEPVQGEGGFIIPSKRFVQHVASFCKEHGIVFVADEIQTGFARTGTYFAIEHFDVVPDLITVSKSLAAGLPLSGVIGRAEMLDAAAPGELGGTYAGSPLGCAAALAVLDIIEEEGLNERSEEIGKIIEDKAYEWKQEFPFIGDIRRLGAMAAIEIVKDPDTREPDKTKAAAIAAYANQNGLLLLTAGINGNIIRFLTPLVISDSLLNEGLSILEAGLRA

SEQ ID NO: 70 - protein, 495 aa, Dictyostelium discoideum
MSSSRLIKCLSSNNYIVRSFSKSSIPTTPTPDFPGEYKEPIVKTQIPGPQSKALIERLNKLQDPRAAHFFADYANSRGNYISDVDGNILLDLYCQIASIPIGYNNPELIKAAKSDRWVSAIINRPSLGVLPPKDWPALIENSFMQVSPKGLNQVFTAMCGSCANECAYKAVFMHYQHVKRGGKPFTPEELSSCMKNQEPGSPSLSILSFKKGFHGRTFGTLSTTRSKAIHKLDIPAFDWPAATFPDLKYPLAEHAKENREIEDRCLQEVEQLIKTWHIPVAGIIVEPIQAEGGDNYATPYFFQGLRDITKKHGVSMIVDEVQTGMGATGKFWAHEHWNLTSPPDIVTFSKKMQAAGFYHNLDYRPSESYRNFNTWMGDPVRALELEVVIGEIKKNHLLDNVVITGNYLKDGLFDIAARYPGLIQNIRGEGTFLAIDFPTPAERDRVISHIRLLGVEMGGCGERSIRFRPMLVCQPSHINQFLNRFDQTMKELYKN

SEQ ID NO: 71 - protein, 449 aa, Mycobacterium tuberculosis
MASLQQSRRLVTEIPGPASQALTHRRAAAVSSGVGVTLPVFVARAGGGIVEDVDGNRLIDLGSGIAVTTIGNSSPRVVDAVRTQVAEFTHTCFMVTPYEGYVAVAEQLNRITPGSGPKRSVLFNSGAEAVENAVKIARSYTGKPAVVAFDHAYHGRTNLTMALTAKSMPYKSGFGPFAPEIYRAPLSYPYRDGLLDKQLATNGELAAARAIGVIDKQVGANNLAALVIEPIQGEGGFIVPAEGFLPALLDWCRKNHVVFIADEVQTGFARTGAMFACEHEGPDGLEPDLICTAKGIADGLPLSAVTGRAEIMNAPHVGGLGGTFGGNPVACAAALATIATIESDGLIERARQIERLVTDRLTTLQAVDDRIGDVRGRGAMIAVELVKSGTTEPDAGLTERLATAAHAAGVIILTCGMFGNIIRLLPPLTIGDELLSEGLDIVCAILADL

SEQ ID NO: 72 - protein, 446 aa, Mycobacterium leprae
MTSVEQSRQLVTEIPGPVSLELAKRLNAAVPRGVGVTLPVFVTRAAGGVIEDVDGNRLIDLGSGIAVTTIGNSSPRVVDAVRAQVADFTHTCFIITPYEEYVAVTEQLNRITPGSGEKRSVLFNSGAEAVENSIKVARSYTRKPAVVAFDHAYHGRTNLTMALTAKSMPYKSGFGPFAPEIYRAPLSYPYRDGLLNKDLATDGKLAGARAINVIEKQVGADDLAAVIIEPIQGEGGFIVPAEGFLATLLDWCRKNNVMFIADEVQTGFARTGAMFACEHDGIVPDLICTAKGIADGLPLAAVTGRAEIMNAPHVSGLGGTFGGNPVACAAALATITTIENDGLIQRAQQIERLITDRLLRLQDADDRIGDVRGRGAMIAVELVKSGTAEPDPELTEKVATAAHATGVIILTCGMFGNIIRLLPPLTISDELLAEGLDILSRILGDF

SEQ ID NO: 73 - protein, 449 aa, Mycobacterium bovis; identical to SEQ ID NO: 71

SEQ ID NO: 74 - protein, 449 aa, Mycobacterium tuberculosis; identical to SEQ ID NO: 71

SEQ ID NO: 75 - protein, 470 aa, Streptomyces aizunensis
MRPMTAKIFAVDSVRPIDEFEQDALRVADVIRERGVCLGDRVMLKAGNSASYVCVLYALMHIGASIVLVDQQEHKEETRRIALRTGVKVTFVDDETPIDQDADPIHLYELMVATQNRPPMDSALSFDAWGELSDGLIMWTSGSTGSPKGVVKSGGKFLANLRRNAHQVGHRPDDVLMPLLPFAHQYGLSMVLIAWLTRCSLVIAPYRRLDRALRMARDSGTTVIDATPSSYRSILGLVTRKPALRAHLAGTRMFCVGAAPLDAPLVESYVQEFGLPLLDSYGSTELNNIAFATLDNPVSCGRAMEGIGLRIVDEDGREVAAGQPGEIEVDTPDALEGQIAEDGSIIPAPTGWQRTGDLGHLDADGNLYVLGRKFAVHRMGYTLYPELIERKVAAEGCPTRIVPLPDELRGSQLVFFVEDDEQRDAGYWRERLCGLLPAFEQPNKVVVLEQFPLNRNGKPDKKELTRMAAE

SEQ ID NO: 76 - protein, 238 aa, Artificial Sequence
  GFP protein
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 77 - protein, 236 aa, Artificial Sequence
  RFP protein
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 78 - DNA, 717 bp, Artificial Sequence
  GFP gene
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactggagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtccacacaatctgccctttcgaaagatcccaacgaaaagcgtgaccacatggtccttcttgagtttgtaactgctgctgggattacacatggcatggatgagctctacaaataa

SEQ ID NO: 79 - DNA, 711 bp, Artificial Sequence
  mCherry gene
atggtgtcaaagggagaggaagacaatatggcgattatcaaagagttcatgcgatttaaggtacacatggagggaagcgtgaatggccatgaatttgaaatagagggggaaggggagggacgcccatacgagggcacacaaacggctaaattgaaagttactaaaggaggcccgcttccttttgcgtgggatatactttcaccccaattcatgtacggttctaaagcatacgtcaagcacccagcagatatacctgattacttaaagctgagctttcctgaaggctttaaatgggaacgcgtaatgaactttgaagacggaggagtcgtcaccgtaacgcaggattcttccttacaggatggtgagtttatttataaggtaaaattacgtggcacaaattttccttctgacgggcctgtcatgcagaaaaaaaccatgggatgggaagcgtcttcagaaagaatgtatcctgaagacggggcactgaaaggagagataaagcagcgtttgaaactgaaagatggtggccactacgatgcggaagtaaaaacgacgtataaggcaaaaaaacccgtgcagctacctggtgcttataatgtgaatattaaacttgacataacgtcgcacaacgaggattacacaatcgtggaacagtacgaaagggccgagggccgccattcaacgggcggcatggatgaactgtataagtaa

SEQ ID NO: 80 - DNA, 1401 bp, Escherichia coli
atggataagaagcaagtaacggatttaaggtcggaactactcgattcacgttttggtgcgaagtctatttccactatcgcagaatcaaaacgttttccgctgcacgaaatgcgcgacgatgtcgcattccagattatcaatgacgaattatatcttgatggcaacgctcgtcagaacctggccactttctgccagacctgggacgacgaaaatgtccacaaattgatggatttatccattaacaaaaactggatcgacaaagaagaatatccgcaatccgcagccatcgacctgcgttgcgtaaatatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtacaaatctgctggcataaattcgcccgctactgggatgtggagctgcgtgagatccctatgcgccccggtcagttgtttatggacccgaaacgcatgattgaagcctgtgacgaaaacaccatcggcgtggtgccgactttcggcgtgacctacactggtaactatgagttcccacaaccgctgcacgatgcgctggataaattccaggccgataccggtatcgacatcgacatgcacatcgacgctgccagcggtggcttcctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctgggtggtcaaattggtacttttgccatcaacttctcccgcccggcgggtcaggtaattgcacagtactatgaattcctgcgcctcggtcgtgaaggctataccaaagtacagaacgcctcttaccaggttgccgcttatctggcggatgaaatcgccaaactggggccgtatgagttcatctgtacgggtcgcccggacgaaggcatcccggcggtttgcttcaaactgaaagatggtgaagatccgggatacaccctgtatgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccaacagaacagctttaaacatacctga

SEQ ID NO: 81 - DNA, 1377 bp, Sulfobacillus thermosulfidooxidans
atgaaaaaagcatcggaggaagtggcgattaatcccttgtttgcccgccgcggagaagaatcggttccccgatatcgtctctcggaagaaggattattgcctgaaacagcctatcaaattgttcatgatgaattgactttagacggcagtgctcggttaaacttggcgacttttgtgggaacgtggatggagccagaagctcagaaactgtatgtcgaagcggccaataaaaatatcatcgataaagatgaatatcctgagacggctgctattgaggaacggtgtctgaaaatcctggcaaacttatggaatgctccggatcccgccaatagtattggggtgtccacaacaggttcatcggaagcttgcatgttaggaggcctggctttaaaaagacggtggcaattatcccggcgtcaacaaggaaaatccaccgaccatcccaatattgtctttagttcagctgtccaagtcgtctgggaaaagtttgcgaattattttgaagtcgaaccccgctatgtgaacatcacgcccgaacatccttatttgacgccagaaggtgtgatcaaggcggtggacgaaaatactattggagttgtccccattttaggggtgacctataccggcagttacgaacccgttgaagctatcgcacaggctttagatgatttagaaaaacgcacgggcctgtccattcccattcatgtggatgcggcatcgggcggttttgtggcgcccttcttgcaaccagatttggtatgggattttagactgccgcgggtccattctattaatacctcaggccataaatacggactggtctatccggggctgggatgggttttatggcgggataagacgttattgccagaggaattaatcttcaatgtctcatatttagggggcagtatgccaacgtttggactcaatttttccagacccggggcccaagttctccttcaatactataattttcttcggttgggacgtcagggctattacacggtccaaaagacgtgtcaaaatgttgctcattttttggcacaggaaattggggctatggagccgtttgagctggtaactgacggatcggatttgccggtatttgcctggcgaaaaagaccggaagagaatcctttatggtctttagaggatttatcttttgtattgcgggaacggggatggcaagtaccagcctatccgttaccaccaactatgcaagacgttaccataatgcgcattgttgttcgcaacagtttttctatggatatggcacagctctttttggatgacttaaaccgggcggtggcgtatttacaggggttagatcatccctttccccaggctttacgccaacataaaccgtttgctcattga

SEQ ID NO: 82 - DNA, 1407 bp, Lactobacillus brevis
atggctatgttgtatggaaaacacacgcatgaaacagatgagacgctcacgccaatcttcggggccacagctgaacgccacgacctccccaaatataaattggcaaagcacgcgctcgagccccgtgaagccgatcgattggttcgcgatcaactattggatgaaggaaactcgcggctgaatctcgccacgttctgtcagacttacatggaaccggaagcggttgaactcatgaaagatacactggagaaaaacgccatcgataaatccgagtatcctcggaccgctgaaattgaaaatcgttgcgttaatatcattgccaacctctggcatgctccagaagctgagtcgttcactggcacctcgacgattggttcctccgaggcctgcatgctggccggtttggcgatgaagtttgcttggcgtaagcgcgccaaagcgaacggtcttgacttaactgcccatcaacctaatattgtcatctcagccggttatcaagtttgttgggaaaaattctgtgtctattgggacatcgacatgcatgtcgttcccatggacgatgaccacatgtccttgaatgtcgatcacgtgttagattacgtggatgactacaccattggtatcgttggcattatgggcatcacttatactggacaatacgacgatttagcccgattagatgccgttgtagagcggtacaatcggacgactaagttcccggtatatatccatgtcgatgccgcttccggcggattttacacgccgtttattgaacccgagctcaagtgggacttccgtttaaacaacgtgatttccatcaatgcctccggccacaaatatggcttggtttatcccggagtcggctgggtaatctggcgtgaccaacagtatctaccaaaagagctggtctttaaggtcagctacttgggtggtgaactacctacgatggccatcaacttctcccacagtgcctcccaattaatcggtcagtattacaactttattcgctttggttttgatggctatcgtgaaattcaagaaaaaactcacgacgttgcccgctatctcgcgaaatcgctcactaaattagggggcttttccctcattaatgacggccacgagttaccgctgatctgttatgaactcactgccgattctgatcgcgaatggaccctctacgatttatccgatcggttattaatgaagggctggcaggttcccacctatcccttaccaaaaaacatgacggaccgcgttattcaacggatcgtggttcgggctgactttggtatgagtatggcccacgactttattgatgatctaacccaagccattcacgatctcgaccaagcacacatcgttttccatagtgatccgcaacctaaaaaatacgggttcacgcactaa

SEQ ID NO: 83 - DNA, 1401 bp, Artificial Sequence
  E. coli GAD mutant E89Q gene
atggataagaagcaagtaacggatttaaggtcggaactactcgattcacgttttggtgcgaagtctatttccactatcgcagaatcaaaacgttttccgctgcacgaaatgcgcgacgatgtcgcattccagattatcaatgacgaattatatcttgatggcaacgctcgtcagaacctggccactttctgccagacctgggacgacgaaaatgtccacaaattgatggatttatccattaacaaaaactggatcgacaaagaacagtatccgcaatccgcagccatcgacctgcgttgcgtaaatatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtacaaatctgctggcataaattcgcccgctactgggatgtggagctgcgtgagatccctatgcgccccggtcagttgtttatggacccgaaacgcatgattgaagcctgtgacgaaaacaccatcggcgtggtgccgactttcggcgtgacctacactggtaactatgagttcccacaaccgctgcacgatgcgctggataaattccaggccgataccggtatcgacatcgacatgcacatcgacgctgccagcggtggcttcctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctgggtggtcaaattggtacttttgccatcaacttctcccgcccggcgggtcaggtaattgcacagtactatgaattcctgcgcctcggtcgtgaaggctataccaaagtacagaacgcctcttaccaggttgccgcttatctggcggatgaaatcgccaaactggggccgtatgagttcatctgtacgggtcgcccggacgaaggcatcccggcggtttgcttcaaactgaaagatggtgaagatccgggatacaccctgtatgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccaacagaacagctttaaacatacctga

SEQ ID NO: 84 - DNA, 1335 bp, Artificial Sequence
  Sth GAD mutant E84Q delta C14 gene
atgaaaaaagcatcggaggaagtggcgattaatcccttgtttgcccgccgcggagaagaatcggttccccgatatcgtctctcggaagaaggattattgcctgaaacagcctatcaaattgttcatgatgaattgactttagacggcagtgctcggttaaacttggcgacttttgtgggaacgtggatggagccagaagctcagaaactgtatgtcgaagcggccaataaaaatatcatcgataaagatcaatatcctgagacggctgctattgaggaacggtgtctgaaaatcctggcaaacttatggaatgctccggatcccgccaatagtattggggtgtccacaacaggttcatcggaagcttgcatgttaggaggcctggctttaaaaagacggtggcaattatcccggcgtcaacaaggaaaatccaccgaccatcccaatattgtctttagttcagctgtccaagtcgtctgggaaaagtttgcgaattattttgaagtcgaaccccgctatgtgaacatcacgcccgaacatccttatttgacgccagaaggtgtgatcaaggcggtggacgaaaatactattggagttgtccccattttaggggtgacctataccggcagttacgaacccgttgaagctatcgcacaggctttagatgatttagaaaaacgcacgggcctgtccattcccattcatgtggatgcggcatcgggcggttttgtggcgcccttcttgcaaccagatttggtatgggattttagactgccgcgggtccattctattaatacctcaggccataaatacggactggtctatccggggctgggatgggttttatggcgggataagacgttattgccagaggaattaatcttcaatgtctcatatttagggggcagtatgccaacgtttggactcaatttttccagacccggggcccaagttctccttcaatactataattttcttcggttgggacgtcagggctattacacggtccaaaagacgtgtcaaaatgttgctcattttttggcacaggaaattggggctatggagccgtttgagctggtaactgacggatcggatttgccggtatttgcctggcgaaaaagaccggaagagaatcctttatggtctttagaggatttatcttttgtattgcgggaacggggatggcaagtaccagcctatccgttaccaccaactatgcaagacgttaccataatgcgcattgttgttcgcaacagtttttctatggatatggcacagctctttttggatgacttaaaccgggcggtggcgtatttacaggggttagatcattga

SEQ ID NO: 85 - DNA, 1407 bp, Artificial Sequence
  Lbr GAD 4AA mutant gene
atggccatgctgtatggtaagcacacccatgaaactgatgagaccctcatacccatattcggtgccactgccgaacgtcacgacctcccaaagtataagctggcgaagcacgcgctcgagccacgtgaagccgatcgtctggtgcgtgatcagttgctggatgaaggtaactcccgcttgaatctcgccaccttctgccaaacttacatggaaccagaagcggtggaactcatgaaggatactttggagaagaacgccatagataagtccgagtatccacgcaccgccgaaatcgaaaatcgttgtgtgaatataatcgccaacctctggcatgcccccgaagccgagtccttcactggtacctccaccatcggctcctccgaggcctgtatgttggccggcctggcgatgaagtttgcctggcgtaagcgtgccaaggcgaacggcctcgacctgactgcccatcagccaaatatcgtcataagcgccggctatcaggtgtgctgggaaaagttctgcgtctattgggacatagacatgcatgtcgtgccaatggacgatgaccacatgtccctgaatgtcgatcacgtgctggattacgtggatgactacaccatcggcatagtgggtatcatgggtataacttatactggtcagtacgacgatctggcccgtctggatgccgtggtagagcgctacaatcgcaccactaagttcccagtatatatacatgtcgatgccgcctccggtggtttttacaccccatttatcgaaccagagctcaagtgggacttccgtctgaacaacgtgatctccataaatgcctccggtcacaagtatggtctggtgtatccaggtgtcggttgggtaatatggcgtggtcagcaatatttgcccaaggagttggtctttaaggtctcctacctgggcggcagcttgccaaccatggccataaacttctcccacagcgcctcccagctgataggccaatattacaactttatccgttttggctttgatggttatcgtgaaatccatgaaaagactcacgacgtggcccgttatctcgcgaagtccctcactaagctgggtggtttttccctcatcaatgacggtcacgagctgccattgatatgctatgaactcactgccgattccgatcgtgaatggaccctctacgatctgtccgatcgcctgctgatgaagggttggcaagtgccaacctatccactgcccaagaacatgaccgaccgtgtgatccagcgcatagtggtgcgcgccgactttggcatgagcatggcccacgactttatcgatgatttgacccaggccatccacgatctcgaccaggcgcacatagtgttccatagcgatccacagccaaagaagtacggtttcacccactaa

SEQ ID NO: 86 - DNA, 1845 bp, Artificial Sequence
  Egr kdc (harmonized) gene
atgggcatcgccctggatggtcctgaccgcgaccgcatgttcaacgcctacttgcattcttcaaggccctcctttggaccggccaccgatgtgttcgtgcagtgcttggaggcagaaaagatcgactggatctttggaatccccggggaggagaacttggacctcctccacagcctggcaaaggcgcaccgccgggagggtggggagacgatgaagctcgtggtgacccggcatgagcaggcagctgggtttatggcagcgactgtgggccggctgacgggacaccctgcggcttgcctgtcaaccctcgggcctggagccaccaacttcaccaccgctctggcgtacagcaagttgggcggtttccctttgatctgtatcactgggcagaagcccatccgtcacagcaaacagggtgcctttcagatcctggacatctgcggtcacttccgggatgtggtgaagtccacaaagagcatcgaggaccccgacctcatccccagcacaattcgcaatgccgtgctgcaggcgcgagaggagaagcctggccccgtccacattgaactggccgaggacattgccgccctggaggtctccaaggagggcggctgggtgttccccgcccccacccggacgcccatcgcccgcggccgtcgtccgctggtggagcccaagtgcattgagcacgccgtgacccttattcatgccgccaagcgcccgctgatttgtatcgccgctggagccaaccgcaagaacacccaccatgctatggaggtgttgatgcggaagacggggctgatggtggtgtgcacacagatgggcaagggggttgtgtacgaggacaacctcggatacatcggctgcacggccctgtcggacaaggacctggtgcacgtggccatcaacaccgcggacctcatcgtcaacgtgggccacgacatcagcgagaagccgccgttcatcatgaaccacaacaagccccccctggtcatccacgccaacttcacccggcccctggtggaccccgtgtacttcccacacctcgtgctggttggagacatcaacgactgcctctggcagctcgcgacccgcatccggcctcagccacactgggacttcaaccccttccagatggtccgccgggagatcgagcgcaccgtgtggaacagcccctacgcccagtccgatgcgttcccactgacggtccagcgcctcgtgtcggacgtccggaaggcgatgccggtggatggtatcgtcagcctcgacaatggcatgtacaagatctggtttgcccggcagtacaagaccatgatgggcaacaccctgctgttggacaatgcgttggccaccatgggtgctggactgccctcttgcattgcggccaagctagtgtaccctgagcgggtctgcatcgcagtgtgcggcgatggcgggttcatgatgaacagccaggagatcgagacagcattgaggctgaaccttcacattgtggtcatcgtgctcaacaacaatagctatggcatgattgcctggaaggcgactgccatgggcatggacgacttcgggctgaactacggcaacccggacttcgcccagtacgcccgcgcctacggggccatcggccacaacgtcaagtccacagccgagttcctccccaccctggagaaggccatcaaagagcacggcgtgcacatcatcgacctccccatctcgtacgagacgtcggacaaggccctttttgaggacctgcccaaggaagtggaggagctcaagaaggcggtggccaaagccatctccgaggagaagaagttcgactgggatgccgtgacggcagctcagagctag

SEQ ID NO: 87 - DNA, 1638 bp, Artificial Sequence
  Ssc kdc (harmonized) gene
atgaacactgctgaattgttgattcggtgcctcgaaaacgaaggcgtggagtatatcttcggcttgccaggcgaagaaaacctgcatattctggaagccctcaaagagtccccgattcgcttcattaccgtccgccatgaacaaggcgccgctttcatggccgacgtgtatggccgtttgaccggcaaggctggcgtctgcttgtccaccttgggcccaggcgctaccaacctcatgactggcgtcgccgacgcaaatctggacggcgcaccgttgatcgcaatcactggccaagtgggcaccgatcgcatgcatatcgaaagccatcagtatctcgacttggtggcaatgttcgccccggtcaccaaatggaacaagcagatcgtccggccaaatacgaccccagaggtggtacgtcgtgccttcaagatcgcccaacaagaaaagccaggcgctgtacatattgacctgccagaaaacatcgcagcaatgccggtagaaggccaaccgctgcaacgcgacggccgtgaaaagatttatgctagtagccgcagcttgaatcgcgctgccgaggcaattgcccatgccaaaagcccattgatcttggtgggcaacggcatcatccgcgccgacgccgccgaagccctgaccgacttcgccacccaattgaacatcccggtagtcaataccttcatgggcaagggcgctatcccgtacacccatccgttgagcttgtggacggtaggtctgcagcaacgcgacttcgtcacctgcgccttcgaacaaagcgacttggtgatcgctgtgggctacgacttgattgaatatagcccgaagcgctggaatccagagggtacgaccccaatcattcatatcggcgaagtggccgccgaaatcgacagcagctatatcccgctgactgaagtcgtcggcgatatcggcgacgccttgaacgaaatccgtaagcgcactgatcgtgagggcaagaccgcaccaaagttcctgaacgtccgcgctgagatccgcgaggattatgaacgccatggcaccgatgctagcttcccagtcaagccgcagaagattatttacgacctgcgccaggtgatggccccagaggatattgtcatctccgacgtgggcgcccataagatgtggatggcccgccattaccattgtgaccgcccgaacacttgtttgatcagcaacggtttcgcagcaatgggcatcgcaatcccgggcgctgtagctgccaagttggtctacccagaaaagaacgtcgtggctgtcactggcgatggcggtttcatgatgaattgtcaagagctggaaacggccttgcgcatcggcgcaaatttcgtcaccctcatctttaacgacggcggctatggcttgattggctggaagcaaatcaatcaatttggcgctccagccttcgtggagttcggcaacccggacttcgtgcaattcgccgaaagcatgggcctgaaaggctatcgcatcaccgccgccgccgatctcgtgccaaccttgaaggaagccctcgcccaagacgtaccagcagtgattgactgtccggtggattacagcgagaacgtgaagtttagccagaagagtggcgacttgatttgtcgtatgtga

SEQ ID NO: 88 - DNA, 1281 bp, Escherichia coli
atgaacagcaataaagagttaatgcagcgccgcagtcaggcgattccccgtggcgttgggcaaattcacccgattttcgctgaccgcgcggaaaactgccgggtgtgggacgttgaaggccgtgagtatcttgatttcgcgggcgggattgcggtgctcaataccgggcacctgcatccgaaggtggtggccgcggtggaagcgcagttgaaaaaactgtcgcacacctgcttccaggtgctggcttacgagccgtatctggagctgtgcgagattatgaatcagaaggtgccgggcgatttcgccaagaaaacgctgctggttacgaccggttccgaagcggtggaaaacgcggtaaaaatcgcccgcgccgccaccaaacgtagcggcaccatcgcttttagcggcgcgtatcacgggcgcacgcattacacgctggcgctgaccggcaaggtgaatccgtactctgcgggcatggggctgatgccgggtcatgtttatcgcgcgctttatccttgcccgctgcacggcataagcgaggatgacgctatcgccagcatccaccggatcttcaaaaatgatgccgcgccggaagatatcgccgccatcgtgattgagccggttcagggcgaaggcggtttctacgcctcgtcgccagcctttatgcagcgtttacgcgctctgtgtgacgagcacgggatcatgctgattgccgatgaagtgcagagcggcgcggggcgtaccggcacgctgtttgcgatggagcagatgggcgttgcgccggatcttaccacctttgcgaaatcgatcgcgggcggcttcccgctggcgggcgtcaccgggcgcgcggaagtaatggatgccgtcgctccaggcggtctgggcggcacctatgcgggtaacccgattgcctgcgtggctgcgctggaagtgttgaaggtgtttgagcaggaaaatctgctgcaaaaagccaacgatctggggcagaagttgaaagacggattgctggcgatagccgaaaaacacccggagatcggcgacgtacgcgggctgggggcgatgatcgccattgagctgtttgaagacggcgatcacaacaagccggacgccaaactcaccgccgagatcgtggctcgcgcccgcgataaaggcctgattcttctctcctgcggcccgtattacaacgtgctgcgcatccttgtaccgctcaccattgaagacgctcagatccgtcagggtctggagatcatcagccagtgttttgatgaggcgaagcagtag

SEQ ID NO: 89 - DNA, 1413 bp, Streptomyces aizunensis
atgcgccccatgaccgccaaaattttcgcagtcgattcggtccgccctatcgatgagtttgagcaagatgcattacgtgtagcagacgtcattcgtgaacgtggtgtatgccttggtgatcgtgtcatgttaaaagcaggtaattcggcctcgtatgtctgcgtcttatatgcacttatgcacattggagctagtattgttttagttgatcaacaagaacacaaagaggagactcgtcgtattgcccttcgtacgggtgttaaagtcacgtttgtcgatgatgagacgcctattgatcaagatgcagatcccattcacctttatgaattaatggtcgcgacgcaaaatcgcccaccaatggatagtgcgttgtcttttgatgcatggggtgagctcagtgatggactcattatgtggacgagcggttccacgggtagcccaaaaggtgttgttaaatccggtggcaaatttctcgctaatttgcgtcgtaatgcacaccaagtaggacaccgtcctgatgatgttcttatgccccttttaccatttgcacaccaatatgggttgtcgatggtcttgattgcgtggcttacacgttgttcccttgttattgcgccctatcgtcgtttagatcgtgcccttcgtatggcgcgtgattcgggtacaaccgttattgatgccactccttcgtcatatcgtagcattttgggtttagtcacacgcaaacccgcattgcgtgcccacctcgctggtacacgtatgttttgcgttggagccgcgcccttggatgccccacttgttgagtcgtatgtgcaagagtttggtttgccattactcgatagctatggttcaacagagctcaataatattgcgtttgcaactcttgataatcccgtaagttgtgggcgtgcgatggaaggtattggcttacgtattgttgatgaggatgggcgtgaggttgccgctggccaacccggtgagattgaggtagatacgcccgatgcgttggaagggcaaatcgcagaggatggctcgattattcccgcccctacgggttggcaacgtaccggggatctcggtcacttggatgccgatggaaatttatatgtactcggacgtaaatttgcggtacaccgtatggggtatacattataccctgagttgattgagcgtaaagtcgcggctgaggggtgtcctacgcgtattgtacctttacccgatgaattgcgtggttcgcaacttgttttctttgtcgaggatgatgagcaacgtgatgcggggtattggcgtgagcgtctttgtggattattgccagcatttgagcaacctaataaagtagttgtcttggagcaatttccccttaatcgtaatggaaaacccgataagaaagagttaactcgtatggcggcagaataa

SEQ ID NO: 90 - protein, 452 aa, Artificial Sequence
  E. coli GAD mutant E89Q delta C14
MDKKQVTDLRSELLDSRFGAKSISTIAESKRFPLHEMRDDVAFQIINDELYLDGNARQNLATFCQTWDDENVHKLMDLSINKNWIDKEQYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHP

SEQ ID NO: 91 - DNA, 1359 bp, Artificial Sequence
  E. coli GAD mutant E89Q delta C14 gene
atggataagaagcaagtaacggatttaaggtcggaactactcgattcacgttttggtgcgaagtctatttccactatcgcagaatcaaaacgttttccgctgcacgaaatgcgcgacgatgtcgcattccagattatcaatgacgaattatatcttgatggcaacgctcgtcagaacctggccactttctgccagacctgggacgacgaaaatgtccacaaattgatggatttatccattaacaaaaactggatcgacaaagaacagtatccgcaatccgcagccatcgacctgcgttgcgtaaatatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtacaaatctgctggcataaattcgcccgctactgggatgtggagctgcgtgagatccctatgcgccccggtcagttgtttatggacccgaaacgcatgattgaagcctgtgacgaaaacaccatcggcgtggtgccgactttcggcgtgacctacactggtaactatgagttcccacaaccgctgcacgatgcgctggataaattccaggccgataccggtatcgacatcgacatgcacatcgacgctgccagcggtggcttcctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctgggtggtcaaattggtacttttgccatcaacttctcccgcccggcgggtcaggtaattgcacagtactatgaattcctgcgcctcggtcgtgaaggctataccaaagtacagaacgcctcttaccaggttgccgcttatctggcggatgaaatcgccaaactggggccgtatgagttcatctgtacgggtcgcccggacgaaggcatcccggcggtttgcttcaaactgaaagatggtgaagatccgggatacaccctgtatgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgtga

SEQ ID NO: 92 - protein, 443 aa, Artificial Sequence
  S. thermosulfidooxidans GAD mutant E84Q delta C14
MKKASEEVAINPLFARRGEESVPRYRLSEEGLLPETAYQIVHDELTLDGSARLNLATFVGTWMEPEAQKLYVEAANKNIIDKDQYPETAAIEERCLKILANLWNAPDPANSIGVSTTGSSEACMLGGLALKRRWQLSRRQQGKSTDHPNIVFSSAVQVVWEKFANYFEVEPRYVNITPEHPYLTPEGVIKAVDENTIGVVPILGVTYTGSYEPVEAIAQALDDLEKRTGLSIPIVDAASGGFVAPFLQPDLVWDFRLPRVHSINTSGHKYGLVYPGLGWVLWRDKTLLPEELIFNVSYLGGSMPTFGLNFSRPGAQVLLQYYNFLRLGRQGYYTVQKTCQNVAHFLAQEIGAMEPFELVTDGSDLPVFAWRKRPEENPLWSLEDLSFVLRERGWQVPAYPLPPTMQDVTIMRIVVRNSFSMDMAQLFLDDLNRAVAYLQGLDH